(19) United States
(12) Patent Application Publication
(10) Pub. No.: US 2024/0276875 A1
(43) Pub. Date: Aug. 15, 2024

(54) COMPOSITION, LIGHT-EMITTING DEVICE INCLUDING THE COMPOSITION, AND ELECTRONIC APPARATUS INCLUDING THE LIGHT-EMITTING DEVICE

(71) Applicant: Samsung Electronics Co., Ltd., Suwon-si (KR)

(72) Inventors: Jong Soo KIM, Suwon-si (KR); Giwook KANG, Yongin-si (KR); Eunsuk KWON, Suwon-si (KR); Eunhye AN, Yongin-si (KR); Dongjin JANG, Yongin-si (KR); Yeon Sook CHUNG, Suwon-si (KR); Yongsik JUNG, Suwon-si (KR); Eunjeong CHOI, Yongin-si (KR)

(21) Appl. No.: 18/413,103

(22) Filed: Jan. 16, 2024

(30) Foreign Application Priority Data
Jan. 17, 2023 (KR) 10-2023-0006987

(51) Int. Cl. H10K 85/60; C07D 209/86; C07D 403/14; C07F 7/08; C09K 11/06; H10K 50/12; H10K 85/40; H10K 101/00

(52) U.S. Cl. CPC H10K 85/6572; C07D 209/86; C07D 403/14; C07F 7/0812; C09K 11/06; H10K 85/40; H10K 85/654; C09K 2211/1018; H10K 50/121; H10K 2101/90

(57) ABSTRACT

A composition includes a first compound represented by Formula 1 and a second compound including an azine group including at least one nitrogen, a light-emitting device including the composition, and an electronic apparatus including the light-emitting device:

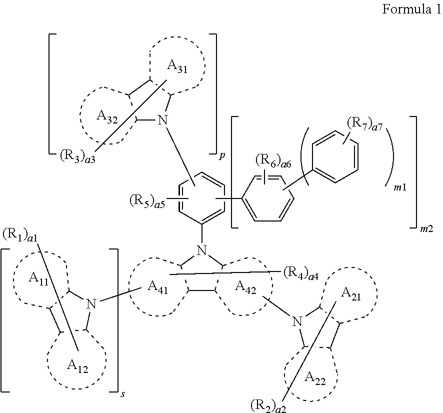

wherein the detailed description of Formula 1 is provided herein.

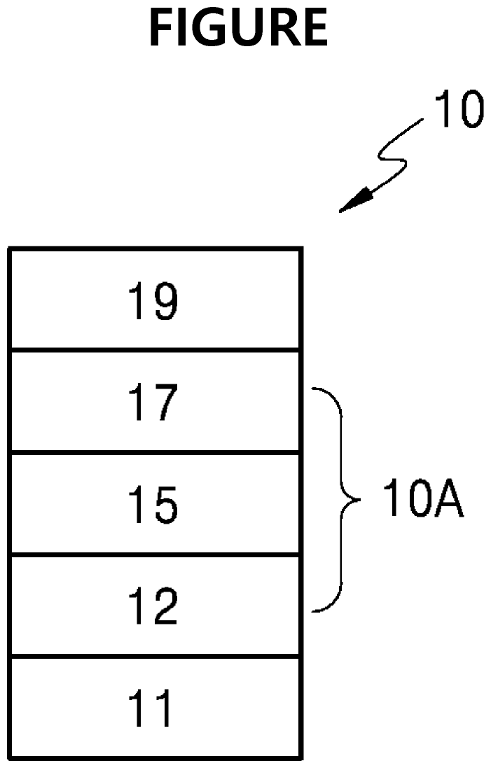

### CROSS-REFERENCE TO RELATED APPLICATION

This application claims priority under 35 U.S.C. § 119 to Korean Patent Application No. 10-2023-0006987, filed on Jan. 17, 2023, in the Korean Intellectual Property Office, the content of which is herein incorporated by reference in its entirety.

### BACKGROUND

### 1. Field

The disclosure relates to compositions, light-emitting devices including the compositions, and electronic apparatuses including the light-emitting devices.

### 2. Description of the Related Art

From among light-emitting devices, organic light-emitting devices are self-emissive devices that, as compared with devices in the art, have wide viewing angles, high contrast ratios, short response times, and excellent characteristics in terms of luminance, driving voltage, and response speed, and produce full-color images.

In an example, an organic light-emitting device includes an anode, a cathode, and an interlayer that is arranged between the anode and the cathode and includes an emission layer. A hole transport region may be between the anode and the emission layer, and an electron transport region may be between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. The excitons may transition from an excited state to a ground state, thus generating light.

The need remains for novel materials for organic light-emitting devices.

### SUMMARY

Provided are a composition including a certain compound and a layer including the compound. A light-emitting device including the composition has excellent lifespan characteristics, and thus, a high-quality electronic apparatus using the light-emitting device may be implemented.

Additional aspects will be set forth in part in the description which follows and, in part, will be apparent from the description, or may be learned by practice of the presented embodiments of the disclosure.

According to an aspect of the disclosure, a composition includes
a first compound represented by Formula 1, and
a second compound including an azine group including at least one nitrogen.

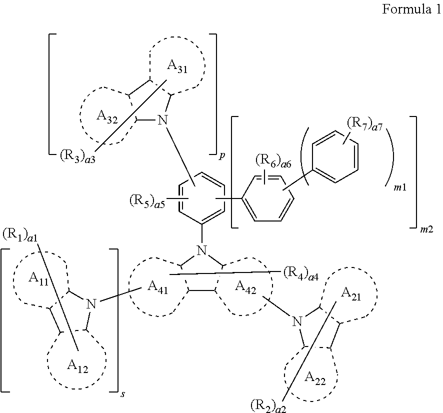

### In Formula 1,

rings A₁₁, A₁₂, A₂₁, A₂₂, A₃₁, A₃₂, A₄₁, and A₄₂ are each a pi (π) electron-rich C₅-C₆₀ cyclic group,
R₁ to R₃ are each independently hydrogen, deuterium, a C₁-C₆₀ alkyl group, or a deuterated C₁-C₆₀ alkyl group; a π electron-rich C₅-C₆₀ cyclic group unsubstituted or substituted with deuterium, a C₁-C₆₀ alkyl group, a π electron-rich C₅-C₆₀ cyclic group, or a combination thereof; or
—Si(B′₁)(B′₂)(B′₃),
wherein B′₁, B′₂, and B′₃ are each a π electron-rich C₅-C₆₀ cyclic group unsubstituted or substituted with at least one W′₁,
R₄ to R₇ and W′₁ are each independently
hydrogen, deuterium, a C₁-C₆₀ alkyl group, or a deuterated C₁-C₆₀ alkyl group; or
a phenyl group, or a biphenyl group, each unsubstituted or substituted with deuterium, a C₁-C₆₀ alkyl group, a phenyl group, a biphenyl group, or a combination thereof,
a1 to a4 are each independently an integer from 0 to 20,
a5 is an integer from 0 to 4,
a6 and a7 are each independently an integer from 0 to 5,
p and s are each independently 0 or 1, wherein p+s is 1 or 2,
m1 is 0, 1, 2, or 3, and
m2 is 1 or 2.

The composition may further include an emitter.

The emitter may emit blue light.

The composition may further include a sensitizer.

According to another aspect of the disclosure, a layer includes the composition.

The layer may be an emission layer.

According to another aspect of the disclosure, a light-emitting device includes the composition.

The light-emitting device may include a first electrode, a second electrode, and an interlayer arranged between the first electrode and the second electrode and including an emission layer, wherein the interlayer may include the composition.

The composition may be included in the emission layer of the light-emitting device. The emission layer may emit blue light.

According to another aspect of the disclosure, an electronic apparatus includes the light-emitting device.

### BRIEF DESCRIPTION OF THE DRAWING

The above and other aspects, features, and advantages of certain embodiments of the disclosure will be more apparent from the following description taken in conjunction with the FIGURE which is a schematic view of an organic light-emitting device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly on the other element or intervening elements may be present therebetween. In contrast, when an element is referred to as being “directly on” another element, there are no intervening elements present.

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise.

“Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

In an embodiment, a composition may include:
a first compound represented by Formula 1; and
a second compound including an azine group (for example, a pyridine group, a pyrimidine group, a pyrazine group, a pyridazine group, or a triazine group) including at least one nitrogen:

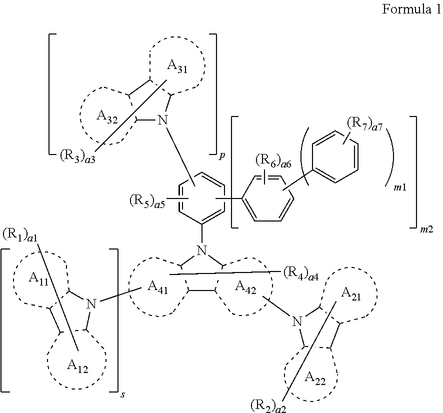

wherein, in Formula 1, rings A₁₁, A₁₂, A₂₁, A₂₂, A₃₁, A₃₂, A₄₁, and A₄₂ may each be a π electron-rich C₅-C₆₀ cyclic group.

The π electron-rich C₅-C₆₀ cyclic group may be, for example, a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, a furan group, a thiophene group, an isoindole group, an indole group, a benzofuran group, a benzothiophene group, a benzosilole group, a naphthopyrrole group, a naphthofuran group, a naphthothiophene group, a naphthosilole group, a benzocarbazole group, a dibenzocarbazole group, a dibenzofuran group, a dibenzothiophene group, a carbazole group, a dibenzosilole group, an indenocarbazole group, an indolocarbazole group, a benzofurocarbazole group, a benzothienocarbazole group, a benzosilolocarbazole group, a triindolobenzene group, a pyrrolophenanthrene group, a furanophenanthrene group, a thienophenanthrene group, a benzonaphthofuran group, a benzonapthothiophene group, an (indolo)phenanthrene group, a (benzofurano)phenanthrene group, or a (benzothieno)phenanthrene group.

For example, rings A₁₁, A₁₂, A₂₁, A₂₂, A₃₁, A₃₂, A₄₁, and A₄₂ in Formula 1 may each be a benzene group.

R₁ to R₃ in Formula 1 may each independently be:
hydrogen, deuterium, a C₁-C₆₀ alkyl group, or a deuterated C₁-C₆₀ alkyl group;
a π electron-rich C₅-C₆₀ cyclic group unsubstituted or substituted with deuterium, a C₁-C₆₀ alkyl group, a π electron-rich C₅-C₆₀ cyclic group, or a combination thereof; or
—Si(B′₁)(B′₂)(B′₃),
wherein B′₁, B′₂, and B′₃ may each be a π electron-rich C₅-C₆₀ cyclic group unsubstituted or substituted with at least one W′₁.

For example, B′₁, B′₂, and B′₃ may each be a benzene group unsubstituted or substituted with at least one W′₁.

In an embodiment, R₁ to R₃ in Formula 1 may each independently be:
hydrogen, deuterium, a C₁-C₂₀ alkyl group, or a deuterated C₁-C₂₀ alkyl group; or
a phenyl group or an N-carbazolyl group, each unsubstituted or substituted with deuterium, a C₁-C₂₀ alkyl group, a phenyl group, an N-carbazolyl group, or a combination thereof.

Herein, “N-carbazolyl group” refers to a monovalent group represented by

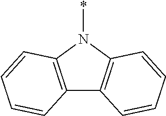 [SMILES: *n1c2ccccc2c2ccccc21]

(here, * indicates a binding site to a neighboring atom) that may be separated from N of a carbazole group and linked to another group.

R₄ to R₇ and W′₁ in Formula 1 may each independently be:
hydrogen, deuterium, a C₁-C₆₀ alkyl group, or a deuterated C₁-C₆₀ alkyl group; or
a phenyl group or a biphenyl group, each unsubstituted or substituted with deuterium, a C₁-C₆₀ alkyl group, a phenyl group, a biphenyl group, or a combination thereof.

For example, R₄ to R₇ and W′₁ in Formula 1 may each independently be:
hydrogen, deuterium, a C₁-C₂₀ alkyl group, or a deuterated C₁-C₂₀ alkyl group; or
a phenyl group unsubstituted or substituted with deuterium, a C₁-C₁₀ alkyl group, a phenyl group, or a combination thereof.

The a1 to a4 in Formula 1 indicate the numbers of R₁ to R₄, respectively, and may each independently be an integer from 0 to 20 (for example, 0 to 8).

The sum or a1 and a2 may be at least one and the at least one of R₁ and R₂ may be a tert-butyl group. The sum or a1 and a2 may be one and either R₁ or R₂ may be a tert-butyl group. The sum or a1 and a2 may be two and two R₁(s), two R₂(s), or one R₁ and one R₂ may be a tert-butyl group. The sum or a1 and a2 may be three and three R₁(s), or three R₂(s), or two R₁(s) and one R₂, or one R₁ and two R₂(s) may be a tert-butyl group. The sum or a1 and a2 may be four and two R₁(s) and two R₂(s) may be a tert-butyl group

The a5 in Formula 1 indicates the number of R₅ and may be an integer from 0 to 4.

The a6 and a7 in Formula 1 indicate the number of R₆ and R₇, respectively, and may each independently be an integer from 0 to 5.

The p and s in Formula 1 may each independently be 0 or 1, wherein p+s may be 1 or 2.

In an embodiment, p in Formula 1 may be 0, and s may be 1.

In one or more embodiments, p in Formula 1 may be 1, and s may be 0.

In one or more embodiments, p and s in Formula 1 may each be 1.

The m1 in Formula 1 may be 0, 1, 2, or 3.

For example, m1 may be 0, 1, or 2.

The m2 in Formula 1 may be 1 or 2.

In an embodiment, p in Formula 1 may be 0, and a group represented by

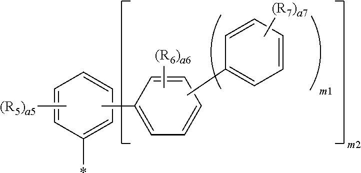

### in Formula 1 may be:

a group represented by one of Formulae ph-1 to ph-18; or
a group one of Formulae ph-1 to ph-18, in which at least one hydrogen is substituted with deuterium:

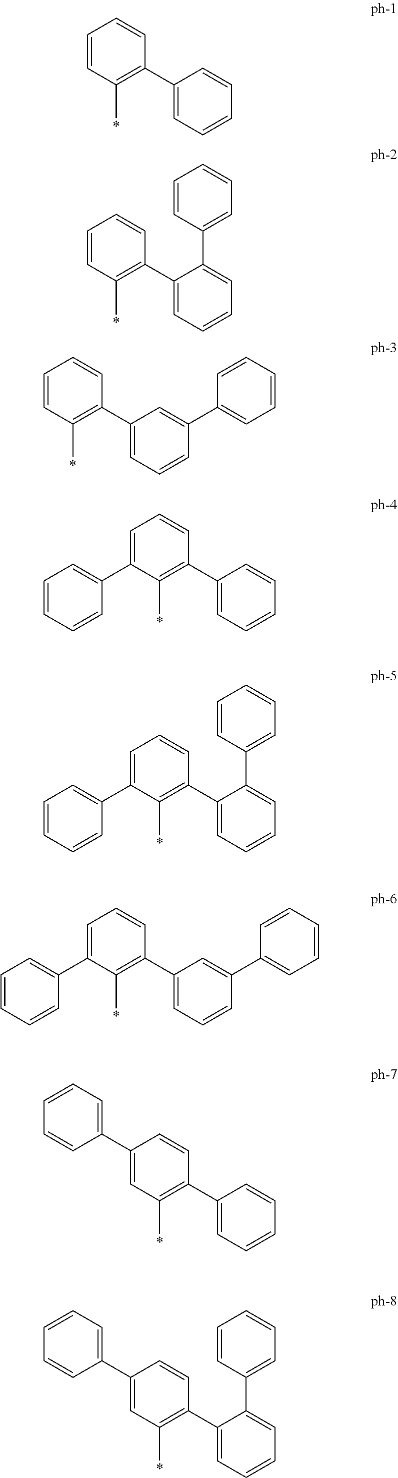

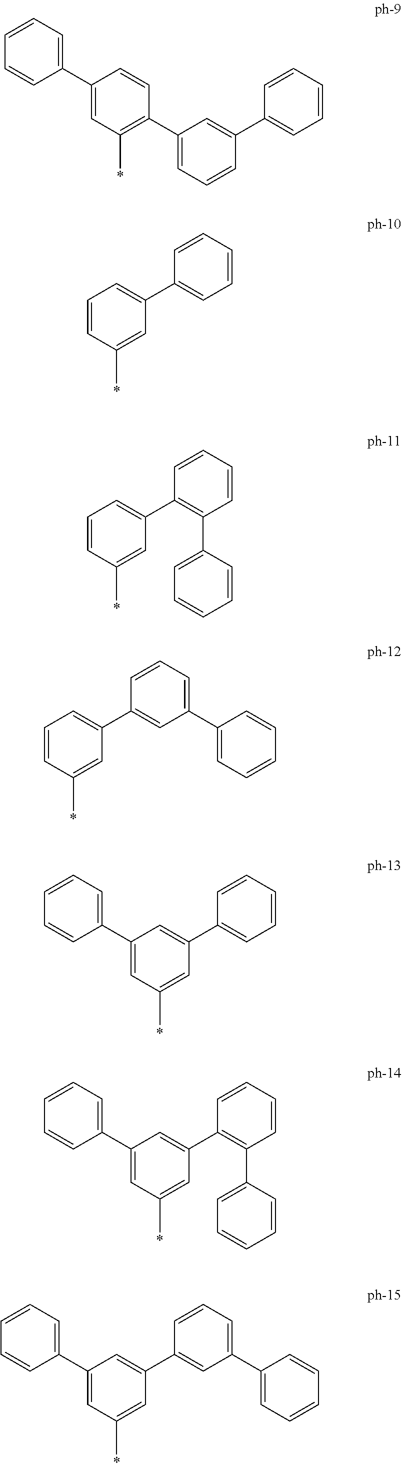

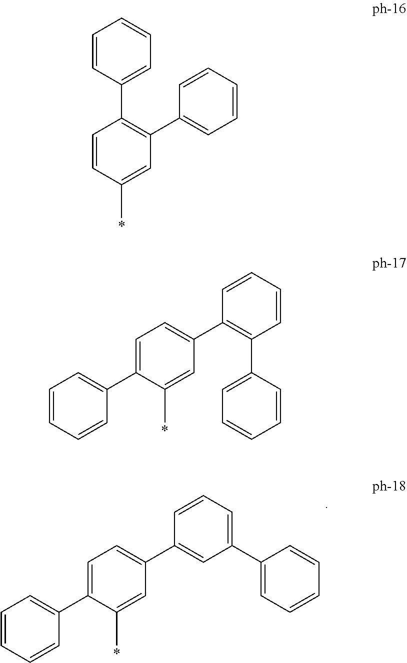

* in Formulae ph-1 to ph-18 indicates a binding site to a neighboring atom.

In one or more embodiments, p in Formula 1 may be 1, and a group represented by

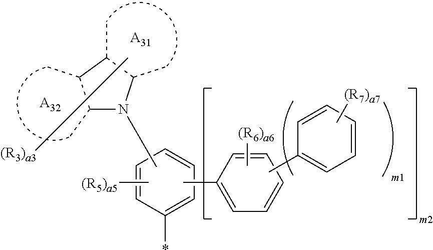

### in Formula 1 may be:

a group represented by one of Formulae ph-51 to ph-58; or
a group one of Formulae ph-51 to ph-58, in which at least one hydrogen is substituted with deuterium:

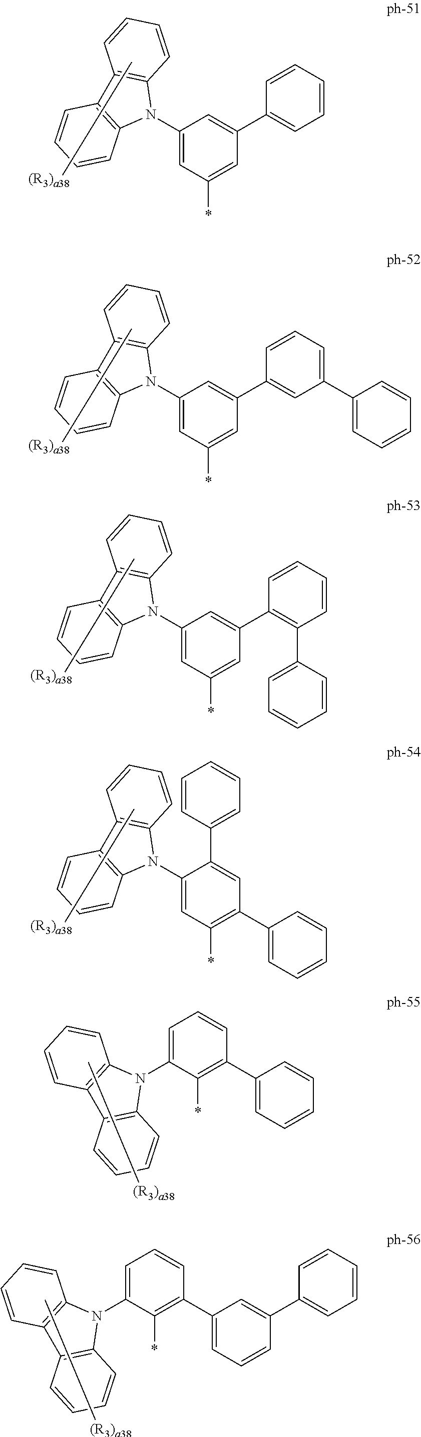

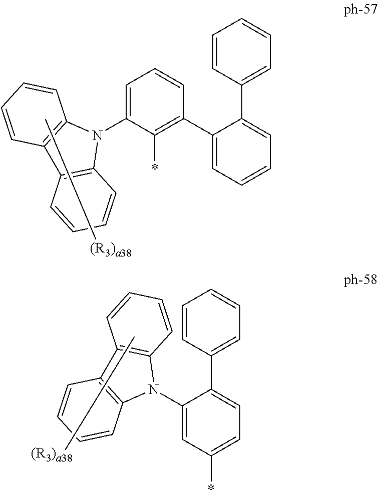

wherein, in Formulae ph-51 to ph-58,
R₃ is as described herein,
a38 may be an integer from 0 to 8, and
* indicates a binding site to a neighboring atom.

In one or more embodiments, the first compound may be represented by Formula 1-1:

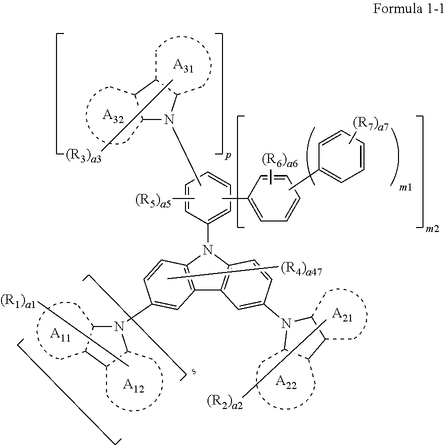

wherein, in Formula 1-1,
rings A₁₁ to A₃₂, R₁ to R₅, a1 to a3, a5, p, s, m1, and m2 are each as described herein, and
a47 may be an integer from 0 to 7.

In one or more embodiments, the first compound may satisfy at least one of Conditions 1 to 4:

### Condition 1

the number of carbazole groups in the first compound is 3 or more

### Condition 2

the number of benzene groups linked to each other only via a carbon-carbon single bond in the first compound is 2 or more

### Condition 3

the first compound includes at least one deuterium

### Condition 4

the first compound includes at least one tert-butyl group

The second compound may include a triazine group.

For example, the second compound may include:
a triazine group; and
at least one carbazole group, at least one silicon-containing group, or a combination thereof.

Examples of the silicon-containing group may include a group represented by *—Si(Ar₁₁)(Ar₁₂) (Ar₁₃).

In an embodiment, the second compound may be a compound represented by Formula 2:

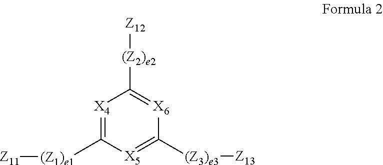

wherein, in Formula 2,
X₄ may be N, C(H), or C(Z₁₄), X₅ may be N, C(H), or C(Z₁₅), X₆ may be N, C(H), or C(Z₁₆), wherein at least one of X₄ to X₆ is N,
Z₁ to Z₃ may each independently be *—Si(Ar₁)(Ar₂)—*′, a C₅-C₃₀ carbocyclic group unsubstituted or substituted with at least one R₀, or a C₂-C₃₀ heterocyclic group unsubstituted or substituted with at least one R₀,
e1 to e3 may each independently be 0, 1, 2, or 3, i) when e1 is 0, a group represented by *—(Z₁)ₑ₁—*′ may be a single bond, ii) when e2 is 0, a group represented by *—(Z₂)ₑ₂—*′ may be a single bond, and i) when e3 is 0, a group represented by *—(Z₃)ₑ₃—*′ may be a single bond,
Z₁₁ to Z₁₃ may each independently be *—Si(Ar₁₁)(Ar₁₂)(Ar₁₃), a C₅-C₃₀ carbocyclic group unsubstituted or substituted with at least one R₀, or a C₂-C₃₀ heterocyclic group unsubstituted or substituted with at least one R₀,
Ar₁, Ar₂, and Ar₁₁ to Ar₁₃ may each independently be a C₅-C₃₀ carbocyclic group unsubstituted or substituted with at least one R₀ or a C₂-C₃₀ heterocyclic group unsubstituted or substituted with at least one R₀, and
R₀ and Z₁₄ to Z₁₆ may each independently be:
deuterium, a C₁-C₆₀ alkyl group, or a deuterated C₁-C₆₀ alkyl group; or
a C₅-C₃₀ carbocyclic group or a C₂-C₃₀ heterocyclic group, each unsubstituted or substituted with deuterium, a C₁-C₆₀ alkyl group, a deuterated C₁-C₆₀ alkyl group, a C₅-C₃₀ carbocyclic group, a C₂-C₃₀ heterocyclic group, or a combination thereof.

For example, at least two of X₄ to X₅ in Formula 2 may be N.

In an embodiment, X₄ to X₅ in Formula 2 may all be N.

In an embodiment, Z₁ to Z₃ may each independently be *—Si(Ar₁)(Ar₂)—*′, a benzene group unsubstituted or substituted with at least one R₀, or a carbazole group unsubstituted or substituted with at least one R₀.

In one or more embodiments, Z₁ to Z₃ may each independently be a group represented by one of Formulae 2(1) to 2(3):

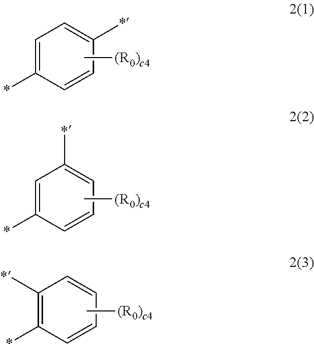

wherein, in Formulae 2(1) to 2(3),
R₀ is as described herein,
c4 may be an integer from 0 to 4, and
* and *′ each indicate a binding site to a neighboring atom.

In one or more embodiments, Z₁ to Z₃ may each independently be a group represented by one of Formulae 2(2) and 2(3).

In one or more embodiments, in Formula 2,
i) e1 may be 1, 2, or 3, and e2 and e3 may each be 0,
ii) e1 and e2 may each independently be 1, 2, or 3, and e3 may be 0, or
iii) e1, e2, and e3 may each independently be 1, 2, or 3.

In one or more embodiments, Z₁₁ to Z₁₃ may each independently be:
—Si(Ar₁₁)(Ar₁₂)(Ar₁₃); or
a phenyl group or an N-carbazolyl group, each unsubstituted or substituted with deuterium, a C₁-C₂₀ alkyl group, a phenyl group, an N-carbazolyl group, or a combination thereof.

In one or more embodiments, at least one of Z₁₁ to Z₁₃ may each independently be:
—Si(Ar₁₁)(Ar₁₂)(Ar₁₃); or
an N-carbazolyl group unsubstituted or substituted with deuterium, a C₁-C₂₀ alkyl group, a phenyl group, an N-carbazolyl group, or a combination thereof.

In one or more embodiments, at least one of Z₁₁ to Z₁₃ may be *—Si(Ar₁₁)(Ar₁₂)(Ar₁₃) or a group represented by Formula 2-CZ:

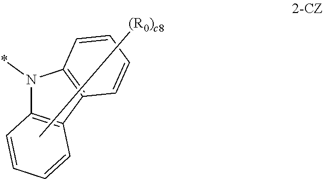

wherein, in Formula 2-CZ,
R₀ is as described herein,
c8 may be an integer from 0 to 8, and
* indicates a binding site to a neighboring atom.

In one or more embodiments, in Formula 2,
i) Z₁₁, Z₁₂, and Z₁₃ may each be identical to each other,
ii) Z₁₁ and Z₁₂ may be different from each other and Z₁₂ and Z₁₃ may be identical to each other, or
iii) Z₁₁, Z₁₂, and Z₁₃ may each be different from each other.

In one or more embodiments, Ar₁, Ar₂, and Ar₁₁ to Ar₁₃ may each independently be a phenyl group unsubstituted or substituted with deuterium, a C₁-C₂₀ alkyl group, a phenyl group, an N-carbazolyl group, or a combination thereof.

In one or more embodiments, R₀ and Z₁₄ to Z₁₆ may each independently be:
deuterium, a C₁-C₂₀ alkyl group, or a deuterated C₁-C₂₀ alkyl group; or
a phenyl group or an N-carbazolyl group, each unsubstituted or substituted with deuterium, a C₁-C₂₀ alkyl group, a phenyl group, an N-carbazolyl group, or a combination thereof.

In one or more embodiments, the second compound may be a compound represented by Formula 2-1 or 2-2:

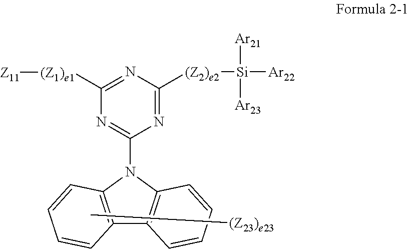

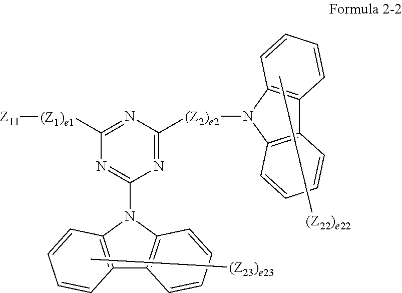

wherein, in Formulae 2-1 and 2-2,
Z₁, Z₂, e1, e2, and Z₁₁ may each be as described herein,
Z₂₂ and Z₂₃ may each be as described for R₀,
e23 and e22 may each independently be an integer from 0 to 8, and
Ar₂₁, Ar₂₂, and Ar₂₃ may be as described for Ar₁₁, Ar₁₂, and Ar₁₃, respectively.

In one or more embodiments, e2 in Formula 2-1 may not be 0.

In one or more embodiments, the sum of e1 and e2 may be at least 1. In some embodiments, e1 may be 1 and e2 may be 0. In some embodiments, e1 may be 0 and e2 may be 1. In some embodiments, e1 may be 1 and e2 may be 1.

In one or more embodiments, the second compound may be represented by Formula 2-1 or 2-2 and may satisfy at least one of Conditions 11 to 13:

### Condition 11

Z₁₁ in Formulae 2-1 and 2-2 are each a phenyl group or an N-carbazolyl group, each unsubstituted or substituted with deuterium, a C₁-C₂₀ alkyl group, a phenyl group, an N-carbazolyl group, or a combination thereof

### Condition 12

Z₁₁ in Formulae 2-1 and 2-2 is *—Si(Ar₁₁)(Ar₁₂)(Ar₁₃)

### Condition 13

Z₁ and Z₂ in Formulae 2-1 and 2-2 are each independently an o-phenylene group or a m-phenylene group, each unsubstituted or substituted with deuterium, a C₁-C₂₀ alkyl group, a phenyl group, an N-carbazolyl group, or a combination thereof.

In one or more embodiments, the second compound may satisfy at least one of Conditions 14 and 15:

### Condition 14

the second compound includes at least one deuterium

### Condition 15

the second compound includes at least one tert-butyl group.

In an embodiment, when at least one of the first compound and second compound includes at least one deuterium, the lifespan characteristics of the light-emitting device including the composition may be improved.

In one or more embodiments, the triplet (T₁) energy level of the first compound may be in a range of about 2.8 electron volt (eV) or greater, about 2.8 eV to about 3.5 eV, about 2.8 eV to about 3.3 eV, or about 2.8 eV to about 3.1 eV.

In one or more embodiments, the triplet (T₁) energy level of the second compound may be in a range of about 2.3 eV to about 3.5 eV, about 2.4 eV to about 3.2 eV, about 2.5 eV to about 3.1 eV, or about 2.6 eV to about 3.0 eV.

The triplet (T₁) energy level may be evaluated by the density functional theory (DFT), for example, the DFT method of the Gaussian program. For example, the evaluation method of the triplet energy level may be understood by referring to the following Evaluation Example 1.

If the triplet energy level range of the first compound satisfies the above range, the light-emitting device including the composition may have excellent color purity, luminescence efficiency, and/or lifespan characteristics. For example, the light-emitting device may emit blue light having excellent color purity in addition to having excellent luminescence efficiency and/or lifespan characteristics.

The weight ratio of the first compound and the second compound in the composition may be in a range of about 1:99 to about 99:1, about 10:90 to about 90:10, about 20:80 to about 80:20, about 30:70 to about 70:30, or about 40:60 to about 60:40. When the weight ratio of the first compound and the second compound satisfies the above range, a balance between holes and electrons in the emission layer using the composition may be effectively achieved, and thus, a light-emitting device having excellent luminescence efficiency and/or lifespan characteristics may be implemented.

The “first compound” as described herein may refer to a single compound represented by Formula 1 or a combination of two or more different compounds represented by Formula 1.

The “second compound” as described herein may refer to a single compound represented by Formula 2 (for example, Formulae 2-1 and 2-2) or a combination of two or more different compounds represented by Formula 2 (for example, Formulae 2-1 and 2-2).

In an embodiment, the first compound may be, for example, one of Compounds H1 to H66, a compound in which at least one hydrogen is substituted with deuterium from among Compounds H2, H3, and H6 to H16, or a combination thereof:

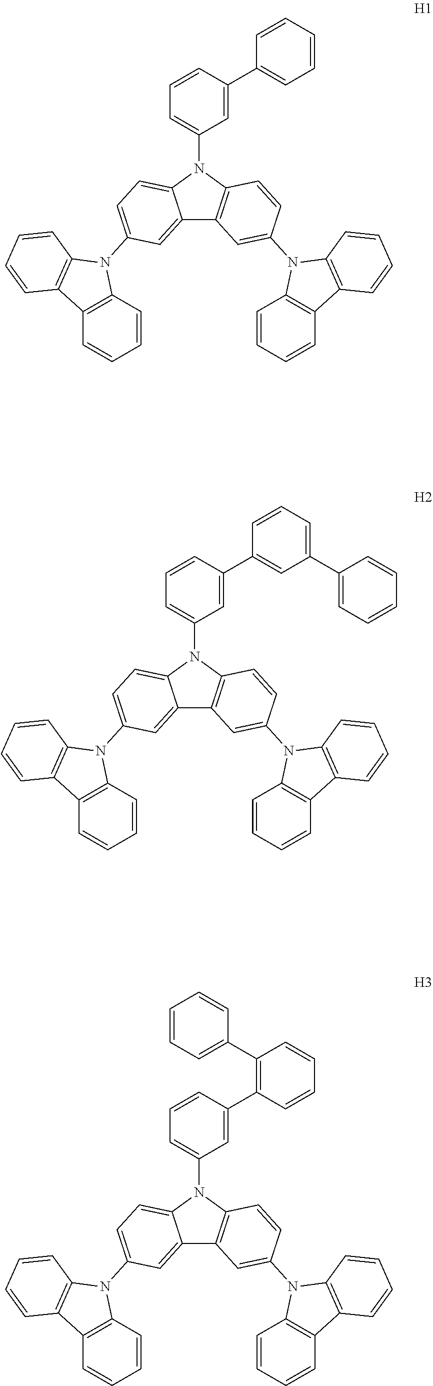 [SMILES: c1ccc(-c2cccc(-n3c4ccc(-n5c6ccccc6c6ccccc65)cc4c4cc(-n5c6ccccc6c6ccccc65)ccc43)c2)cc1 ; c1ccc(-c2cccc(-c3cccc(-n4c5ccc(-n6c7ccccc7c7ccccc76)cc5c5cc(-n6c7ccccc7c7ccccc76)ccc54)c3)c2)cc1 ; c1ccc(-c2ccccc2-c2cccc(-n3c4ccc(-n5c6ccccc6c6ccccc65)cc4c4cc(-n5c6ccccc6c6ccccc65)ccc43)c2)cc1]

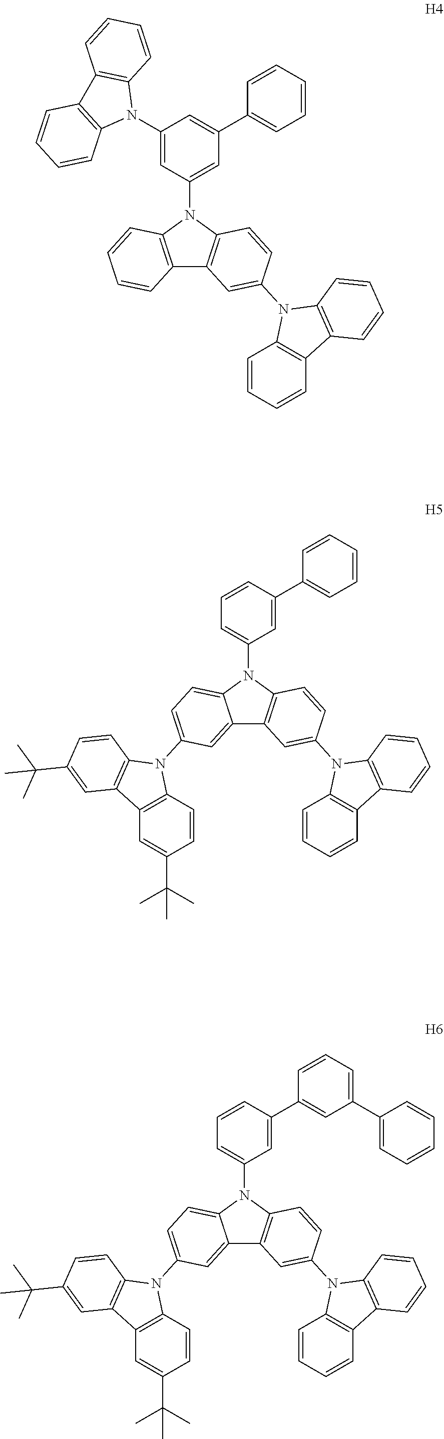 [SMILES: c1ccc(-c2cc(-n3c4ccccc4c4ccccc43)cc(-n3c4ccccc4c4cc(-n5c6ccccc6c6ccccc65)ccc43)c2)cc1 ; CC(C)(C)c1ccc2c(c1)c1cc(C(C)(C)C)ccc1n2-c1ccc2c(c1)c1cc(-n3c4ccccc4c4ccccc43)ccc1n2-c1cccc(-c2ccccc2)c1 ; CC(C)(C)c1ccc2c(c1)c1cc(C(C)(C)C)ccc1n2-c1ccc2c(c1)c1cc(-n3c4ccccc4c4ccccc43)ccc1n2-c1cccc(-c2cccc(-c3ccccc3)c2)c1]

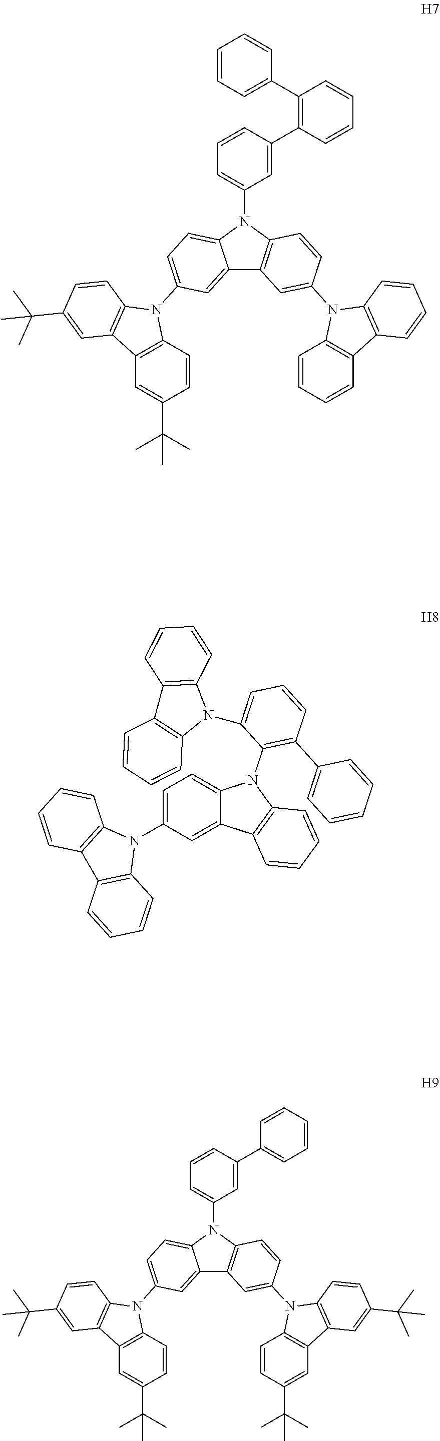 [SMILES: CC(C)(C)c1ccc2c(c1)c1cc(C(C)(C)C)ccc1n2-c1ccc2c(c1)c1cc(-n3c4ccccc4c4ccccc43)ccc1n2-c1cccc(-c2ccccc2-c2ccccc2)c1 ; c1ccc(-c2cccc(-n3c4ccccc4c4ccccc43)c2-n2c3ccccc3c3cc(-n4c5ccccc5c5ccccc54)ccc32)cc1 ; CC(C)(C)c1ccc2c(c1)c1cc(C(C)(C)C)ccc1n2-c1ccc2c(c1)c1cc(-n3c4ccc(C(C)(C)C)cc4c4cc(C(C)(C)C)ccc43)ccc1n2-c1cccc(-c2ccccc2)c1]

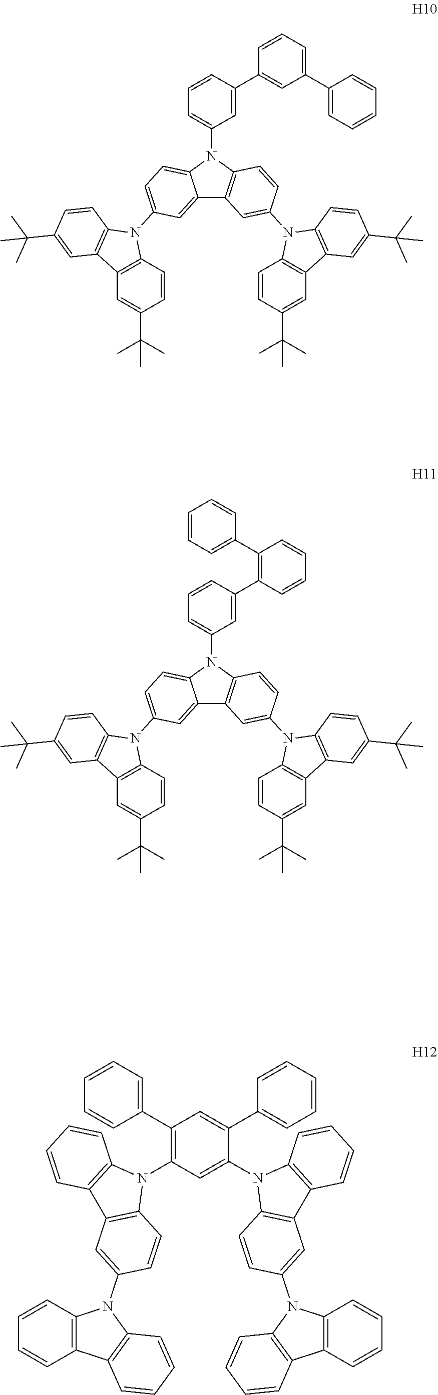

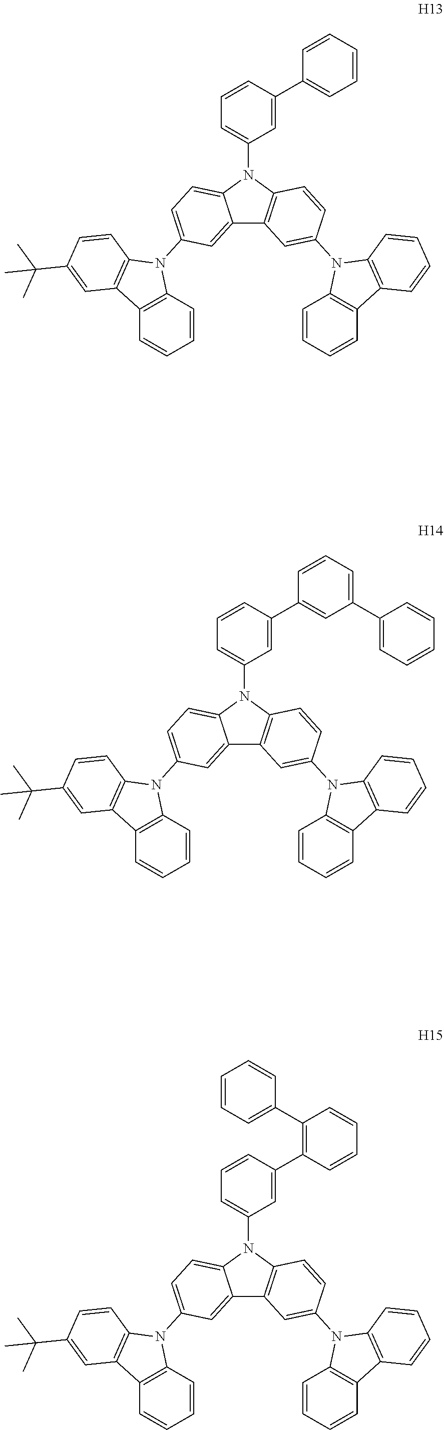 [SMILES: CC(C)(C)c1ccc2c(c1)c1ccccc1n2-c1ccc2c(c1)c1cc(-n3c4ccccc4c4ccccc43)ccc1n2-c1cccc(-c2ccccc2)c1 ; CC(C)(C)c1ccc2c(c1)c1ccccc1n2-c1ccc2c(c1)c1cc(-n3c4ccccc4c4ccccc43)ccc1n2-c1cccc(-c2cccc(-c3ccccc3)c2)c1 ; CC(C)(C)c1ccc2c(c1)c1ccccc1n2-c1ccc2c(c1)c1cc(-n3c4ccccc4c4ccccc43)ccc1n2-c1cccc(-c2ccccc2-c2ccccc2)c1]

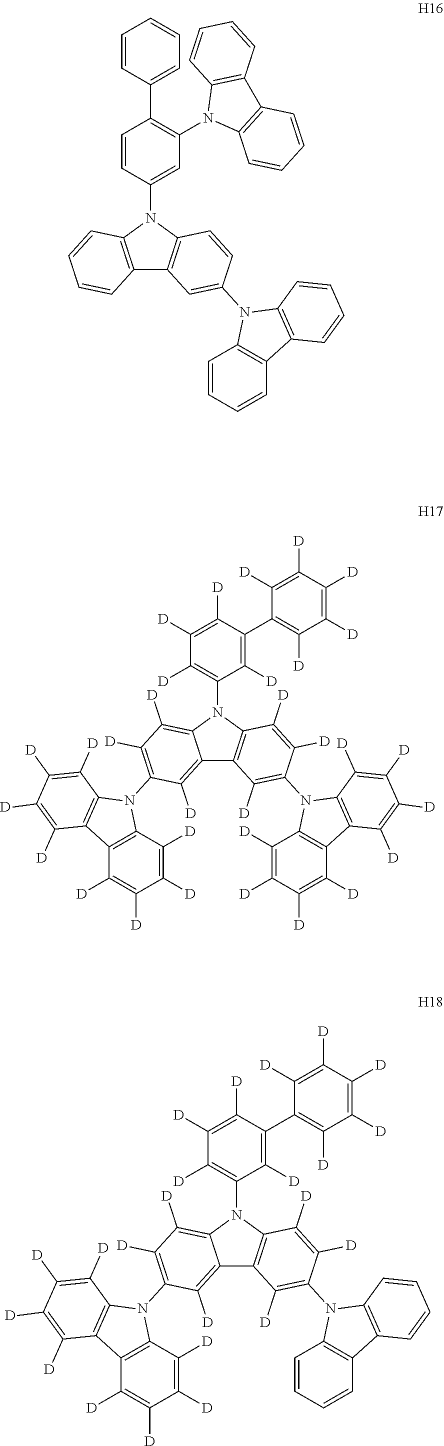

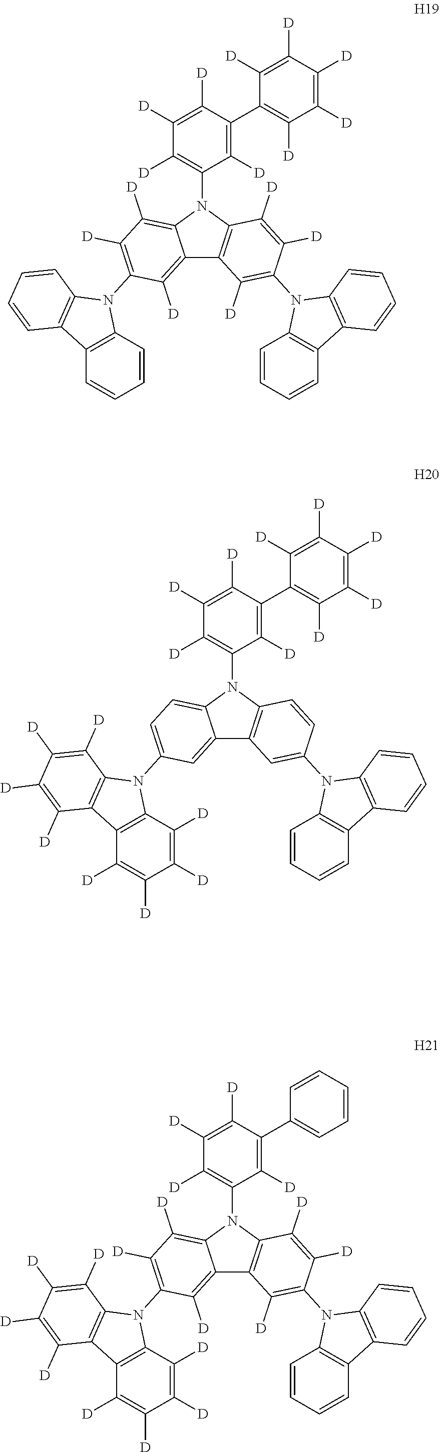

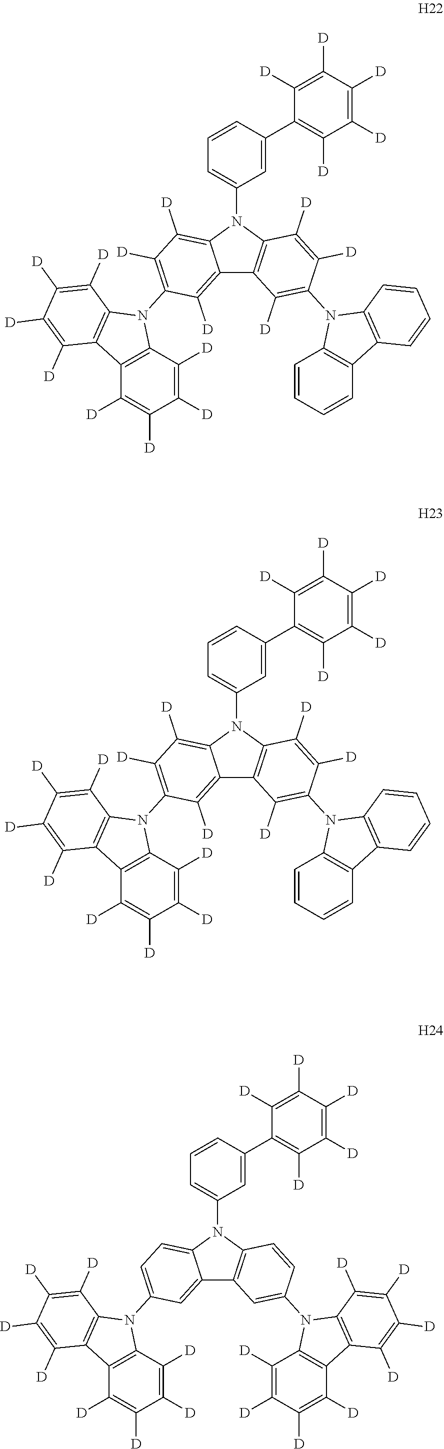

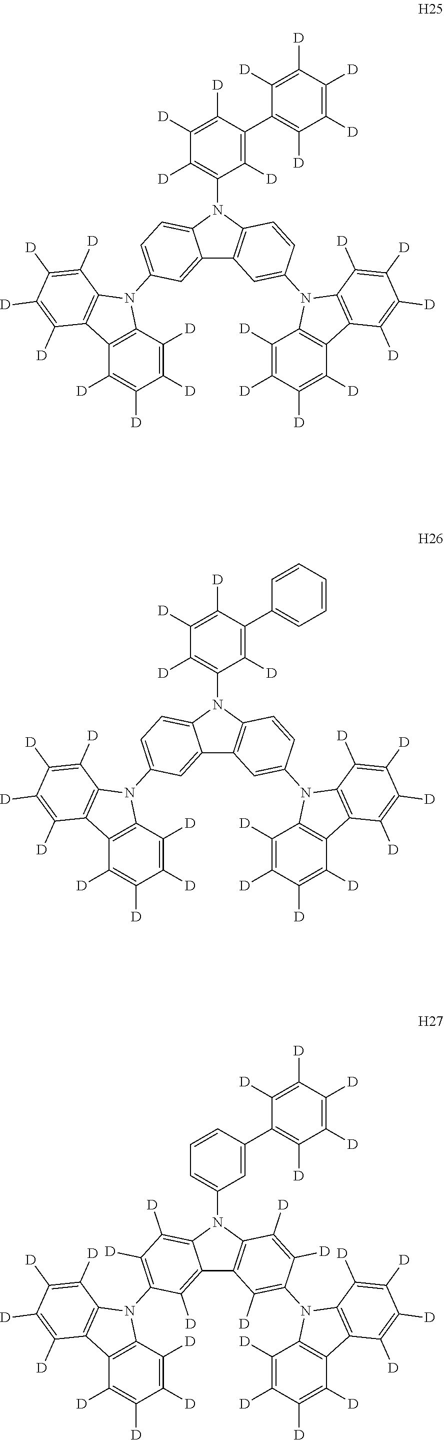

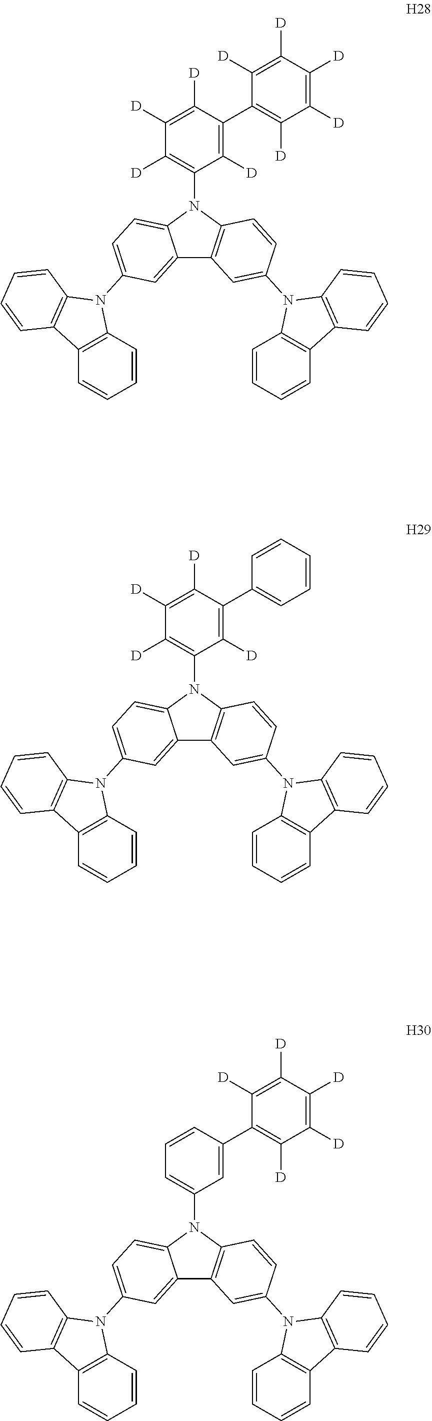 [SMILES: [2H]c1c([2H])c([2H])c(-c2c([2H])c([2H])c([2H])c(-n3c4ccc(-n5c6ccccc6c6ccccc65)cc4c4cc(-n5c6ccccc6c6ccccc65)ccc43)c2[2H])c([2H])c1[2H] ; [2H]c1c([2H])c(-c2ccccc2)c([2H])c(-n2c3ccc(-n4c5ccccc5c5ccccc54)cc3c3cc(-n4c5ccccc5c5ccccc54)ccc32)c1[2H] ; [2H]c1c([2H])c([2H])c(-c2cccc(-n3c4ccc(-n5c6ccccc6c6ccccc65)cc4c4cc(-n5c6ccccc6c6ccccc65)ccc43)c2)c([2H])c1[2H]]

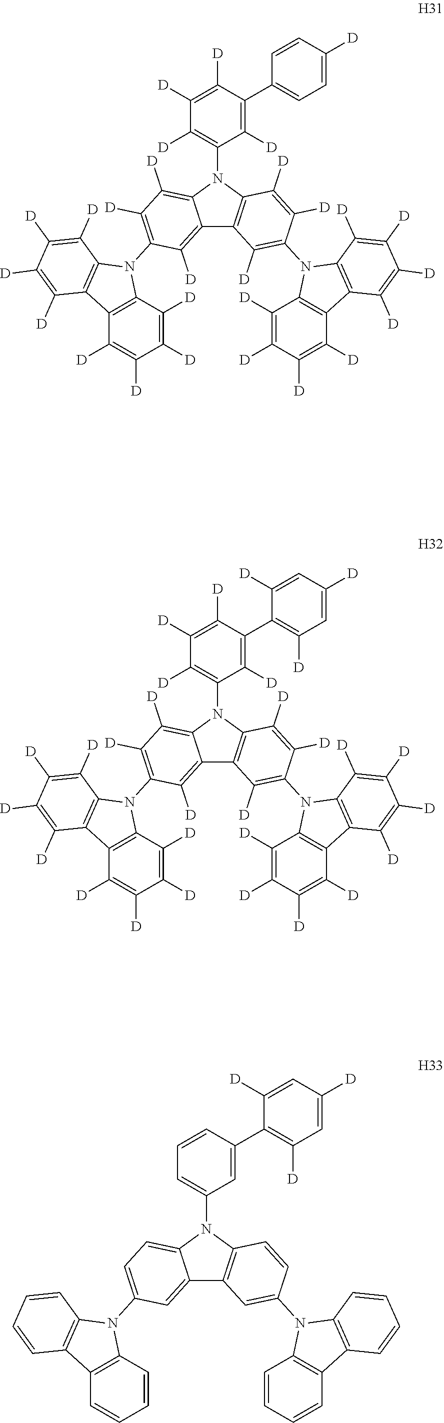

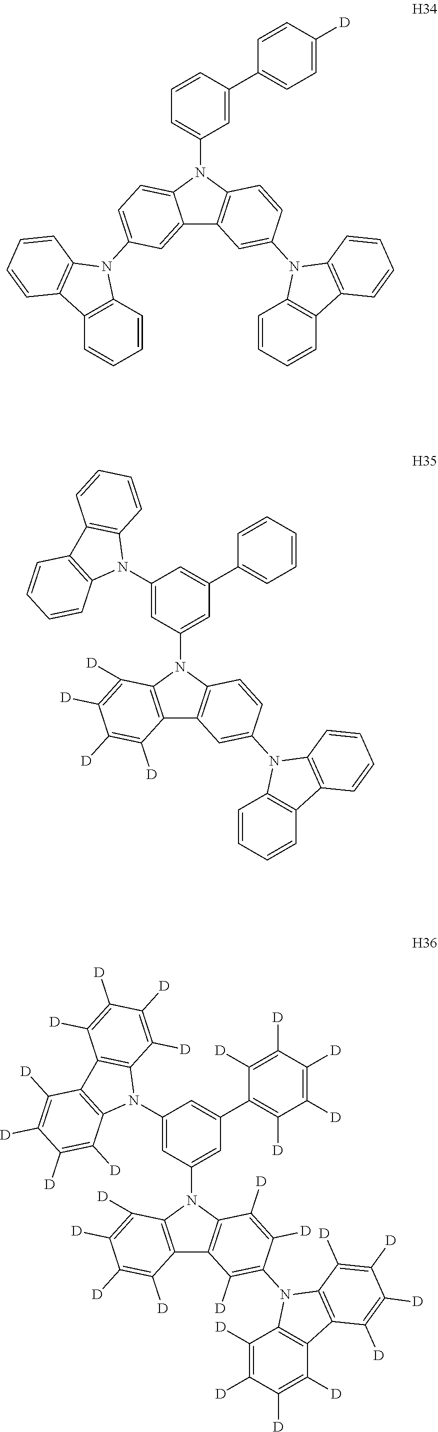 [SMILES: [2H]c1ccc(-c2cccc(-n3c4ccc(-n5c6ccccc6c6ccccc65)cc4c4cc(-n5c6ccccc6c6ccccc65)ccc43)c2)cc1 ; [2H]c1c([2H])c([2H])c2c(c1[2H])c1cc(-n3c4ccccc4c4ccccc43)ccc1n2-c1cc(-c2ccccc2)cc(-n2c3ccccc3c3ccccc32)c1 ; [2H]c1c([2H])c([2H])c(-c2cc(-n3c4c([2H])c([2H])c([2H])c([2H])c4c4c([2H])c([2H])c([2H])c([2H])c43)cc(-n3c4c([2H])c([2H])c([2H])c([2H])c4c4c([2H])c(-n5c6c([2H])c([2H])c([2H])c([2H])c6c6c([2H])c([2H])c([2H])c([2H])c65)c([2H])c([2H])c43)c2)c([2H])c1[2H]]

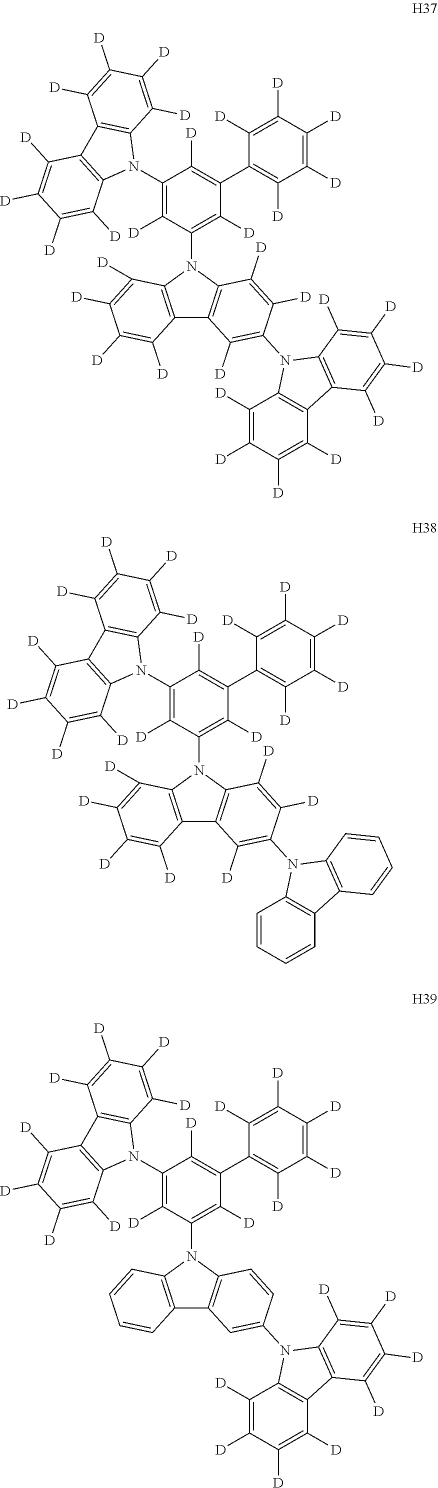

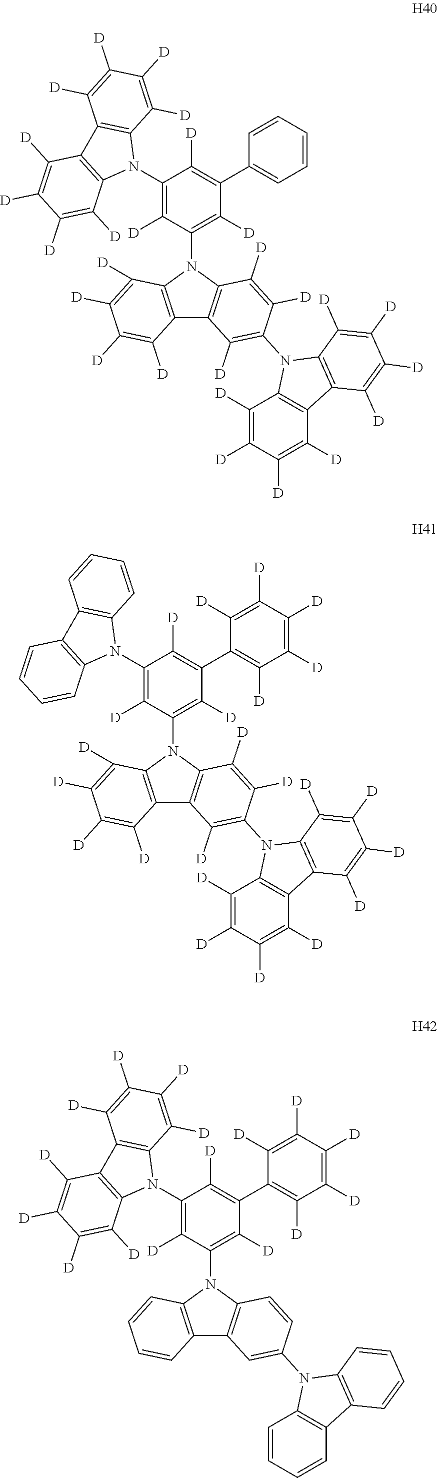

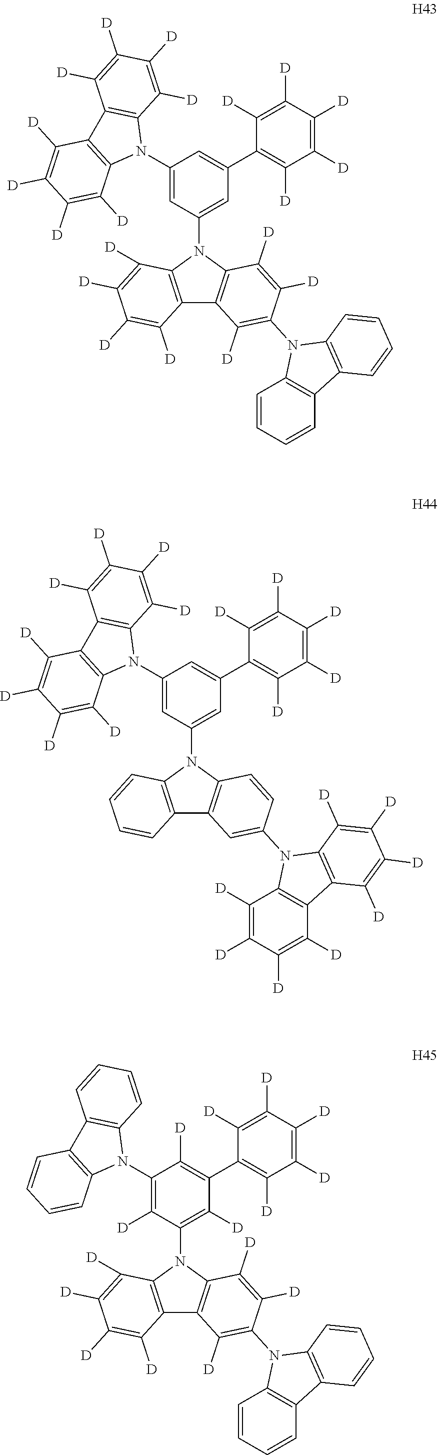

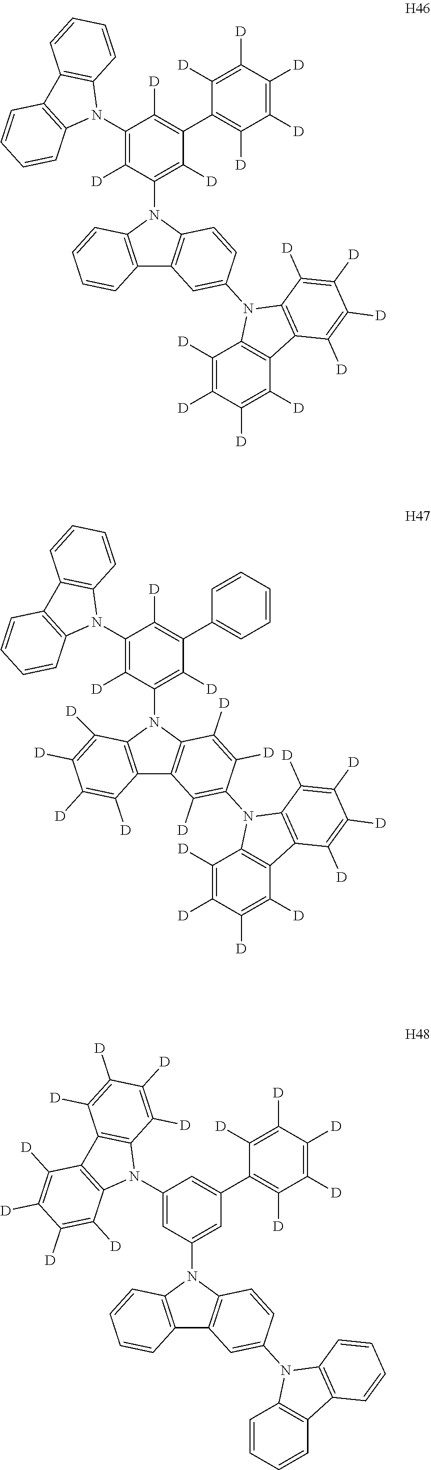

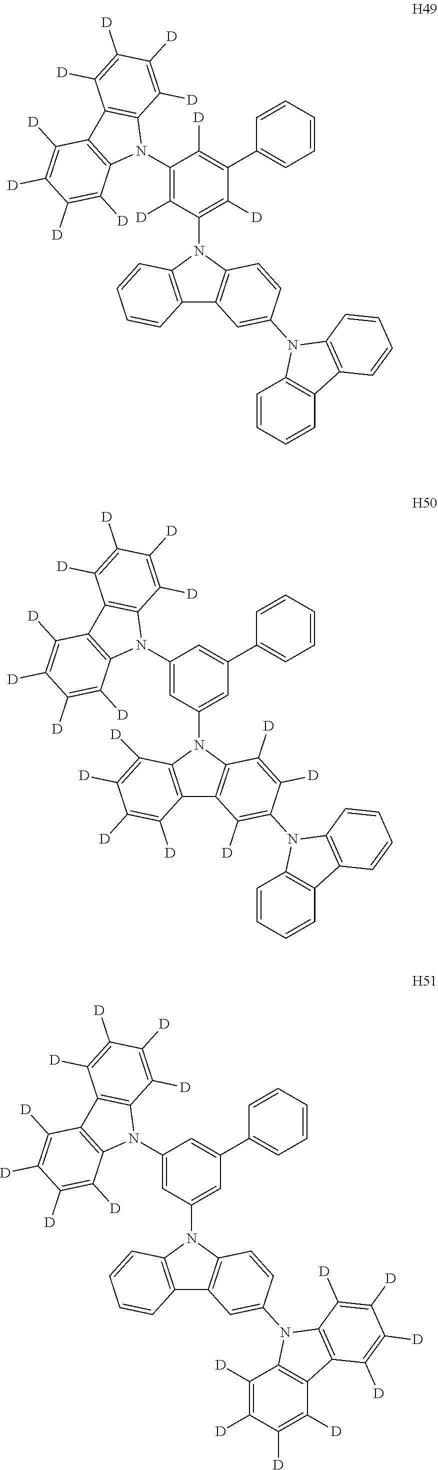

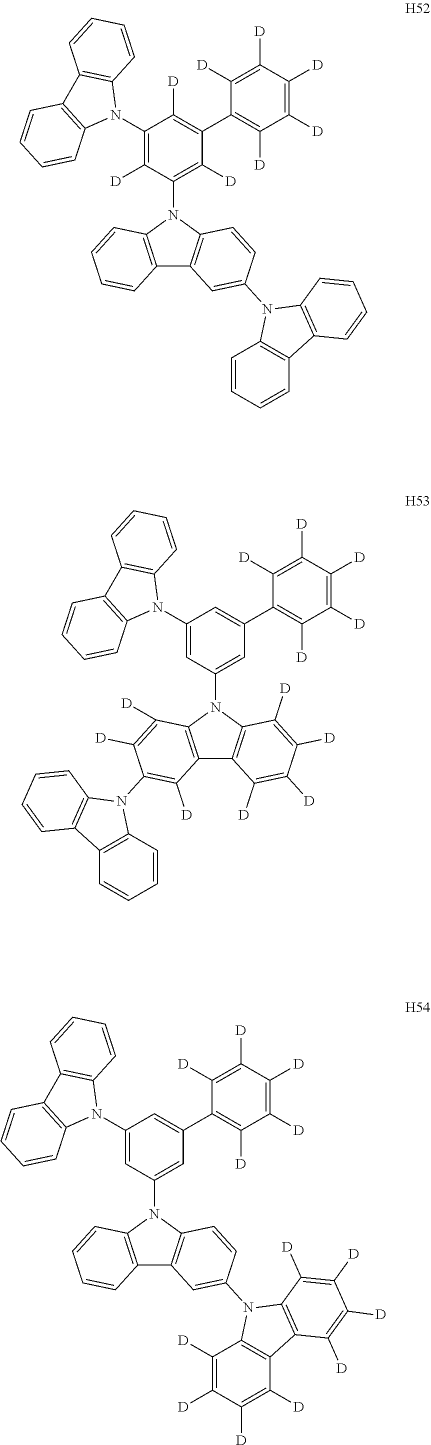 [SMILES: [2H]c1c([2H])c([2H])c(-c2c([2H])c(-n3c4ccccc4c4ccccc43)c([2H])c(-n3c4ccccc4c4cc(-n5c6ccccc6c6ccccc65)ccc43)c2[2H])c([2H])c1[2H] ; [2H]c1c([2H])c([2H])c(-c2cc(-n3c4ccccc4c4ccccc43)cc(-n3c4c([2H])c([2H])c([2H])c([2H])c4c4c([2H])c(-n5c6ccccc6c6ccccc65)c([2H])c([2H])c43)c2)c([2H])c1[2H] ; [2H]c1c([2H])c([2H])c(-c2cc(-n3c4ccccc4c4ccccc43)cc(-n3c4ccccc4c4cc(-n5c6c([2H])c([2H])c([2H])c([2H])c6c6c([2H])c([2H])c([2H])c([2H])c65)ccc43)c2)c([2H])c1[2H]]

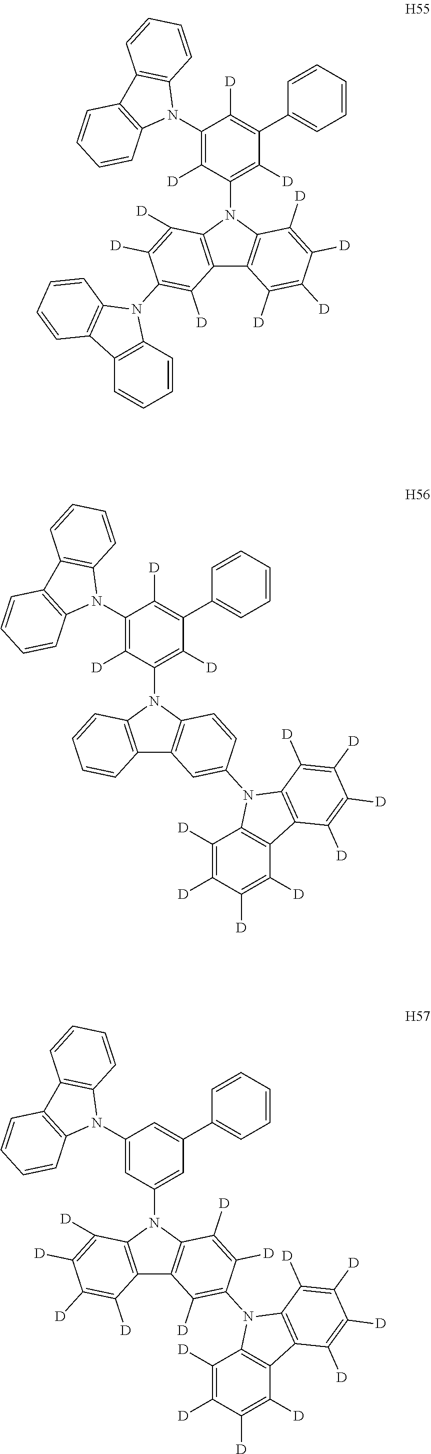 [SMILES: [2H]c1c(-c2ccccc2)c([2H])c(-n2c3c([2H])c([2H])c([2H])c([2H])c3c3c([2H])c(-n4c5ccccc5c5ccccc54)c([2H])c([2H])c32)c([2H])c1-n1c2ccccc2c2ccccc21 ; [2H]c1c(-c2ccccc2)c([2H])c(-n2c3ccccc3c3cc(-n4c5c([2H])c([2H])c([2H])c([2H])c5c5c([2H])c([2H])c([2H])c([2H])c54)ccc32)c([2H])c1-n1c2ccccc2c2ccccc21 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccccc2)cc(-n2c3ccccc3c3ccccc32)c1]

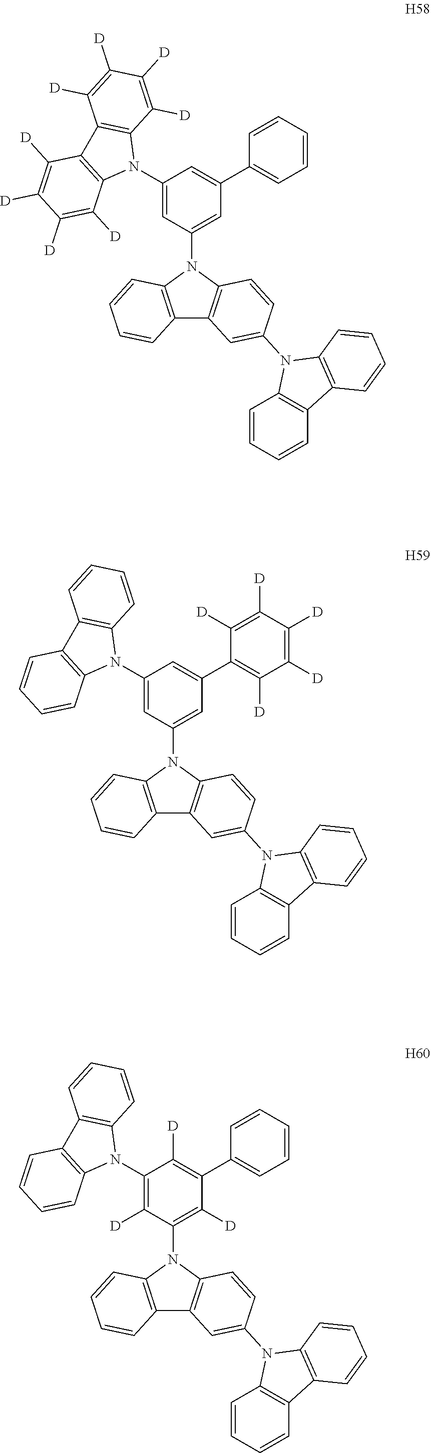 [SMILES: [2H]c1c([2H])c([2H])c2c(c1[2H])c1c([2H])c([2H])c([2H])c([2H])c1n2-c1cc(-c2ccccc2)cc(-n2c3ccccc3c3cc(-n4c5ccccc5c5ccccc54)ccc32)c1 ; [2H]c1c([2H])c([2H])c(-c2cc(-n3c4ccccc4c4ccccc43)cc(-n3c4ccccc4c4cc(-n5c6ccccc6c6ccccc65)ccc43)c2)c([2H])c1[2H] ; [2H]c1c(-c2ccccc2)c([2H])c(-n2c3ccccc3c3cc(-n4c5ccccc5c5ccccc54)ccc32)c([2H])c1-n1c2ccccc2c2ccccc21]

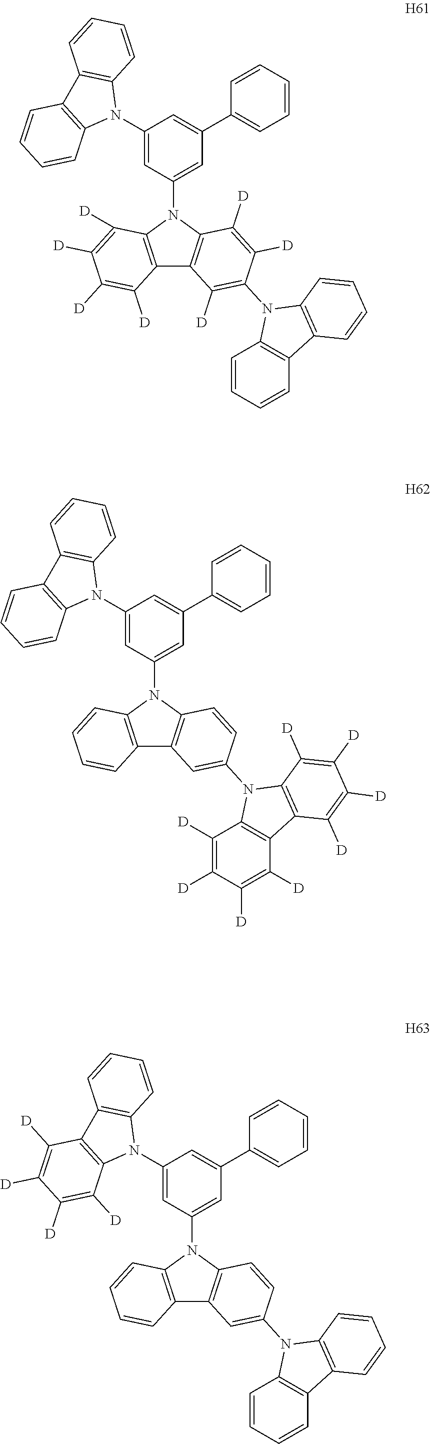 [SMILES: [2H]c1c([2H])c([2H])c2c(c1[2H])c1c([2H])c(-n3c4ccccc4c4ccccc43)c([2H])c([2H])c1n2-c1cc(-c2ccccc2)cc(-n2c3ccccc3c3ccccc32)c1 ; [2H]c1c([2H])c([2H])c2c(c1[2H])c1c([2H])c([2H])c([2H])c([2H])c1n2-c1ccc2c(c1)c1ccccc1n2-c1cc(-c2ccccc2)cc(-n2c3ccccc3c3ccccc32)c1 ; [2H]c1c([2H])c([2H])c2c(c1[2H])c1ccccc1n2-c1cc(-c2ccccc2)cc(-n2c3ccccc3c3cc(-n4c5ccccc5c5ccccc54)ccc32)c1]

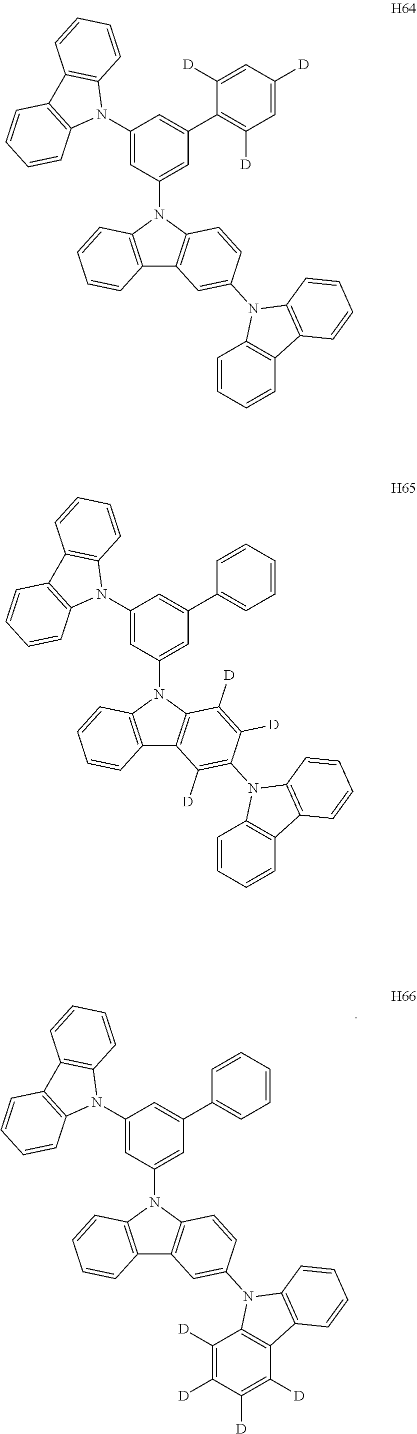 [SMILES: [2H]c1cc([2H])c(-c2cc(-n3c4ccccc4c4ccccc43)cc(-n3c4ccccc4c4cc(-n5c6ccccc6c6ccccc65)ccc43)c2)c([2H])c1 ; [2H]c1c(-n2c3ccccc3c3ccccc32)c([2H])c2c3ccccc3n(-c3cc(-c4ccccc4)cc(-n4c5ccccc5c5ccccc54)c3)c2c1[2H] ; [2H]c1c([2H])c([2H])c2c(c1[2H])c1ccccc1n2-c1ccc2c(c1)c1ccccc1n2-c1cc(-c2ccccc2)cc(-n2c3ccccc3c3ccccc32)c1]

In one or more embodiments, the second compound may be one of Compounds E1 to E47, a compound from which deuterium is replaced with a hydrogen from among Compounds E19 to E23, E25, E26, E28, E30, E31, and E35 to E43, a compound of which the substitution location of deuterium and/or the number of deuterium is changed from among Compounds E19 to E23, E25, E26, E28, E30, E31, and E35 to E43, a compound in which at least one hydrogen is substituted with deuterium from among Compounds E1 to E18, E24, E27, E29, E32 to E34, and E44 to E47, or a combination thereof:

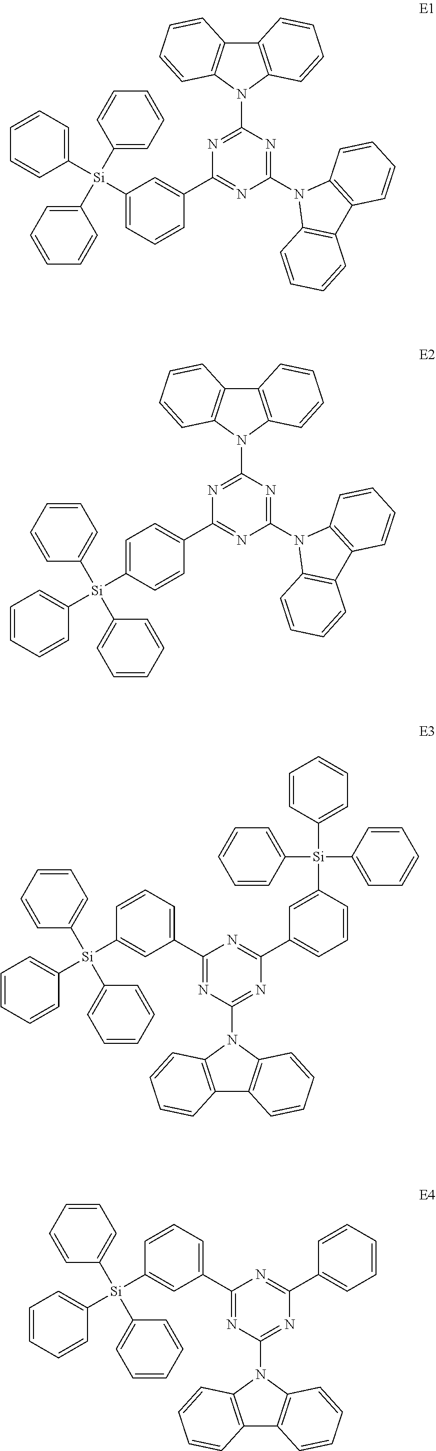 [SMILES: c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1 ; c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc(-c2nc(-c3cccc([Si](c4ccccc4)(c4ccccc4)c4ccccc4)c3)nc(-n3c4ccccc4c4ccccc43)n2)cc1]

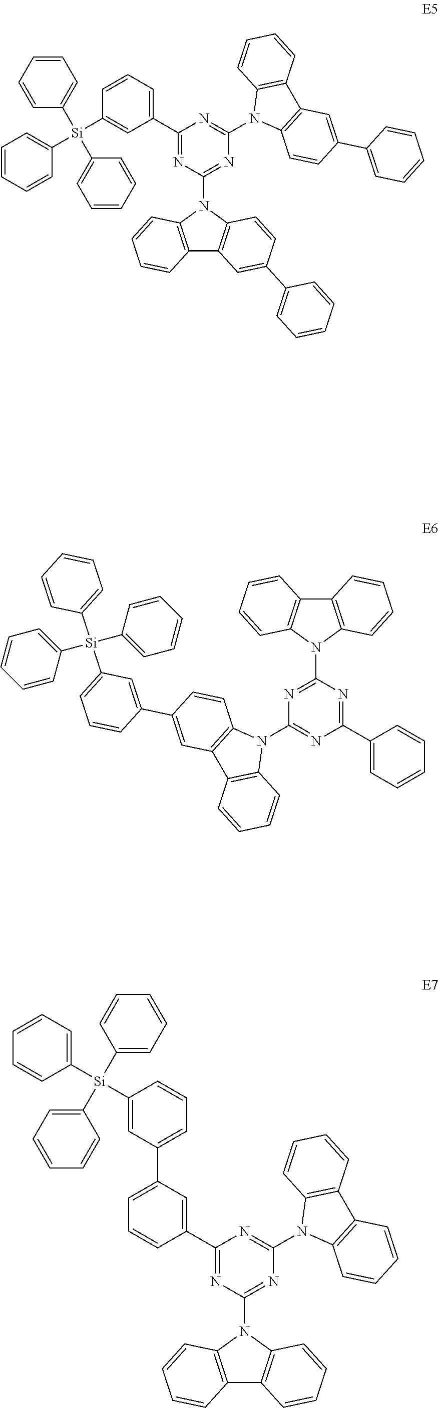 [SMILES: c1ccc(-c2ccc3c(c2)c2ccccc2n3-c2nc(-c3cccc([Si](c4ccccc4)(c4ccccc4)c4ccccc4)c3)nc(-n3c4ccccc4c4cc(-c5ccccc5)ccc43)n2)cc1 ; c1ccc(-c2nc(-n3c4ccccc4c4ccccc43)nc(-n3c4ccccc4c4cc(-c5cccc([Si](c6ccccc6)(c6ccccc6)c6ccccc6)c5)ccc43)n2)cc1 ; c1ccc([Si](c2ccccc2)(c2ccccc2)c2cccc(-c3cccc(-c4nc(-n5c6ccccc6c6ccccc65)nc(-n5c6ccccc6c6ccccc65)n4)c3)c2)cc1]

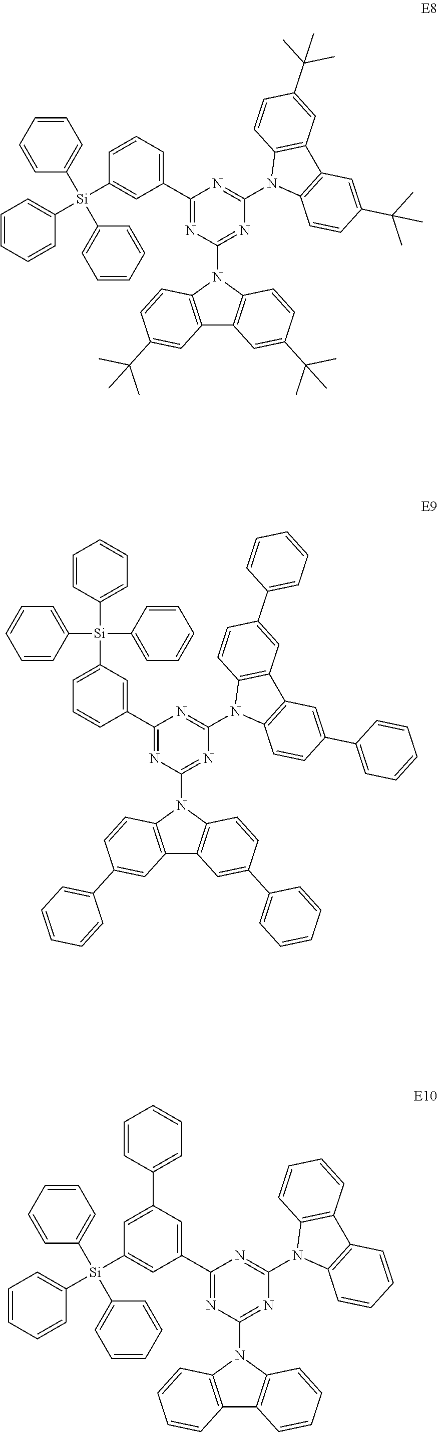 [SMILES: CC(C)(C)c1ccc2c(c1)c1cc(C(C)(C)C)ccc1n2-c1nc(-c2cccc([Si](c3ccccc3)(c3ccccc3)c3ccccc3)c2)nc(-n2c3ccc(C(C)(C)C)cc3c3cc(C(C)(C)C)ccc32)n1 ; c1ccc(-c2ccc3c(c2)c2cc(-c4ccccc4)ccc2n3-c2nc(-c3cccc([Si](c4ccccc4)(c4ccccc4)c4ccccc4)c3)nc(-n3c4ccc(-c5ccccc5)cc4c4cc(-c5ccccc5)ccc43)n2)cc1 ; c1ccc(-c2cc(-c3nc(-n4c5ccccc5c5ccccc54)nc(-n4c5ccccc5c5ccccc54)n3)cc([Si](c3ccccc3)(c3ccccc3)c3ccccc3)c2)cc1]

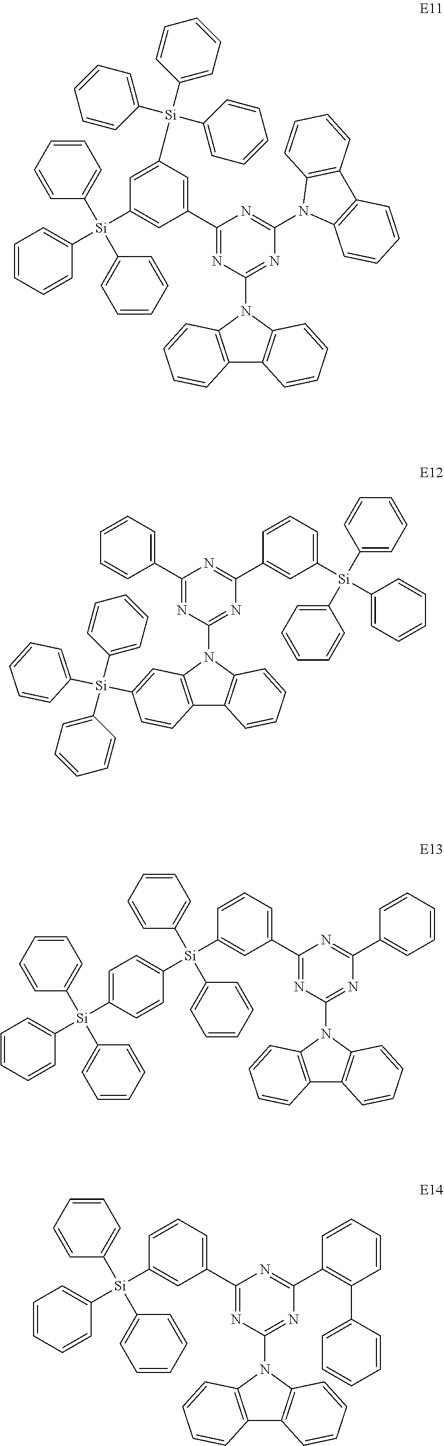 [SMILES: c1ccc([Si](c2ccccc2)(c2ccccc2)c2cc(-c3nc(-n4c5ccccc5c5ccccc54)nc(-n4c5ccccc5c5ccccc54)n3)cc([Si](c3ccccc3)(c3ccccc3)c3ccccc3)c2)cc1 ; c1ccc(-c2nc(-c3cccc([Si](c4ccccc4)(c4ccccc4)c4ccccc4)c3)nc(-n3c4ccccc4c4ccc([Si](c5ccccc5)(c5ccccc5)c5ccccc5)cc43)n2)cc1 ; c1ccc(-c2nc(-c3cccc([Si](c4ccccc4)(c4ccccc4)c4ccc([Si](c5ccccc5)(c5ccccc5)c5ccccc5)cc4)c3)nc(-n3c4ccccc4c4ccccc43)n2)cc1 ; c1ccc(-c2ccccc2-c2nc(-c3cccc([Si](c4ccccc4)(c4ccccc4)c4ccccc4)c3)nc(-n3c4ccccc4c4ccccc43)n2)cc1]

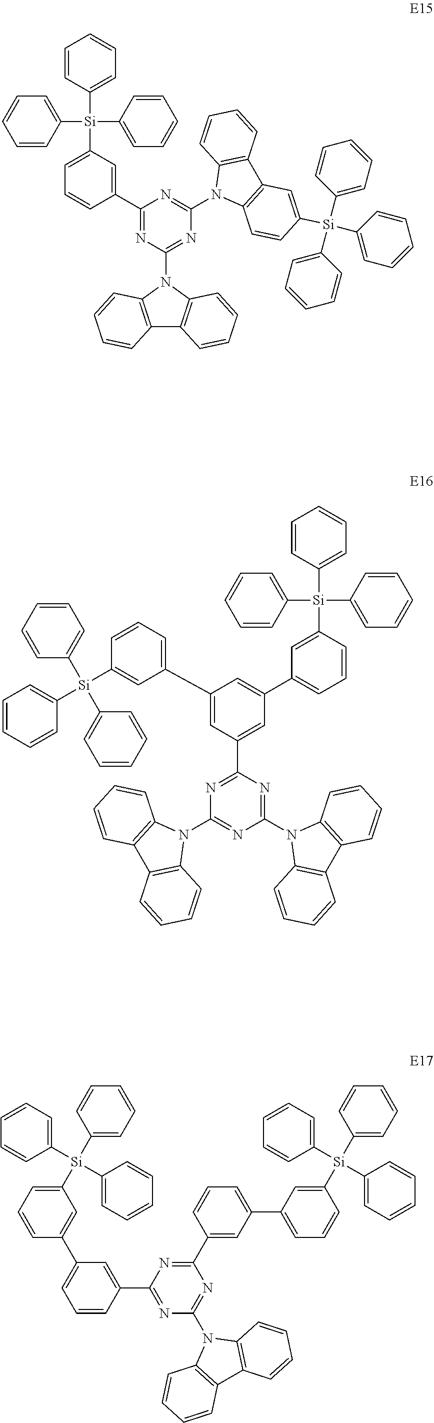 [SMILES: c1ccc([Si](c2ccccc2)(c2ccccc2)c2cccc(-c3nc(-n4c5ccccc5c5ccccc54)nc(-n4c5ccccc5c5cc([Si](c6ccccc6)(c6ccccc6)c6ccccc6)ccc54)n3)c2)cc1 ; c1ccc([Si](c2ccccc2)(c2ccccc2)c2cccc(-c3cc(-c4cccc([Si](c5ccccc5)(c5ccccc5)c5ccccc5)c4)cc(-c4nc(-n5c6ccccc6c6ccccc65)nc(-n5c6ccccc6c6ccccc65)n4)c3)c2)cc1 ; c1ccc([Si](c2ccccc2)(c2ccccc2)c2cccc(-c3cccc(-c4nc(-c5cccc(-c6cccc([Si](c7ccccc7)(c7ccccc7)c7ccccc7)c6)c5)nc(-n5c6ccccc6c6ccccc65)n4)c3)c2)cc1]

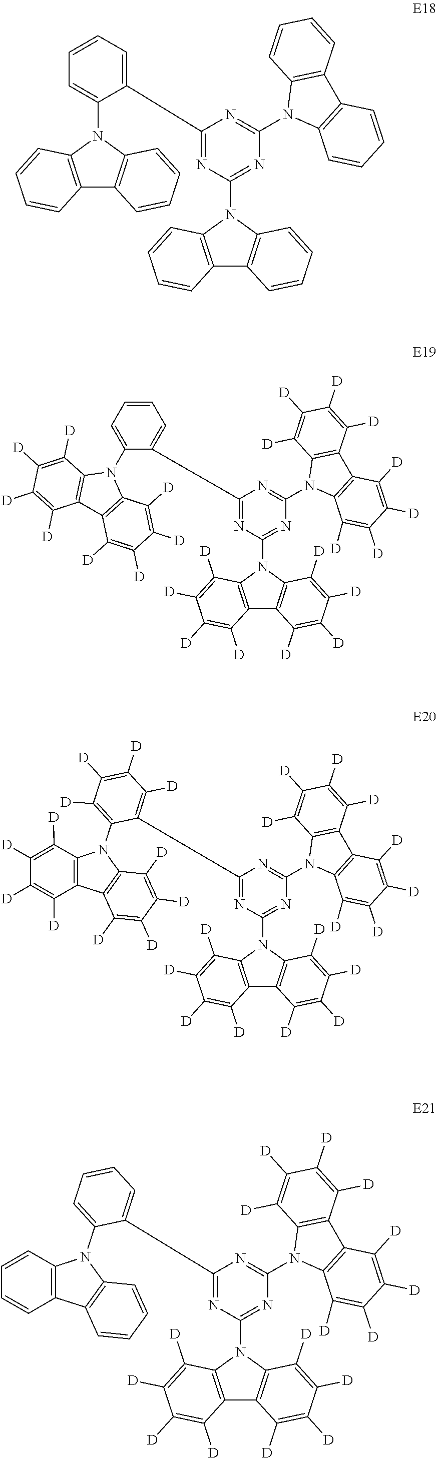

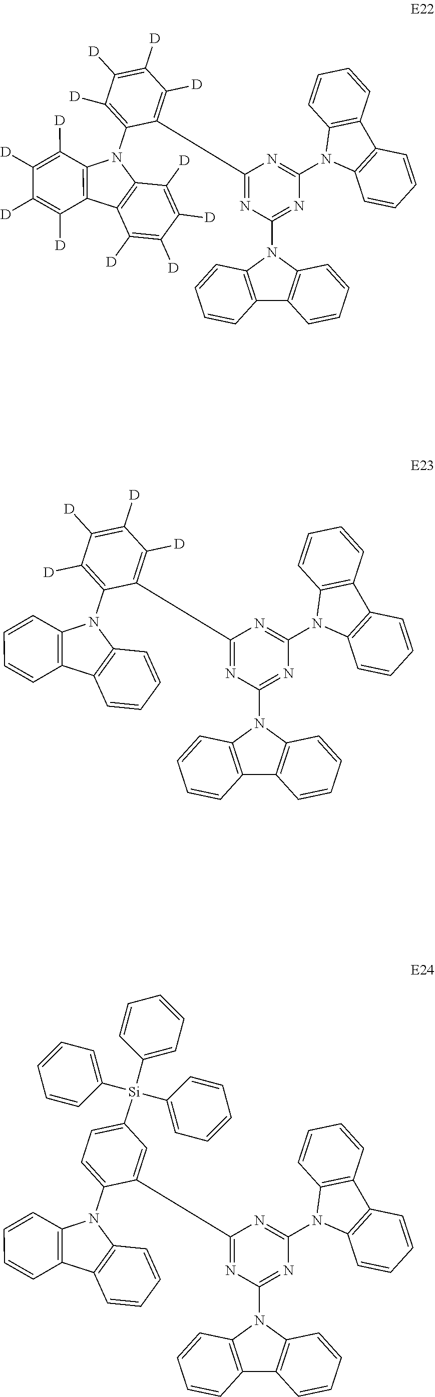 [SMILES: [2H]c1c([2H])c([2H])c(-n2c3c([2H])c([2H])c([2H])c([2H])c3c3c([2H])c([2H])c([2H])c([2H])c32)c(-c2nc(-n3c4ccccc4c4ccccc43)nc(-n3c4ccccc4c4ccccc43)n2)c1[2H] ; [2H]c1c([2H])c([2H])c(-n2c3ccccc3c3ccccc32)c(-c2nc(-n3c4ccccc4c4ccccc43)nc(-n3c4ccccc4c4ccccc43)n2)c1[2H] ; c1ccc([Si](c2ccccc2)(c2ccccc2)c2ccc(-n3c4ccccc4c4ccccc43)c(-c3nc(-n4c5ccccc5c5ccccc54)nc(-n4c5ccccc5c5ccccc54)n3)c2)cc1]

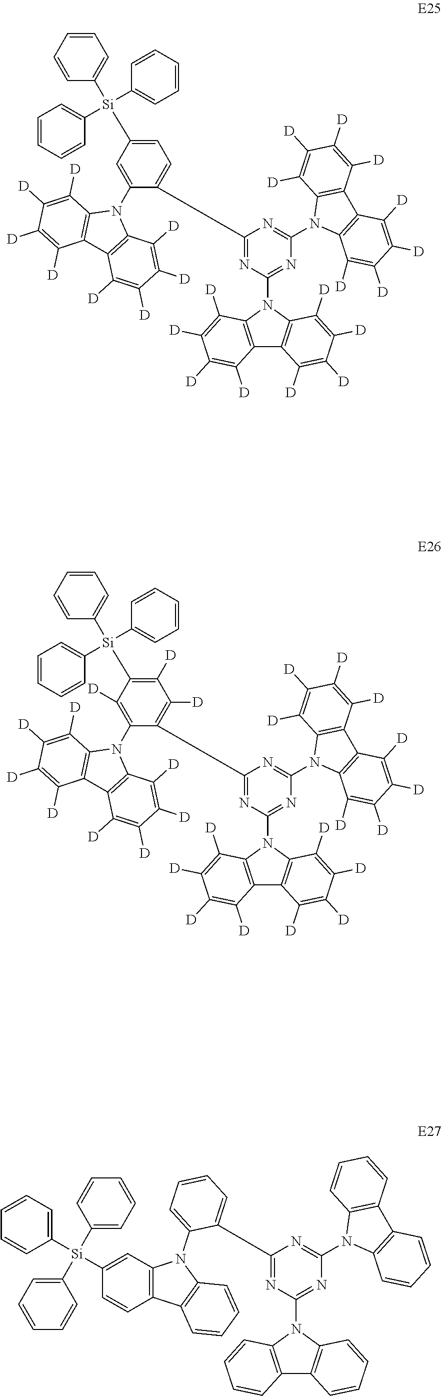

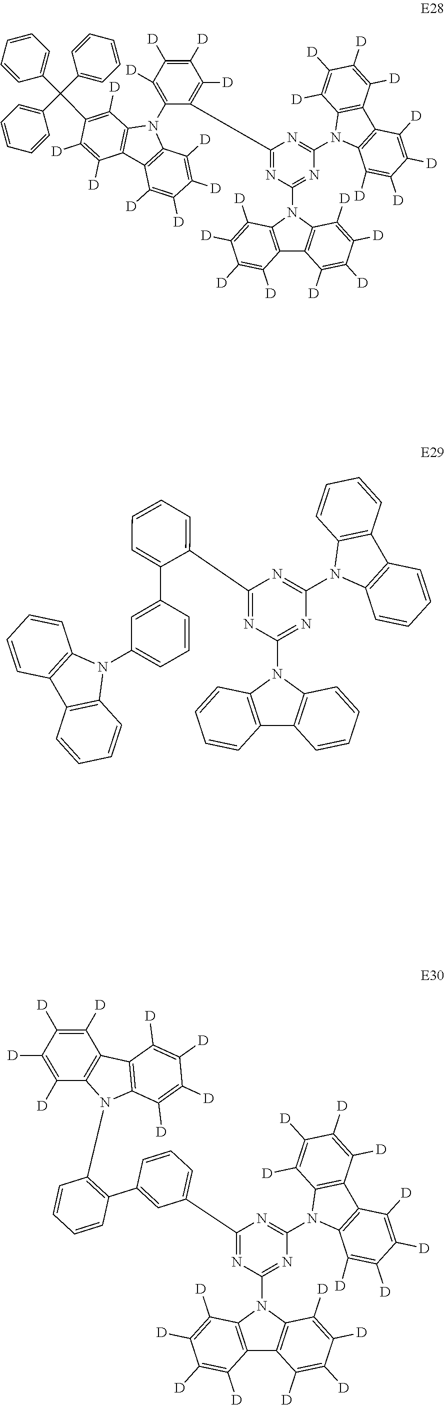

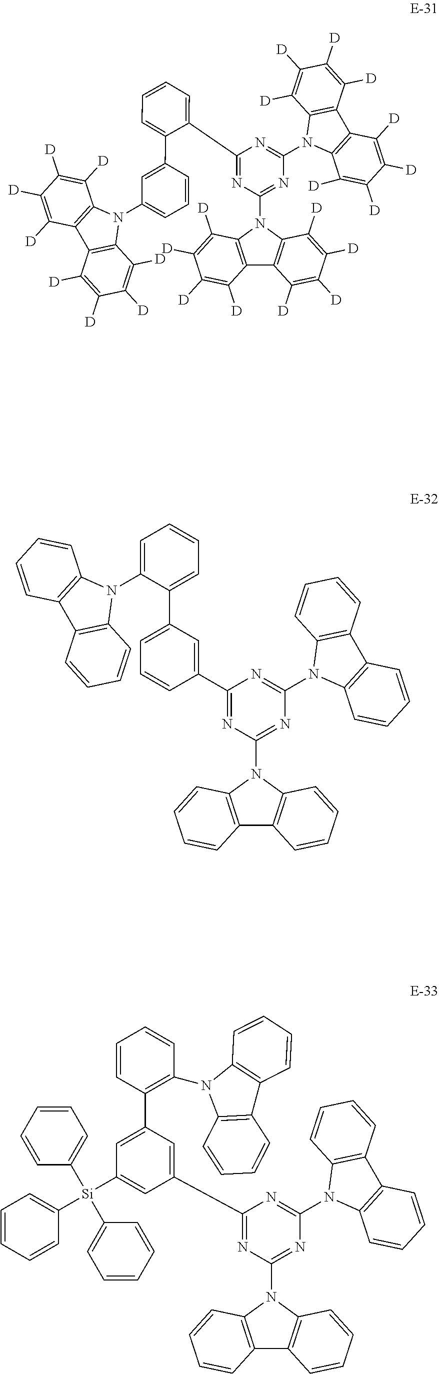 [SMILES: [2H]c1c([2H])c([2H])c2c(c1[2H])c1c([2H])c([2H])c([2H])c([2H])c1n2-c1cccc(-c2ccccc2-c2nc(-n3c4c([2H])c([2H])c([2H])c([2H])c4c4c([2H])c([2H])c([2H])c([2H])c43)nc(-n3c4c([2H])c([2H])c([2H])c([2H])c4c4c([2H])c([2H])c([2H])c([2H])c43)n2)c1 ; c1cc(-c2nc(-n3c4ccccc4c4ccccc43)nc(-n3c4ccccc4c4ccccc43)n2)cc(-c2ccccc2-n2c3ccccc3c3ccccc32)c1 ; c1ccc([Si](c2ccccc2)(c2ccccc2)c2cc(-c3nc(-n4c5ccccc5c5ccccc54)nc(-n4c5ccccc5c5ccccc54)n3)cc(-c3ccccc3-n3c4ccccc4c4ccccc43)c2)cc1]

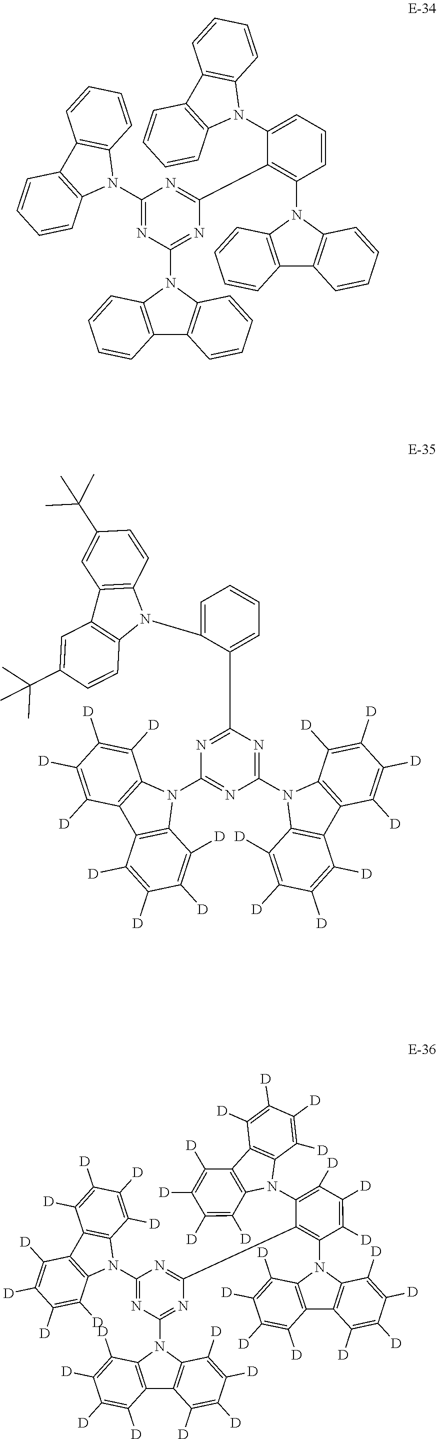

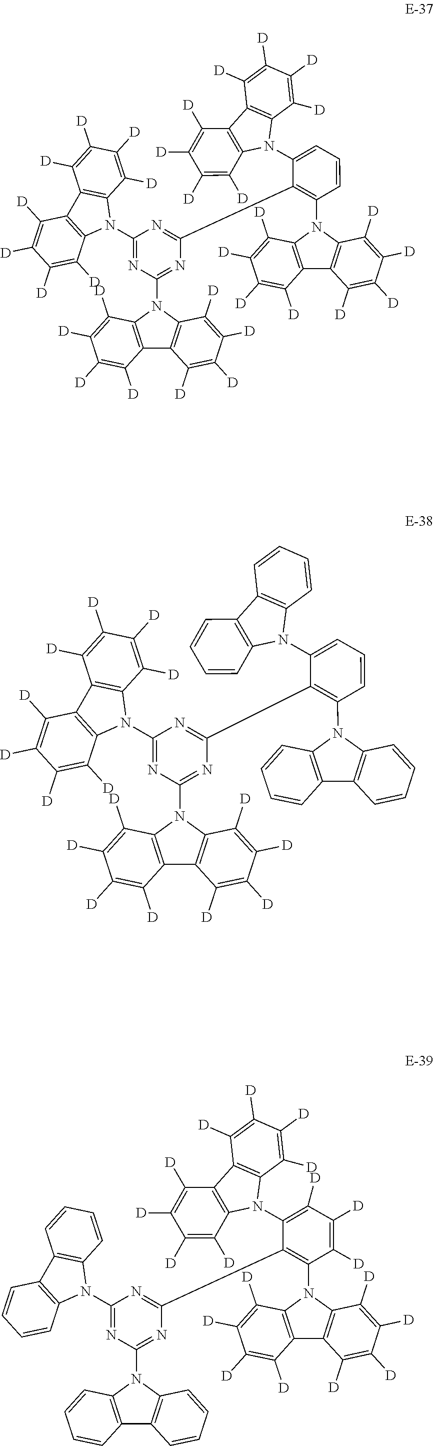

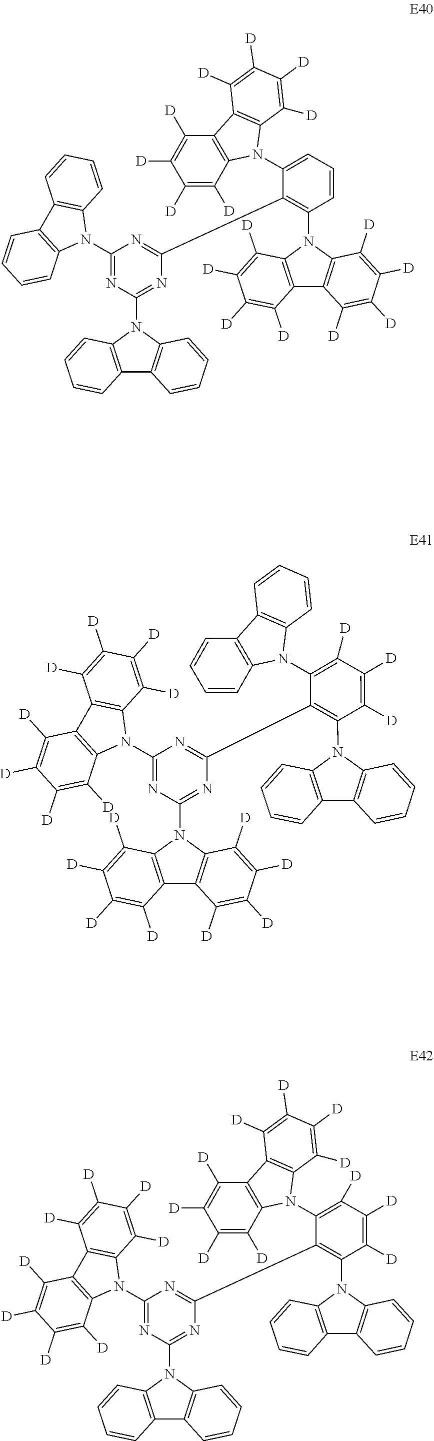

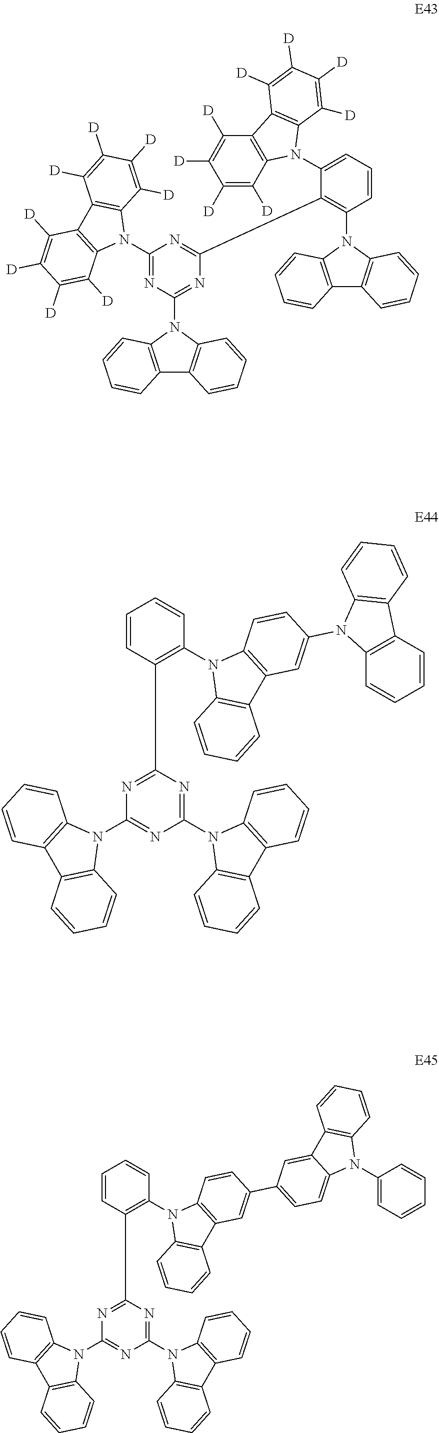 [SMILES: [2H]c1c([2H])c([2H])c2c(c1[2H])c1c([2H])c([2H])c([2H])c([2H])c1n2-c1nc(-c2c(-n3c4ccccc4c4ccccc43)cccc2-n2c3c([2H])c([2H])c([2H])c([2H])c3c3c([2H])c([2H])c([2H])c([2H])c32)nc(-n2c3ccccc3c3ccccc32)n1 ; c1ccc(-n2c3ccccc3c3cc(-n4c5ccccc5c5ccccc54)ccc32)c(-c2nc(-n3c4ccccc4c4ccccc43)nc(-n3c4ccccc4c4ccccc43)n2)c1 ; c1ccc(-n2c3ccccc3c3cc(-c4ccc5c(c4)c4ccccc4n5-c4ccccc4-c4nc(-n5c6ccccc6c6ccccc65)nc(-n5c6ccccc6c6ccccc65)n4)ccc32)cc1]

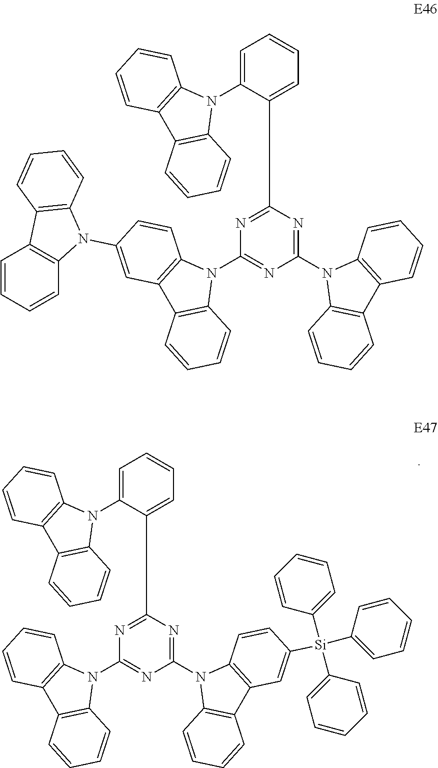 [SMILES: c1ccc(-n2c3ccccc3c3ccccc32)c(-c2nc(-n3c4ccccc4c4ccccc43)nc(-n3c4ccccc4c4cc(-n5c6ccccc6c6ccccc65)ccc43)n2)c1 ; c1ccc([Si](c2ccccc2)(c2ccccc2)c2ccc3c(c2)c2ccccc2n3-c2nc(-c3ccccc3-n3c4ccccc4c4ccccc43)nc(-n3c4ccccc4c4ccccc43)n2)cc1]

The first compound of the above compound is represented by Formula 1, wherein, in Formula 1, i) p+s may be 1 or 2, ii) R₇ may be: hydrogen, deuterium, a C₁-C₆₀ alkyl group, a deuterated C₁-C₆₀ alkyl group; or a phenyl group or a biphenyl group, each unsubstituted or substituted with deuterium, a C₁-C₆₀ alkyl group, a phenyl group, a biphenyl group, or a combination thereof; (that is, R₇ may not include a carbazolyl group), and iii) m2 may be 1 or 2. As a result, the composition including the first compound and the second compound may have a long lifespan, electrical characteristics suitable for blue light emission, and thermal stability, then a light-emitting device of high quality may be manufactured by using the composition.

The composition may further include an emitter. The emitter is a material that may emit light by transition to a ground state by receiving excitons generated from the first compound, the second compound, and/or the sensitizer.

If the composition further includes an emitter, the amount (weight) of the emitter may be in a range of about 0.5 parts by weight to about 30 parts by weight per 100 parts by weight of the composition.

For example, the composition may include a non-emissive material and an emitter, and the first compound and the second compound may be included in the non-emissive material.

As another example, the composition may include a host and an emitter, and the first compound and the second compound may be included in the host.

The emitter may be a compound that may emit phosphorescence or fluorescence.

For example, the emitter may be a phosphorescent compound, a fluorescent compound (e.g., a delayed fluorescent compound, a prompt fluorescent compound, etc.), or a combination thereof.

The emitter may emit blue light.

For example, the emitter may emit blue light having a maximum emission wavelength in a range of about 400 nanometers (nm) to about 490 nm, about 410 nm to about 470 nm, or about 420 nm to about 450 nm.

In one or more embodiments, the emitter may be an organometallic compound, the organometallic compound may include a transition metal and n ligands linked to the transition metal, and n may be an integer from 1 to 4. Thus, the emitter may be a phosphorescent dopant.

In an embodiment, a transition metal of the organometallic compound may be platinum (Pt) or palladium (Pd), n may be 1, and the ligand may be a tetradentate ligand.

The tetradentate ligand may include, for example, a carbene moiety linked to the transition metal.

In one or more embodiments, the transition metal of the organometallic compound may be iridium (Ir) or osmium (Os), n may be 3, and at least one of the n ligands may be a bidentate ligand including —F, a cyano group, or a combination thereof, or a bidentate ligand including a carbene moiety linked to the transition metal. For example, the bidentate ligand may further include an imidazole group or a triazole group.

In one or more embodiments, the organometallic compound may be an organometallic compound represented by Formula 3 and/or an organometallic compound represented by Formula 5. Formulae 3 and 5 are each as described herein.

In an embodiment, the emitter may be a delayed fluorescence material. For example, the emitter may be a thermally activated delayed fluorescence material. In an embodiment, the emitter may be a multiple resonance thermally activated delayed fluorescence material.

The multiple resonance thermally activated delayed fluorescence material may be a polycyclic compound i) not including a transition metal and ii) including a core in which two or more C₃-C₆₀ cyclic groups are condensed to each other. Here, at least two C₃-C₆₀ cyclic groups of the core may be condensed to each other while sharing boron (B) or nitrogen (N).

In an embodiment, the emitter may be a polycyclic compound represented by Formula 4. Formula 4 will be described in detail below.

In one or more embodiments, the composition may further include a sensitizer.

An amount (weight) of the sensitizer may be about 0.01 parts by weight to about 10 parts by weight per 100 parts by weight of the composition.

For example, the sensitizer may be an organometallic compound including a transition metal and a tetradentate ligand linked to the transition metal, the transition metal may be platinum (Pt) or palladium (Pd), and the tetradentate ligand may include a carbene moiety linked to the transition metal.

In an embodiment, the composition may further include an emitter and a sensitizer, in addition to the first compound and the second compound described herein. Excitons generated in the first compound and the second compound may be effectively transferred to the emitter through the sensitizer.

In an embodiment, the emitter may be a delayed fluorescence material as described herein, and the sensitizer may be an organometallic compound as described herein. For example, the emitter may be a polycyclic compound represented by Formula 4, and the sensitizer may be an organometallic compound represented by Formula 3.

### Description of Formula 3

The emitter and the sensitizer as described herein may each be an organometallic compound represented by Formula 3:

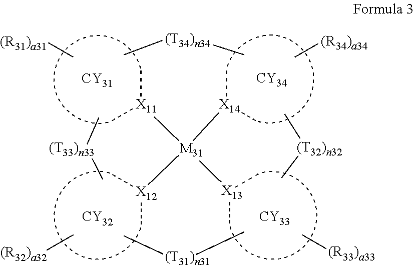

wherein, in Formula 3,
M₃₁ may be a transition metal,
X₁₁ to X₁₄ may each independently be C or N,
two bonds of a bond between X₁₁ and M₃₁, a bond between X₁₂ and M₃₁, a bond between X₁₃ and M₃₁, and a bond between X₁₄ and M₃₁ may each be a coordinate bond, and the other two bonds may each be a covalent bond, ring CY₃₁ to ring CY₃₄ may each independently be a C₅-C₃₀ carbocyclic group or a C₁-C₃₀ heterocyclic group,
T₃₁ may be a single bond, a double bond, *—N(R₃₅ₐ)—*′, *—B(R₃₅ₐ)—*′, *—P(R₃₅ₐ)—*′, *—C(R₃₅ₐ)(R_{35b})—*′, *—Si(R₃₅ₐ)(R_{35b})—*′, *—Ge(R₃₅ₐ)(R_{35b})—*′, *—S—*′, *—Se—*′, *—O—*′, *—C(═O)—*′, *—S(═O)—*′, *—S(═O)₂—*′, *—C(R₃₅ₐ)═*′, *═C(R₃₅ₐ)—*′, *—C(R₃₅ₐ)═C(R_{35b})—*′, *—C(═S)—*′, *—C* a C₅-C₃₀ carbocyclic group unsubstituted or substituted with at least one R₁₀ₐ, or a C₁-C₃₀ heterocyclic group unsubstituted or substituted with at least one R₁₀ₐ,
T₃₂ may be a single bond, a double bond, *—N(R₃₆ₐ)—*′, *—B(R₃₆ₐ)—*′, *—P(R₃₆ₐ)—*′, *—C(R₃₆ₐ)(R_{36b})—*′, *—Si(R₃₆ₐ)(R_{36b})—*′, *—Ge(R₃₆ₐ)(R_{36b})—*′, *—S—*′, *—Se—*′, *—O—*′, *—C(═O)—*′, *—S(═O)—*′, *—S(═O)₂—*′, *—C(R₃₆ₐ)═*′, *═C(R₃₆ₐ)—*′, *—C(R₃₆ₐ)═C(R_{36b})—*′, *—C(═S)—*′, *—CGC-*′, a C₅-C₃₀ carbocyclic group unsubstituted or substituted with at least one R₁₀ₐ, or a C₁-C₃₀ heterocyclic group unsubstituted or substituted with at least one R₁₀ₐ,
T₃₃ may be a single bond, a double bond, *—N(R₃₇ₐ)—*′, *—B(R₃₇ₐ)—*′, *—P(R₃₇ₐ)—*′, *—C(R₃₇ₐ)(R_{37b})—*′, *—Si(R₃₇ₐ)(R_{37b})—*′, *—Ge(R₃₇ₐ)(R_{37b})—*′, *—S—*′, *—Se—*′, *—O—*′, *—C(═O)—*′, *—S(═O)—*′, *—S(═O)₂—*′, *—C(R₃₇ₐ)═*′, *═C(R₃₇ₐ)—*′, *—C(R₃₇ₐ)═C(R_{37b})—*′, *—C(═S)—*′, *—C≡C—*′ a C₅-C₆₀ carbocyclic group unsubstituted or substituted with at least one R₁₀ₐ, or a C₁-C₃₀ heterocyclic group unsubstituted or substituted with at least one R₁₀ₐ,
T₃₄ may be a single bond, a double bond, *—N(R₃₈ₐ)—*′, *—B(R₃₈ₐ)—*′, *—P(R₃₈ₐ)—*′, *—C(R₃₈ₐ)(R_{38b})—*′, *—Si(R₃₈ₐ)(R_{38b})—*′, *—Ge(R₃₈ₐ)(R_{38b})—*′, *—S—*′, *—Se—*′, *—O—*′, *—C(═O)—*′, *—S(═O)—*′, *—S(═O)₂—*′, *—C(R₃₈ₐ)═*′, *═C(R₃₈ₐ)—*′, *—C(R₃₈ₐ)═C(R_{38b})—*′, *—C(═S)—*′, *—C* a C₅-C₃₀ carbocyclic group unsubstituted or substituted with at least one R₁₀ₐ, or a C₁-C₃₀ heterocyclic group unsubstituted or substituted with at least one R₁₀ₐ,
n31 to n34 may each independently be an integer from 0 to 5, and three or more of n31 to n34 may each independently be an integer from 1 to 5, when n31 is 0, T₃₁ may not be present, when n32 is 0, T₃₂ may not be present, when n33 is 0, T₃₃ may not be present, and when n34 is 0, T₃₄ may not be present, when n31 is 2 or more, two or more of T₃₁(s) may be identical to or different from each other, when n32 is 2 or more, two or more of T₃₂(s) may be identical to or different from each other, when n33 is 2 or more, two or more of T₃₃(s) may be identical to or different from each other, and when n34 is 2 or more, two or more of T₃₄(s) may be identical to or different from each other, R₃₁ to R₃₄, R₃₅ₐ, R_{35b}, R₃₆ₐ, R_{36b}, R₃₇ₐ, R_{37b}, R₃₈ₐ, and R_{38b} may each independently be hydrogen, deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ arylalkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ heteroarylalkyl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉),
a31 to a34 may each independently be an integer from 0 to 20, two or more of R₃₁(s) in the number of a31 may optionally be bonded to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
two or more of R₃₂(s) in the number of a32 may optionally be bonded to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
two or more of R₃₃(s) in the number of a33 may optionally be bonded to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
two or more of R₃₄(s) in the number of a34 may optionally be bonded to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
two or more of R₃₁ to R₃₄, R₃₅ₐ, R_{35b}, R₃₆ₐ, R_{36b}, R₃₇ₐ, R_{37b}, R₃₈ₐ, and R_{38b} may optionally be bonded to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
R₁₀ₐ is as described for R₃₁,
* and *′ each indicate a binding site to a neighboring atom,
a substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₇-C₆₀ arylalkylgroup, the substituted C₆-C₆₀ aryloxy group, the substituted C₆-C₆₀ arylthio group, the substituted C₁-C₆₀ heteroaryl group, the substituted C₂-C₆₀ heteroarylalkyl group, the substituted monovalent non-aromatic condensed polycyclic group, and the substituted monovalent non-aromatic condensed heteropolycyclic group may be:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or a combination thereof;
a C₃-C₁₀ cycloalkyl group, a C₁-C₁ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁ cycloalkyl group, a C₁-C₁ heterocycloalkyl group, a C₃-C₁₀ cycloalkenyl group, a C₂-C₁ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉), or a combination thereof;
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉); or
a combination thereof,
wherein Q₁ to Q₉, Q₁₁ to Q₁₉, Q₂₁ to Q₂₉, and Q₃₁ to Q₃₉ are each independently hydrogen; deuterium; —F; —Cl; —Br; —I; a hydroxyl group; a cyano group; a nitro group; an amidino group; a hydrazine group; a hydrazone group; a carboxylic acid group or a salt thereof; a sulfonic acid group or a salt thereof; a phosphoric acid group or a salt thereof; or a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, unsubstituted or substituted with deuterium, a C₁-C₆₀ alkyl group, a C₆-C₆₀ aryl group, or a combination thereof.

In an embodiment, M₃₁ in Formula 3 may be Pt, Pd, or Au.

In one or more embodiments, M₃₁ in Formula 3 may be Pt or Pd.

In one or more embodiments, a bond between X₁₁ and M₃₁ in Formula 3 may be a coordinate bond.

In one or more embodiments, in Formula 3, X₁₁ may be C, and a bond between X₁₁ and M₃₁ may be a coordinate bond. That is, X₁₁ in Formula 3 may be C in a carbene moiety.

In one or more embodiments, ring CY₃₁ to ring CY₃₄ in Formula 3 may each independently be i) a first ring, ii) a second ring, iii) a condensed ring in which two or more first rings are condensed with each other, iv) a condensed ring in which two or more second rings are condensed with each other, or v) a condensed ring in which at least one first ring is condensed with at least one second ring,
the first ring may be a cyclopentane group, a cyclopentadiene group, a furan group, a thiophene group, a pyrrole group, a silole group, an oxazole group, an isoxazole group, an oxadiazole group, an isoxadiazole group, an oxatriazole group, an isoxatriazole group, a thiazole group, an isothiazole group, a thiadiazole group, an isothiadiazole group, a thiatriazole group, an isothiatriazole group, a pyrazole group, an imidazole group, a triazole group, a tetrazole group, an azasilole group, a diazasilole group, or a triazasilole group, and
the second ring may be an adamantane group, a norbornane group, a norbornene group, a cyclohexane group, a cyclohexene group, a benzene group, a pyridine group, a pyrimidine group, a pyrazine group, a pyridazine group, a triazine group, an oxazine group, a thiazine group, a dihydropyrazine group, a dihydropyridine group, or a dihydroazasiline group.

In an embodiment, R₃₁ to R₃₄, R₃₅ₐ, R_{35b}, R₃₆ₐ, R_{36b}, R₃₇ₐ, R_{37b}, R₃₈ₐ, and R_{38b} may each independently be:
hydrogen, deuterium, —F, —Cl, —Br, —I, —CD₃, —CD₂H, —CDH₂, —CF₃, —CF₂H, —CFH₂, a cyano group (CN), a nitro group, an amino group, a C₁-C₂₀ alkyl group, or a C₁-C₂₀ alkoxy group;
a C₁-C₂₀ alkyl group or a C₁-C₂₀ alkoxy group, each substituted with at least one of deuterium, —F, —Cl, —Br, —I, —CD₃, —CD₂H, —CDH₂, —CF₃, —CF₂H, —CFH₂, a cyano group (CN), a nitro group, an amino group, and a phenyl group; or
a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, or an anthracenyl group, each unsubstituted or substituted with at least one of deuterium, —F, —Cl, —Br, —I, —CD₃, —CD₂H, —CDH₂, —CF₃, —CF₂H, —CFH₂, a cyano group (CN), a nitro group, an amino group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, and an anthracenyl group.

In an embodiment, the organometallic compound represented by Formula 3 may be an organometallic compound represented by Formula 3-1 or an organometallic compound represented by Formula 3-2:

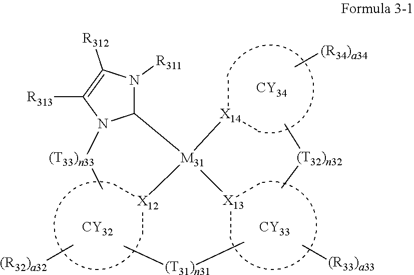

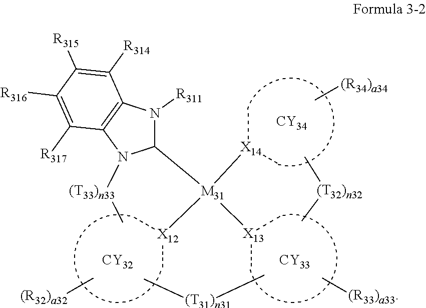

A bond between a carbon of an imidazole group and M₃₁ in Formula 3-1 may be a coordinate bond. That is, the imidazole group of Formula 3-1 may include a carbene moiety linked to M₃₁.

A bond between a carbon of a benzimidazole group and M₃₁ in Formula 3-2 may be a coordinate bond. That is, the benzimidazole group of Formula 3-2 may include a carbene moiety linked to M₃₁.

In Formulae 3-1 and 3-2,
M₃₁, CY₃₂, CY₃₃, CY₃₄, X₁₂, X₁₃, X₁₄, T₃₁, T₃₂, T₃₃, n31, n32, n33, R₃₂, R₃₃, R₃₄, a32, a33, and a34 may respectively be understood by referring to the descriptions of M₃₁, CY₃₂, CY₃₃, CY₃₄, X₁₂, X₁₃, X₁₄, T₃₁, T₃₂, T₃₃, n31, n32, n33, R₃₂, R₃₃, R₃₄, a32, a33, and a34 provided herein, and
R₃₁₁ to R₃₁₇ may each be understood by referring to the description of R₃₁ provided herein.

In an embodiment, in Formulae 3-1 and 3-2, R₃₁₁ to R₃₁₇ may each independently be:
hydrogen, deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid group or a salt thereof, a sulfonic acid group or a salt thereof, or a phosphoric acid group or a salt thereof;
a C₁-C₂₀ alkyl group or a C₁-C₂₀ alkoxy group, each unsubstituted or substituted with deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, or a combination thereof;
a C₃-C₁ cycloalkyl group, a C₁-C₁ heterocycloalkyl group, a C₆-C₆₀ aryl group, a C₇-C₆₀ arylalkyl group, C₆-C₆₀ aryloxy group, a C₆-C₆₀ arylthio group, a C₁-C₆₀ heteroaryl group, a C₂-C₆₀ heteroarylalkyl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, —SFs, a hydroxyl group, a cyano group, a nitro group, an amidino group, a hydrazine group, a hydrazone group, a C₁-C₂₀ alkyl group, a C₁-C₂₀ alkoxy group, or a combination thereof; or
—N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉).

For example, in Formulae 3-1 and 3-2,
at least one of R₃₁₁ to R₃₁₇ may be
a C₁-C₂₀ alkyl group, a C₆-C₆₀ aryl group, or a C₇-C₆₀ arylalkyl group, each unsubstituted or substituted with at least one of a methyl group, an ethyl group, an n-propyl group, an iso-propyl group, an n-butyl group, an isobutyl group, a sec-butyl group, a tert-butyl group, a phenyl group, a cumyl group, or a combination thereof.

### Description of Formula 5

The emitter as used herein may be an organometallic compound represented by Formula 5:

M₅₁(L₅₁)ₙ₅₁(L₅₂)ₙ₅₂ Formula 5

wherein, in Formula 5, M₅₁ may be a transition metal.

In some embodiments, M₅₁ may be a first-row transition metal, a second-row transition metal, or a third-row transition metal.

In some embodiments, M₅₁ may be iridium (Ir), platinum (Pt), osmium (Os), titanium (T₁), zirconium (Zr), hafnium (Hf), europium (Eu), terbium (Tb), thulium (Tm), or rhodium (Rh).

In some embodiments, M₅₁ may be Ir, Pt, Os, or Rh.

In one or more embodiments, M₅₁ may be Ir or Os.

In Formula 5, L₅₁ may be a ligand represented by Formula 5A, and L₅₂ may be a ligand represented by Formula 5B:

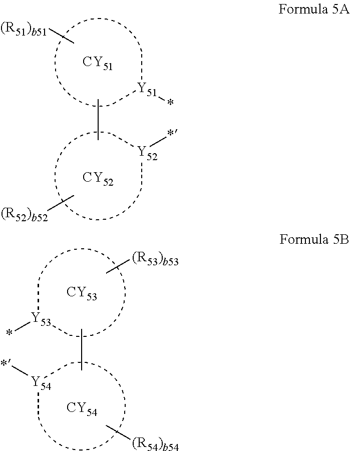

wherein Formulae 5A and 5B are each as described herein.

In Formula 5, n51 may be 1, 2, or 3, wherein, when n51 is 2 or more, two or more of L₅₁ (s) may be identical to or different from each other.

In Formula 5, n52 may be 0, 1, or 2, wherein, when n52 is 2, two L₅₂(s) may be identical to or different from each other.

The sum of n51 and n52 in Formula 5 may be 2 or 3. For example, the sum of n51 and n52 may be 3.

In an embodiment, in Formula 5, i) M may be Ir, and n51+n52=3; or ii) M may be Pt, and n51+n52=2.

In one or more embodiments, in Formula 5, M may be Ir, and i) n51 may be 1, and n52 may be 2, or ii) n51 may be 2, and n52 may be 1.

L₅₁ and L₅₂ in Formula 5 may be different from each other.

Y₅₁ to Y₅₄ in Formulae 5A to 5B may each independently be C or N. For example, Y₅₁ and Y₅₃ may each be N, and Y₅₂ and Y₅₄ may each be C.

Ring CY₅₁ to ring CY₅₄ in Formulae 5A and 5B may each independently be a C₅-C₃₀ carbocyclic group or a C₁-C₃₀ heterocyclic group.

For example, ring CY₅₁ to ring CY₅₄ in Formulae 5A and 5B may each independently include i) a third ring, ii) a fourth ring, iii) a condensed ring in which two or more third rings are condensed with each other, iv) a condensed ring in which two or more fourth rings are condensed with each other, or v) a condensed ring in which at least one third ring is condensed with at least one fourth ring, the third ring may be a cyclopentane group, a cyclopentene group, a furan group, a thiophene group, a pyrrole group, a silole group, a borole group, a phosphole group, a germole group, a selenophene group, an oxazole group, an oxadiazole group, an oxatriazole group, a thiazole group, a thiadiazole group, a thiatriazole group, a pyrazole group, an imidazole group, a triazole group, a tetrazole group, or an azasilole group, and the fourth ring may be an adamantane group, a norbornane group, a norbornene group, a cyclohexane group, a cyclohexene group, a benzene group, a pyridine group, a pyrimidine group, a pyrazine group, a pyridazine group, or a triazine group.

In some embodiments, in Formulae 5A and 5B, ring CY₅₁ to ring CY₅₄ may each independently be a cyclopentane group, a cyclohexane group, a cyclohexene group, a benzene group, a naphthalene group, an anthracene group, a phenanthrene group, a triphenylene group, a pyrene group, a chrysene group, a 1,2,3,4-tetrahydronaphthalene group, a cyclopentadiene group, a pyrrole group, a furan group, a thiophene group, a silole group, a borole group, a phosphole group, a germole group, a selenophene group, an indene group, an indole group, a benzofuran group, a benzothiophene group, a benzosilole group, a benzoborole group, a benzophosphole group, a benzogermole group, a benzoselenophene group, a fluorene group, a carbazole group, a dibenzofuran group, a dibenzothiophene group, a dibenzosilole group, a dibenzoborole group, a dibenzophosphole group, a dibenzogermole group, a dibenzoselenophene group, a benzofluorene group, a benzocarbazole group, a naphthobenzofuran group, a naphthobenzothiophene group, a naphthobenzosilole group, a naphthobenzoborole group, a naphthobenzophosphole group, a naphthobenzogermole group, a naphthobenzoselenophene group, a dibenzofluorene group, a dibenzocarbazole group, a dinaphthofuran group, a dinaphthothiophene group, a dinaphthosilole group, a dinaphthoborole group, a dinaphthophosphole group, a dinaphthogermole group, a dinaphthoselenophene group, an indenophenanthrene group, an indolophenanthrene group, a phenanthrobenzofuran group, a phenanthrobenzothiophene group, a phenanthrobenzosilole group, a phenanthrobenzoborole group, a phenanthrobenzophosphole group, a phenanthrobenzogermole group, a phenanthrobenzoselenophene group, a dibenzothiophene 5-oxide group, a 9H-fluorene-9-one group, a dibenzothiophene 5,5-dioxide group, an azaindene group, an azaindole group, an azabenzofuran group, an azabenzothiophene group, an azabenzosilole group, an azabenzoborole group, an azabenzophosphole group, an azabenzogermole group, an azabenzoselenophene group, an azafluorene group, an azacarbazole group, an azadibenzofuran group, an azadibenzothiophene group, an azadibenzosilole group, an azadibenzoborole group, an azadibenzophosphole group, an azadibenzogermole group, an azadibenzoselenophene group, an azabenzofluorene group, an azabenzocarbazole group, an azanaphthobenzofuran group, an azanaphthobenzothiophene group, an azanaphthobenzosilole group, an azanaphthobenzoborole group, an azanaphthobenzophosphole group, an azanaphthobenzogermole group, an azanaphthobenzoselenophene group, an azadibenzofluorene group, an azadibenzocarbazole group, an azadinaphthofuran group, an azadinaphthothiophene group, an azadinaphthosilole group, an azadinaphthoborole group, an azadinaphthophosphole group, an azadinaphthogermole group, an azadinaphthoselenophene group, an azaindenophenanthrene group, an azaindolophenanthrene group, an azaphenanthrobenzofuran group, an azaphenanthrobenzothiophene group, an azaphenanthrobenzosilole group, an azaphenanthrobenzoborole group, an azaphenanthrobenzophosphole group, an azaphenanthrobenzogermole group, an azaphenanthrobenzoselenophene group, an azadibenzothiophene 5-oxide group, an aza9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a benzoquinoline group, a benzoisoquinoline group, a benzoquinoxaline group, a benzoquinazoline group, a phenanthroline group, a phenanthridine group, a pyrrole group, a pyrazole group, an imidazole group, a triazole group, an oxazole group, an isooxazole group, a thiazole group, an isothiazole group, an oxadiazole group, a thiadiazole group, an azasilole group, an azaborole group, an azaphosphole group, an azagermole group, an azaselenophene group, a benzopyrrole group, a benzopyrazole group, a benzimidazole group, a benzoxazole group, a benzisoxazole group, a benzothiazole group, a benzoisothiazolyl group, a benzoxadiazole group, a benzothiadiazole group, a pyridinopyrrole group, a pyridinopyrazole group, a pyridinoimidazole group, a pyridinooxazole group, a pyridinoisoxazole group, a pyridinothiazole group, a pyridinoisothiazole group, a pyridinooxadiazole group, a pyridinothiadiazole group, a pyrimidinopyrrole group, a pyrimidinopyrazole group, a pyrimidinoimidazole group, a pyrimidinooxazole group, a pyrimidinoisoxazole group, a pyrimidinothiazole group, a pyrimidinoisothiazole group, a pyrimidinooxadiazole group, a pyrimidinothiadiazole group, a 5,6,7,8-tetrahydroisoquinoline group, a 5,6,7,8-tetrahydroquinoline group, an adamantane group, a norbornane group, a norbornene group, a benzene group condensed with a cyclohexane group, a benzene group condensed with a norbornane group, a pyridine group condensed with a cyclohexane group, or a pyridine group condensed with a norbornane group.

For example, ring CY₅₁ and ring CY₅₃ in Formulae 5A and 5B may be different from each other.

In one or more embodiments, ring CY₅₂ and ring CY₅₄ in Formulae 5A and 5B may be different from each other.

In one or more embodiments, ring CY₅₁ to ring CY₅₄ in Formulae 5A and 5B may be different from each other.

R₅₁ to R₅₄ in Formulae 5A and 5B may each independently be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₅₁)(Q₅₂), —Si(Q₅₃)(Q₅₄)(Qss), —Ge(Q₅₃)(Q₅₄)(Q₅₅), —B(Q₅₆)(Q₅₇), —P(═O)(Q₅₈)(Q₅₉), or —P(Q₅₈)(Q₅₉). 051 to Q₅₉ are each as described herein.

In an embodiment, R₅₁ to R₅₄ in Formulae 5A and 5B may each independently be:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, a C₁-C₂₀ alkoxy group, or a C₁-C₂₀ alkylthio group;
a C₁-C₂₀ alkyl group, a C₁-C₂₀ alkoxy group, or a C₁-C₂₀ alkylthio group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a pyridinyl group, a pyrimidinyl group, or a combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group or azadibenzothiophenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a (phenyl)C₁-C₁₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, or a combination thereof; or
—N(Q₅₁)(Q₅₂), —Ge(Q₅₃)(Q₅₄)(Q₅₅), —B(Q₅₆)(Q₅₇), —P(═O)(Q₅₈)(Q₅₉), or —P(Q₅₈)(Q₅₉),
wherein 051 to Q₅₉ may each independently be: —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, a C₁-C₁ alkyl group, a phenyl group, or a combination thereof.

In one or more embodiments, R₅₁ to R₅₄ may each independently be:
hydrogen, deuterium, —F, or a cyano group;
a C₁-C₂₀ alkyl group unsubstituted or substituted with deuterium, a cyano group, a C₃-C₁₀ cycloalkyl group, a deuterated C₃-C₁₀ cycloalkyl group, a fluorinated C₃-C₁₀ cycloalkyl group, a (C₁-C₂₀ alkyl)C₃-C₁₀ cycloalkyl group, a C₁-C₁ heterocycloalkyl group, a deuterated C₁-C₁ heterocycloalkyl group, a fluorinated C₁-C₁ heterocycloalkyl group, a (C₁-C₂₀ alkyl)C₁-C₁ heterocycloalkyl group, a phenyl group, a deuterated a phenyl group, a fluorinated a phenyl group, a (C₁-C₂₀ alkyl)phenyl group, a biphenyl group, a deuterated a biphenyl group, a fluorinated a biphenyl group, a (C₁-C₂₀ alkyl)biphenyl group, a dibenzofuranyl group, a deuterated a dibenzofuranyl group, a fluorinated a dibenzofuranyl group, a (C₁-C₂₀ alkyl)dibenzofuranyl group, a dibenzothiophenyl group, a deuterated a dibenzothiophenyl group, a fluorinated a dibenzothiophenyl group, a (C₁-C₂₀ alkyl)dibenzothiophenyl group, or a combination thereof;
a C₃-C₁ cycloalkyl group, a C₁-C₁ heterocycloalkyl group, a phenyl group, or a biphenyl group, each unsubstituted or substituted with deuterium, a cyano group, a C₁-C₂₀ alkyl group, a deuterated C₁-C₂₀ alkyl group, fluorinated C₁-C₂₀ alkyl group, a C₁-C₂₀ alkoxy group, a deuterated C₁-C₂₀ alkoxy group, a fluorinated C₁-C₂₀ alkoxy group, a C₃-C₁ cycloalkyl group, a deuterated C₃-C₁ cycloalkyl group, a fluorinated C₃-C₁ cycloalkyl group, a (C₁-C₂₀ alkyl)C₃-C₁₀ cycloalkyl group, a C₁-C₁ heterocycloalkyl group, a deuterated C₁-C₁ heterocycloalkyl group, a fluorinated C₁-C₁ heterocycloalkyl group, a (C₁-C₂a alkyl)C₁-C₁ heterocycloalkyl group, a phenyl group, a deuterated phenyl group, a fluorinated a phenyl group, a (C₁-C₂₀ alkyl)phenyl group, a biphenyl group, a deuterated biphenyl group, a fluorinated a biphenyl group, a (C₁-C₂₀ alkyl)biphenyl group, a dibenzofuranyl group, a deuterated dibenzofuranyl group, a fluorinated a dibenzofuranyl group, a (C₁-C₂₀ alkyl)dibenzofuranyl group, a dibenzothiophenyl group, a deuterated dibenzothiophenyl group, a fluorinated a dibenzothiophenyl group, a (C₁-C₂₀ alkyl)dibenzothiophenyl group, or a combination thereof; or
—Si(Q₅₃)(Q₅₄)(Q₅₅) or —Ge(Q₅₃)(Q₅₄)(Q₅₅).

The b51 to b54 in Formulae 5A and 5B indicate the numbers of R₅₁ to R₅₄, respectively, and may each independently be an integer from 0 to 20. When b51 is 2 or more, two or more of R₅₁(s) may be identical to or different from each other, when b52 is 2 or more, two or more of R₅₂(s) may be identical to or different from each other, when b53 is 2 or more, two or more of R₅₃(s) may be identical to or different from each other, and when b54 is 2 or more, two or more of R₅₄(s) may be identical to or different from each other. For example, b51 to b54 may each independently be an integer from 0 to 8.

In an embodiment, Y₅₂ in Formula 5A may be C, a bond between Y₅₂ and M₅₁ may be a covalent bond, and at least one of R₅₂ in the number of b52 may be a cyano group or —F. When b52 is at least two, the at least two of R₅₂ may be a cyano group, —F, or a combination thereof.

In one or more embodiments, Y₅₁ in Formula 5A may be N, a bond between Y₅₁ and M₅₁ may be a coordinate bond, CY₅₁ may be an imidazole group, a triazole group, a benzimidazole group, or a triazolopyridine group, and at least one of R₅₂ in the number of b52 may be a cyano group or —F. When b52 is at least two, the at least two of R₅₂ may be a cyano group, —F, or a combination thereof.

In one or more embodiments, Y₅₁ in Formula 5A may be C, and a bond between Y₅₁ and M₅₁ may be a coordinate bond.

In one or more embodiments, Y₅₁ in Formula 5A may be C, a bond between Y₅₁ and M₅₁ may be a coordinate bond, and CY₅₁ may be a benzimidazole group or an imidazopyrazine group.

Examples of an organometallic compound represented by Formula 3 and an organometallic compound represented by Formula 5.

For example, the organometallic compound represented by Formula 3 and the organometallic compound represented by Formula 5 may be one of Compounds P1 to P52:

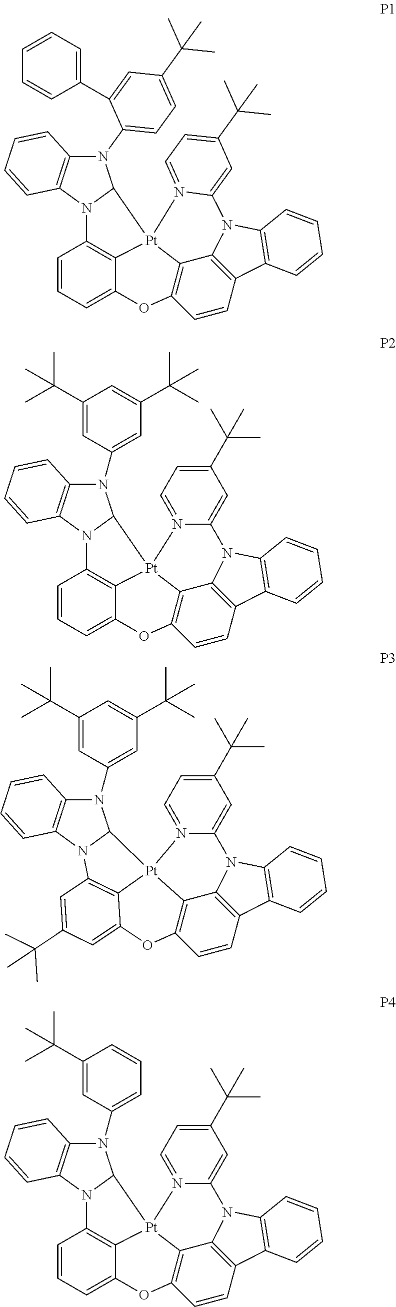

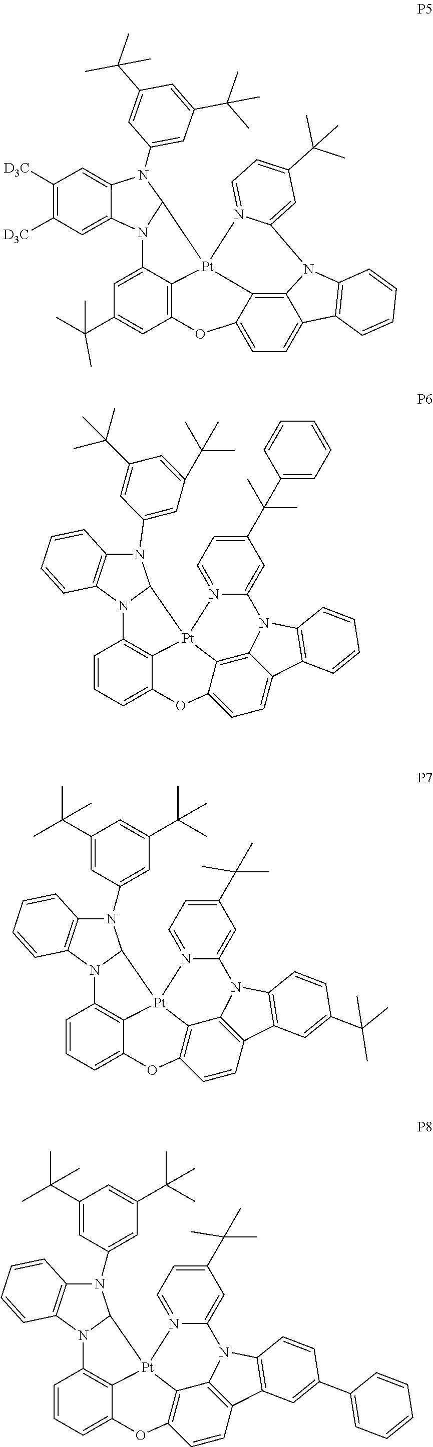

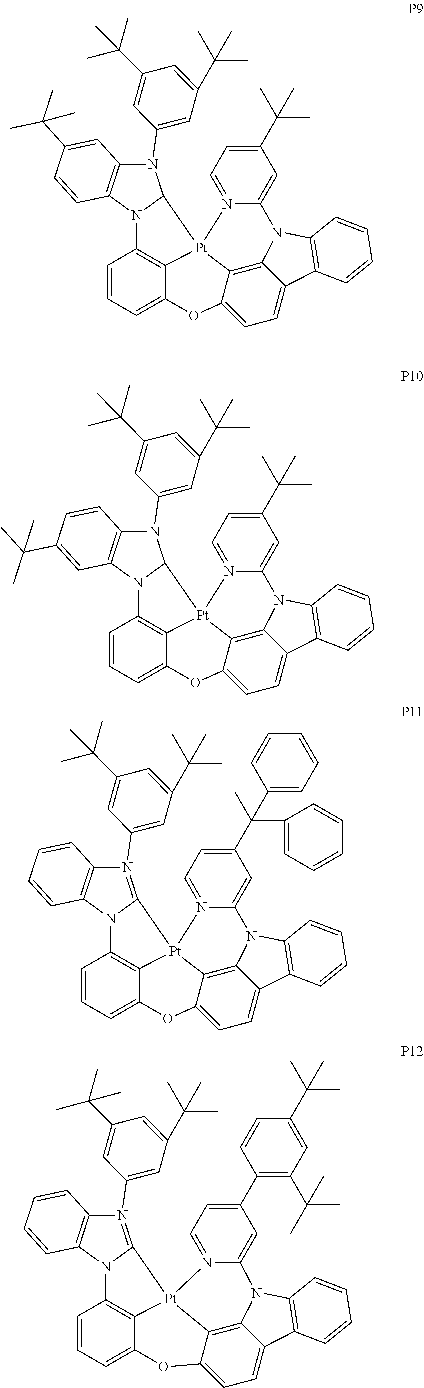

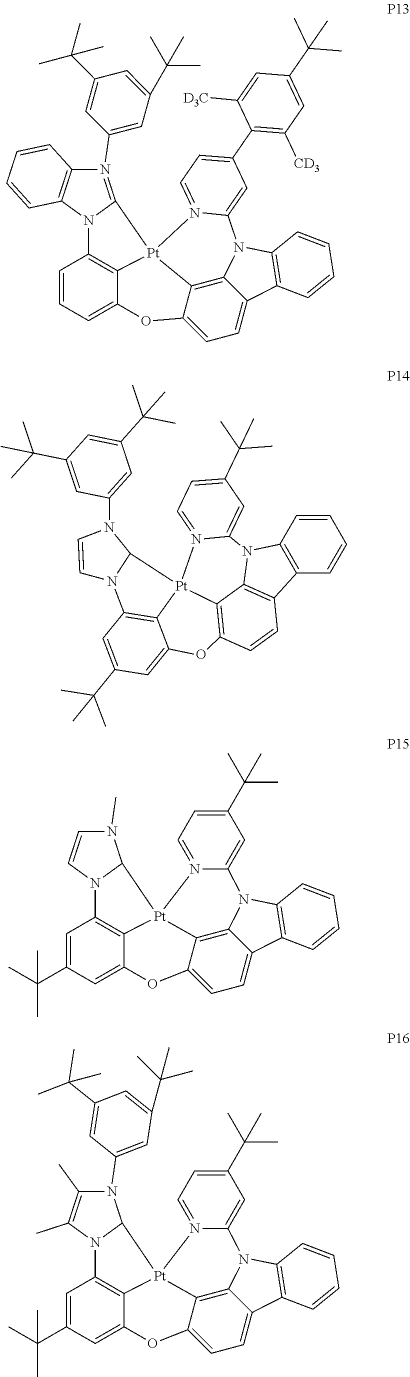

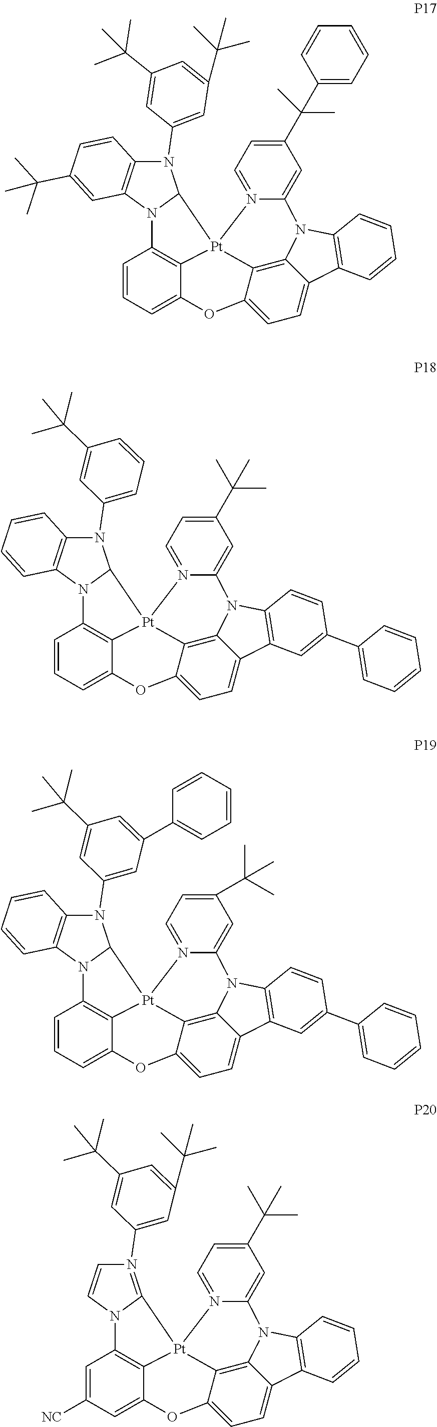

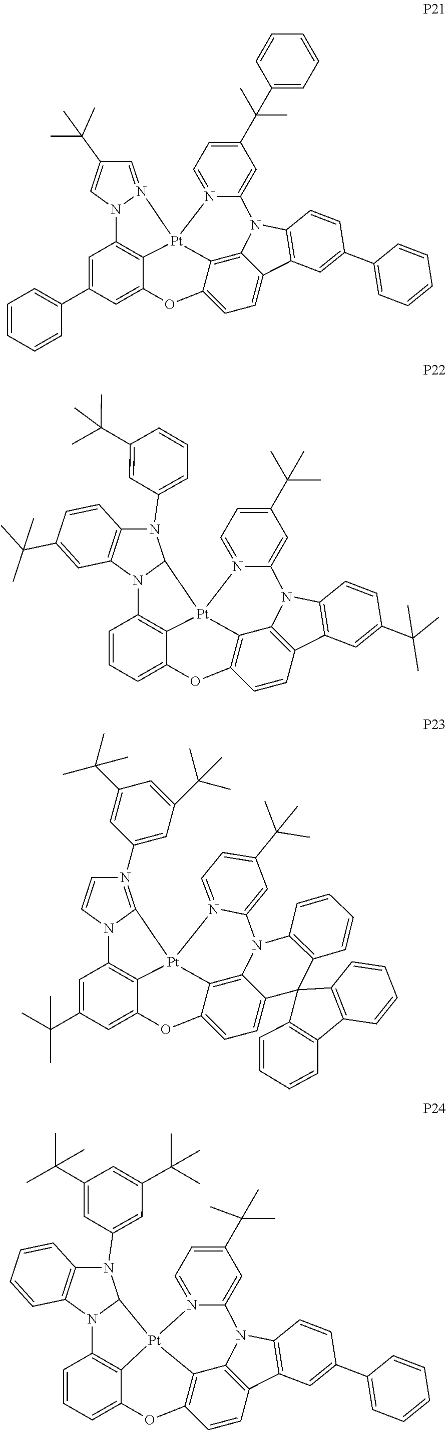

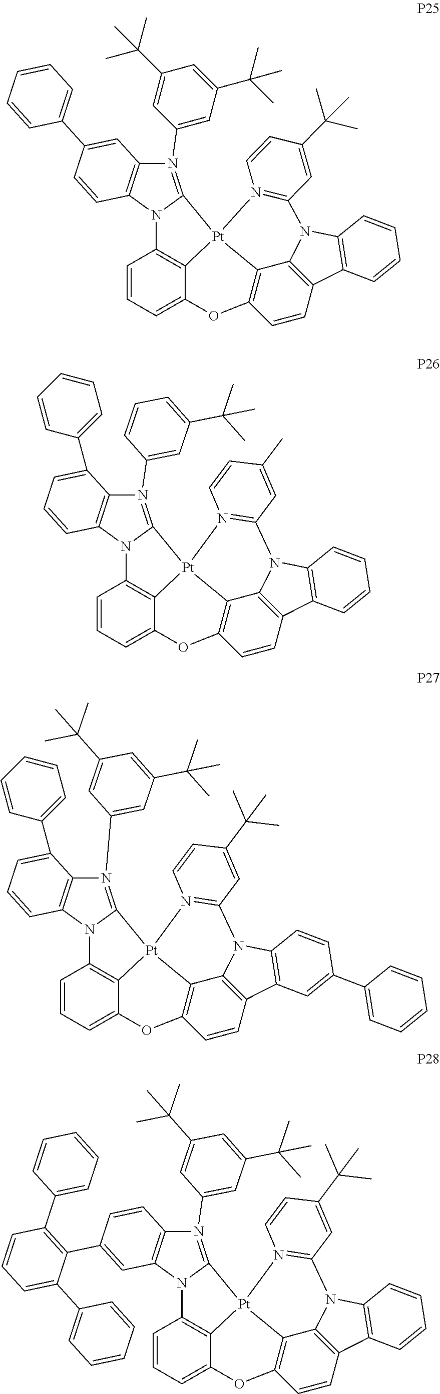

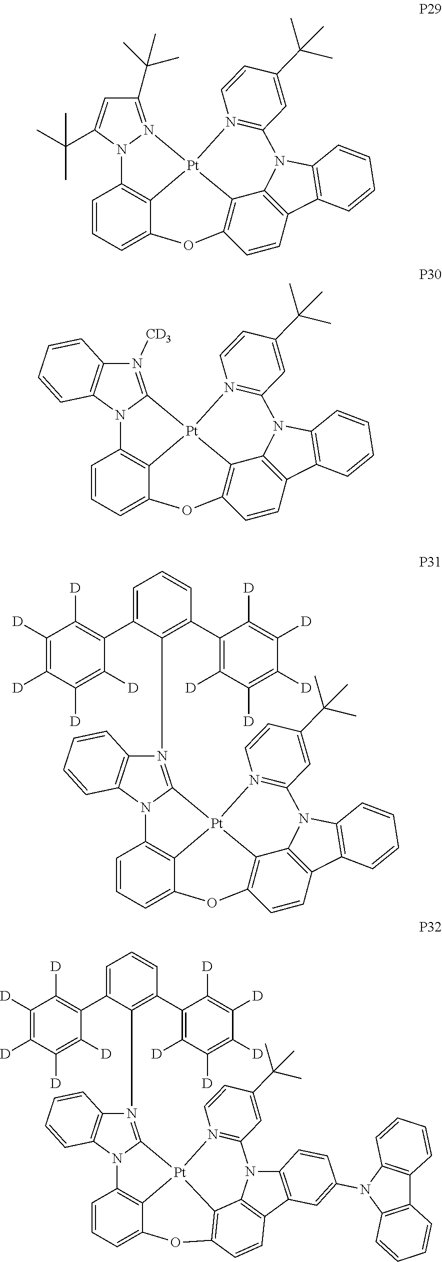

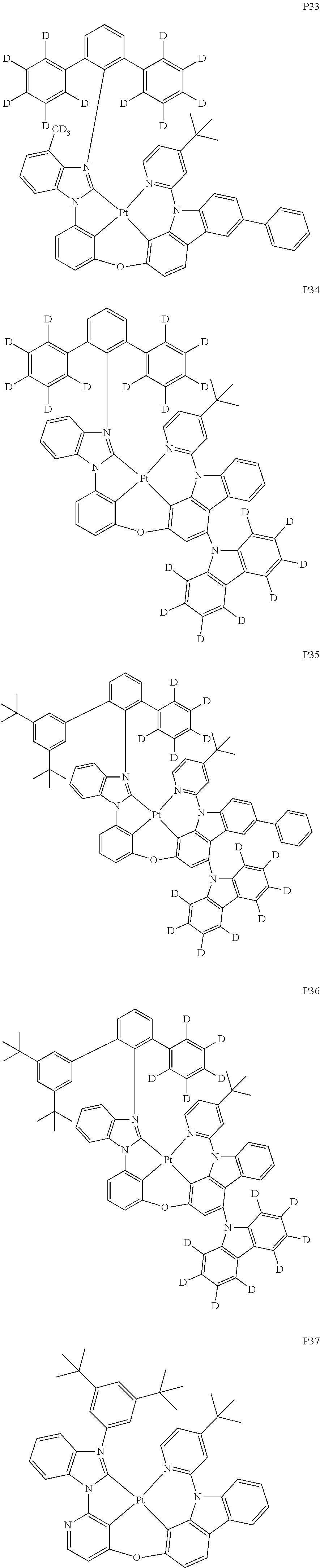

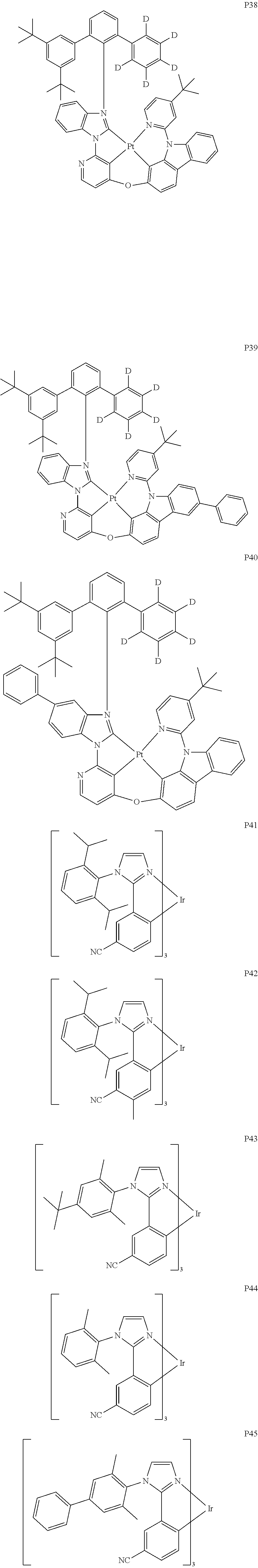

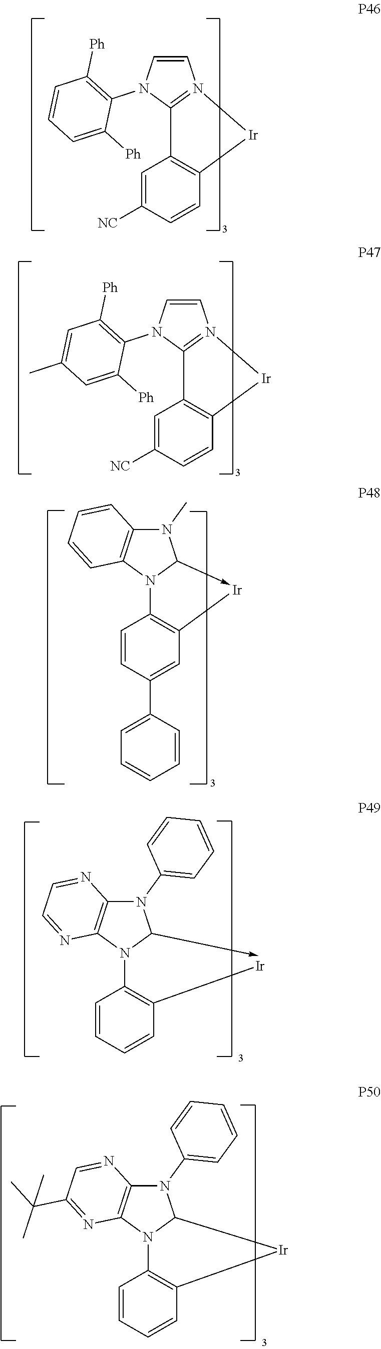

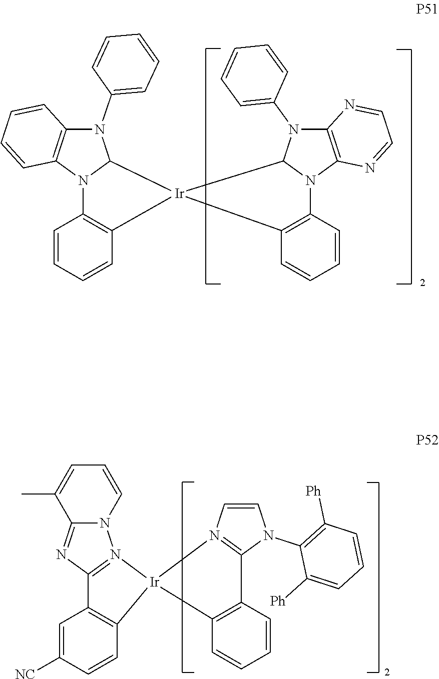

### Description of Formula 4

The emitter as used herein may be a polycyclic compound represented by Formula 4:

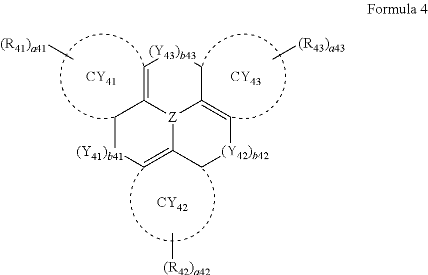

wherein, in Formula 4,
Z may be B or N,
ring CY₄₁ to ring CY₄₃ may each independently be a C₅-C₃₀ carbocyclic group or a C₁-C₃₀ heterocyclic group,
Y₄₁ may be a single bond, *—N(R₄₄)—*′, *—B(R₄₄)—*′, *—P(R₄₄)—*′, *—C(R₄₄)(R₄₅)—*′, *—Si(R₄₄)(R₄₅)-*′, *—Ge(R₄₄)(R₄₅)—*′, *—O—*′, *—S—*′, *—Se—*′, *—C(═O)—*′, or *—S(═O)₂—*′,
Y₄₂ may be a single bond, *—N(R₄₆)—*′, *—B(R₄₆)—*′, *—P(R₄₆)—*′, *—C(R₄₆)(R₄₇)—*′, *—Si(R₄₆)(R₄₇)—*′, *—Ge(R₄₆)(R₄₇)—*′, *—O—*′, *—S—*′, *—Se—*′, *—C(═O)—*′, or *—S(═O)₂—*′,
Y₄₃ may be a single bond, *—N(R₄₈)—*′, *—B(R₄₈)—*′, *—P(R₄₈)—*′, *—C(R₄₈)(R₄₉)—*′, *—Si(R₄₈)(R₄₉)—*′, *—Ge(R₄₈)(R₄₉)—*′, *—O—*′, *—S—*′, *—Se—*′, *—C(═O)—*′, or *—S(═O)₂—*′,
b41 to b43 may each independently be 0 or 1,
when b41 is 0, Y₄₁ may not be present, when b42 is 0, Y₄₂ may not be present, and when b43 is 0, Y₄₃ may not be present,
R₄₁ to R₄₉ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ arylalkylgroup, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ heteroarylalkyl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉),
a41 to a43 may each independently be an integer from 0 to 20,
two or more of R₄₁ (s) in the number of a41 may optionally be bonded to each other to form a C₅-C₃₀ carbocyclic group unsubstituted or substituted with at least one R_{10b} or a C₁-C₃₀ heterocyclic group unsubstituted or substituted with at least one R_{10b},
two or more of R₄₂(s) in the number of a42 may optionally be bonded to each other to form a C₅-C₃₀ carbocyclic group unsubstituted or substituted with at least one R_{10b} or a C₁-C₃₀ heterocyclic group unsubstituted or substituted with at least one R_{10b},
two or more of R₄₃(s) in the number of a43 may optionally be bonded to each other to form a C₅-C₃₀ carbocyclic group unsubstituted or substituted with at least one R_{10b} or a C₁-C₃₀ heterocyclic group unsubstituted or substituted with at least one R_{10b},
two or more of R₄₁ to R₄₉ may optionally be bonded to each other to form a C₅-C₃₀ carbocyclic group unsubstituted or substituted with at least one R_{10b} or a C₁-C₃₀ heterocyclic group unsubstituted or substituted with at least one R_{10b},
R_{10b} is as described for R₄₁,
* and *′ each indicate a binding site to a neighboring atom,
a substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₇-C₆₀ arylalkylgroup, the substituted C₆-C₆₀ aryloxy group, the substituted C₆-C₆₀ arylthio group, the substituted C₁-C₆₀ heteroaryl group, the substituted C₂-C₆₀ heteroarylalkyl group, the substituted monovalent non-aromatic condensed polycyclic group, and the substituted monovalent non-aromatic condensed heteropolycyclic group may be:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or a combination thereof;
a C₃-C₁₀ cycloalkyl group, a C₁-C₁ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁ cycloalkyl group, a C₁-C₁ heterocycloalkyl group, a C₃-C₁₀ cycloalkenyl group, a C₂-C₁ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉), or a combination thereof;
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉); or
a combination thereof,
wherein Q₁ to Q₉, Q₁₁ to Q₁₉, Q₂₁ to Q₂₉, and Q₃₁ to Q₃₉ are each independently hydrogen; deuterium; —F; —Cl; —Br; —I; a hydroxyl group; a cyano group; a nitro group; an amidino group; a hydrazine group; a hydrazone group; a carboxylic acid group or a salt thereof; a sulfonic acid group or a salt thereof; a phosphoric acid group or a salt thereof; or a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, unsubstituted or substituted with deuterium, a C₁-C₆₀ alkyl group, a C₆-C₆₀ aryl group, or a combination thereof.

In an embodiment, rings CY₄₁ to CY₄₃ may each independently be i) a benzene group or ii) a polycyclic group in which two or more C₃-C₆₀ cyclic groups are condensed with each other. At least two C₃-C₆₀ cyclic groups in the polycyclic group may be condensed with each other while sharing boron (B) or nitrogen (N).

In one or more embodiments, at least one of b41 to b43 or at least two of b41 to b43 may each be 1. In an embodiment, two of b41 to b43 may be 1, and the other may be 0.

In one or more embodiments, R₄₁ to R₄₉ may each independently be:
hydrogen, deuterium, —F, —Cl, —Br, —I, a hydroxyl group, a cyano group (CN), a nitro group, an amino group, a C₁-C₆₀ alkyl group, or a C₁-C₆₀ alkoxy group;
a C₁-C₆₀ alkyl group or a C₁-C₆₀ alkoxy group, each substituted with at least one of deuterium, —F, —Cl, —Br, —I, a hydroxyl group, a cyano group (CN), a nitro group, an amino group, a phenyl group, a biphenyl group, a naphthyl group, a fluorenyl group, a phenanthrenyl group, an anthracenyl group, a fluoranthenyl group, a triphenylenyl group, a pyrenyl group, and a chrysenyl group; or
a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, or a carbazolyl group, each unsubstituted or substituted with at least one of deuterium, —F, —Cl, —Br, —I, a hydroxyl group, a cyano group (CN), a nitro group, an amino group, a C₁-C₆₀ alkyl group, a C₁-C₆₀ alkoxy group, a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, and a carbazolyl group.

In one or more embodiments, the polycyclic compound represented by Formula 4 may be represented by one of Formulae 4-1 to 4-9:

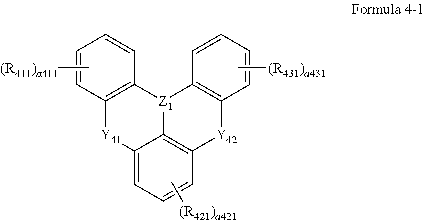

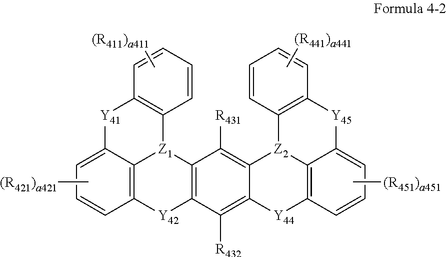

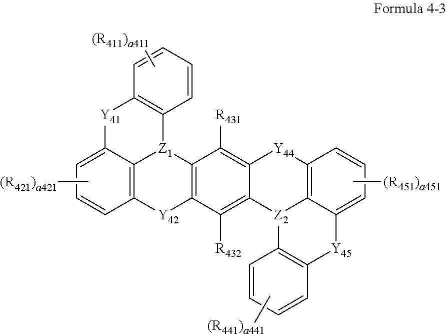

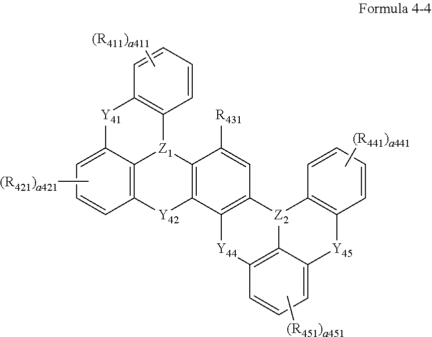

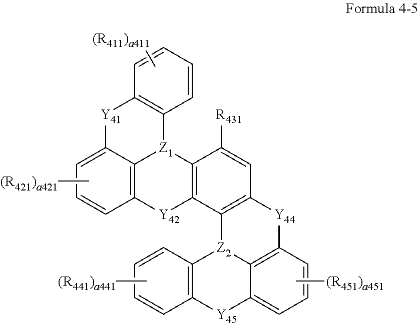

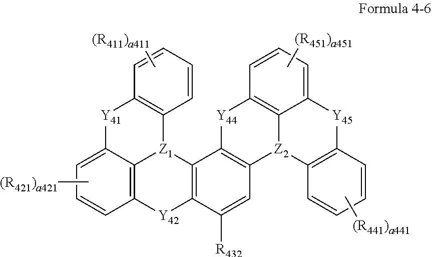

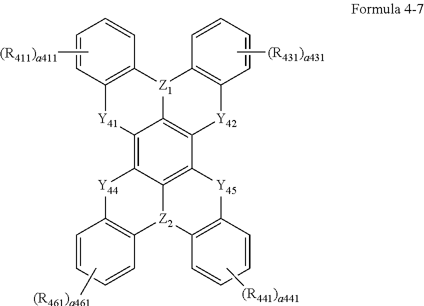

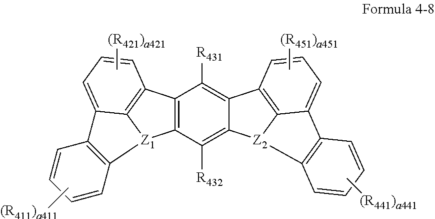

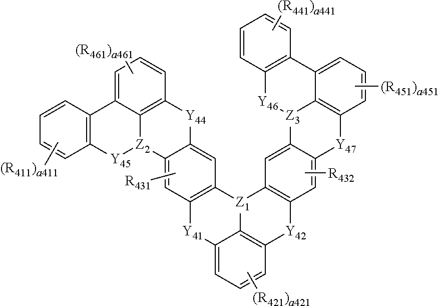

wherein, in Formulae 4-1 to 4-9,
Z₁ to Z₃ may each be as described for Z,
Y₄₁ and Y₄₂ may each be as described herein,
Y₄₄ to Y₄₇ may respectively be as described for Y₄₁ and Y₄₂,
R₄₁₁ may be as described for R₄₁, R₄₂₁ may be as described for R₄₂, R₄₃₁ and R₄₃₂ may each be as described for R₄₃, R₄₄₁ may be as described for R₄₁, R₄₅₁ may be as described for R₄₂, and R₄₆₁ may be as described for R₄₃,
a411 may be an integer from 0 to 4,
a421 may be an integer from 0 to 3,
a431 may be an integer from 0 to 4,
a441 may be an integer from 0 to 4,
a451 may be an integer from 0 to 3, and
a461 may be an integer from 0 to 3.

Example of polycyclic compound represented by Formula 4

The polycyclic compound represented by Formula 4 may be selected from Compounds, D1 to D30:

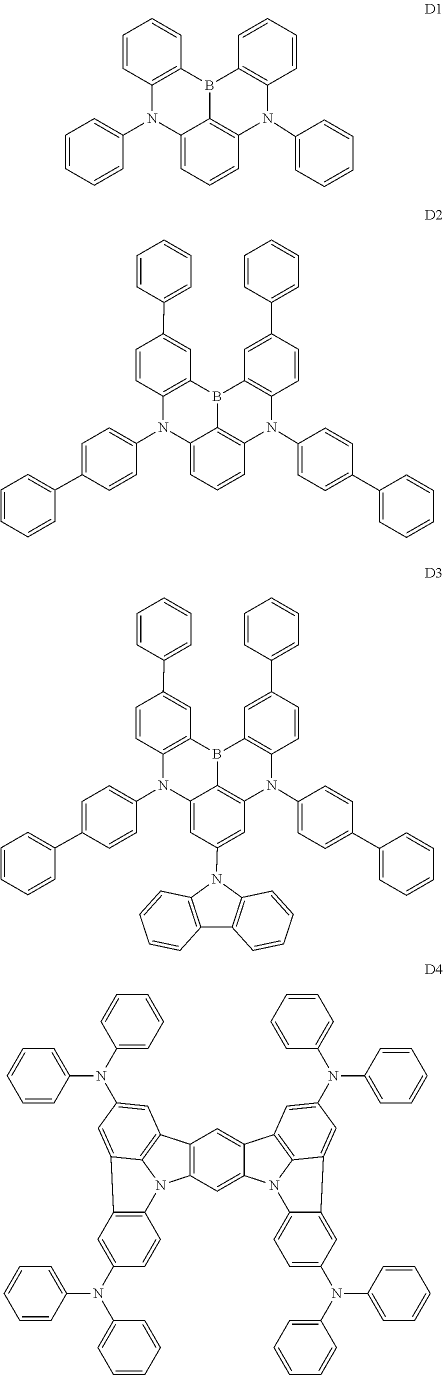 [SMILES: c1ccc(N2c3ccccc3B3c4ccccc4N(c4ccccc4)c4cccc2c43)cc1 ; c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-n6c7ccccc7c7ccccc76)cc3c54)cc2)cc1 ; c1ccc(N(c2ccccc2)c2ccc3c(c2)c2cc(N(c4ccccc4)c4ccccc4)cc4c5cc6c7cc(N(c8ccccc8)c8ccccc8)cc8c9cc(N(c%10ccccc%10)c%10ccccc%10)ccc9n(c6cc5n3c24)c87)cc1]

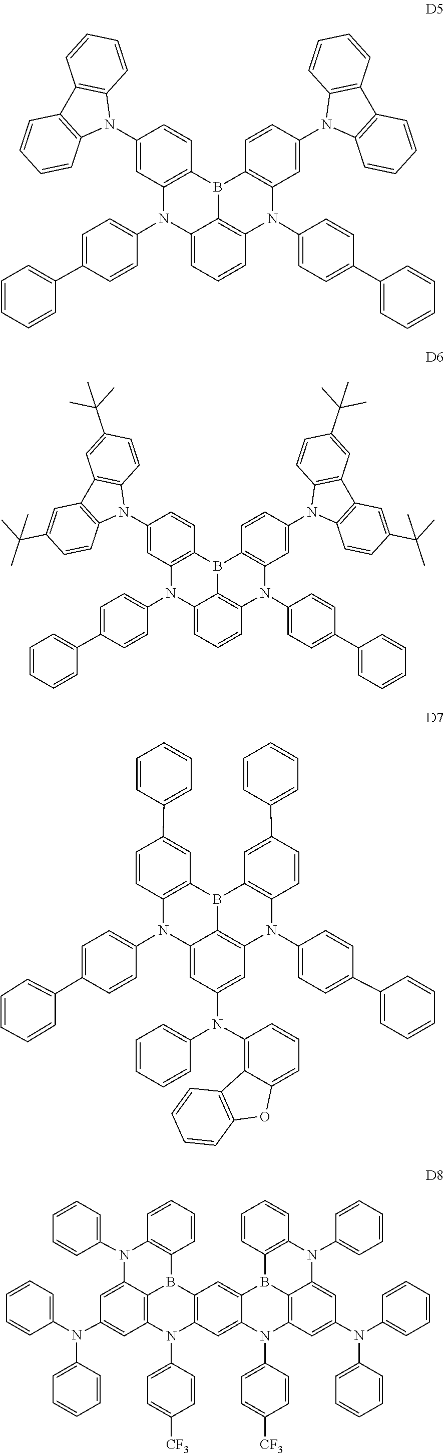

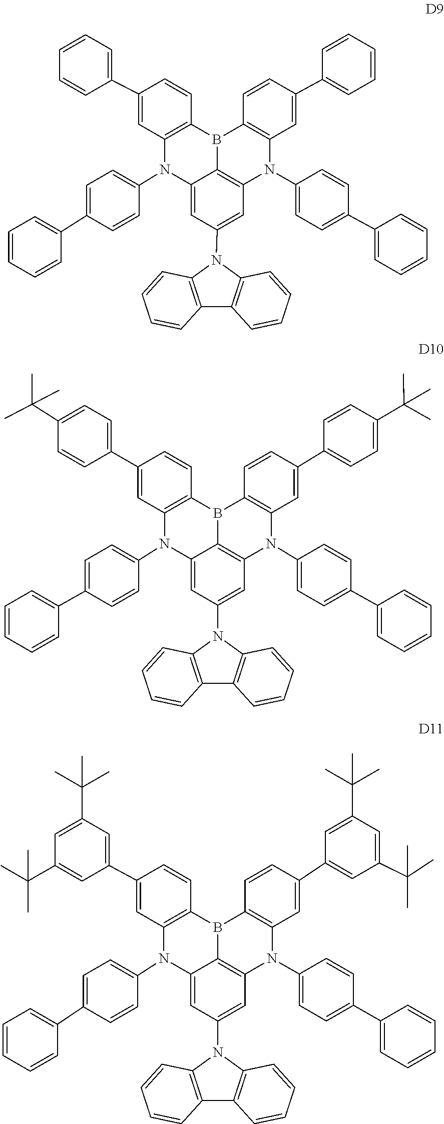 [SMILES: c1ccc(-c2ccc(N3c4cc(-c5ccccc5)ccc4B4c5ccc(-c6ccccc6)cc5N(c5ccc(-c6ccccc6)cc5)c5cc(-n6c7ccccc7c7ccccc76)cc3c54)cc2)cc1 ; CC(C)(C)c1ccc(-c2ccc3c(c2)N(c2ccc(-c4ccccc4)cc2)c2cc(-n4c5ccccc5c5ccccc54)cc4c2B3c2ccc(-c3ccc(C(C)(C)C)cc3)cc2N4c2ccc(-c3ccccc3)cc2)cc1 ; CC(C)(C)c1cc(-c2ccc3c(c2)N(c2ccc(-c4ccccc4)cc2)c2cc(-n4c5ccccc5c5ccccc54)cc4c2B3c2ccc(-c3cc(C(C)(C)C)cc(C(C)(C)C)c3)cc2N4c2ccc(-c3ccccc3)cc2)cc(C(C)(C)C)c1]

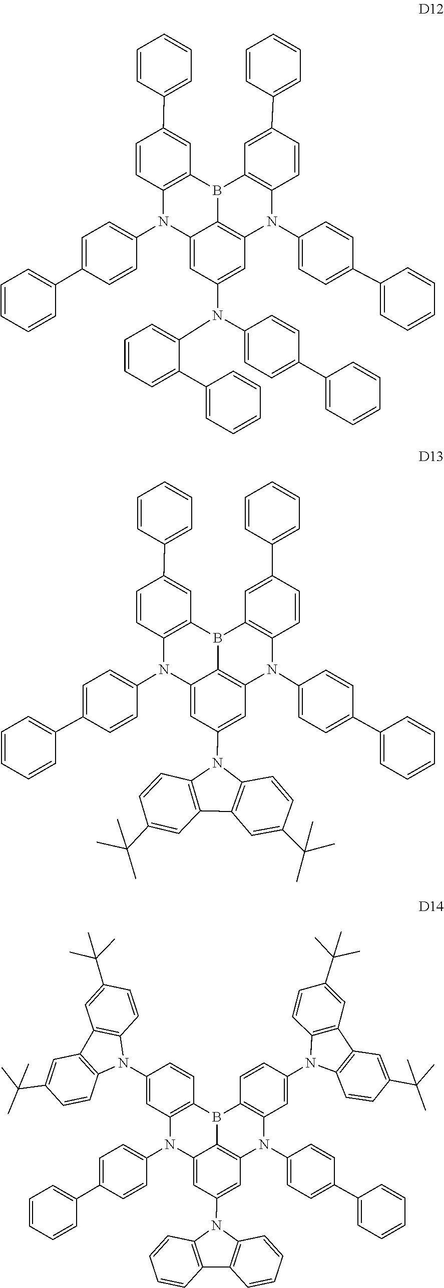 [SMILES: c1ccc(-c2ccc(N(c3cc4c5c(c3)N(c3ccc(-c6ccccc6)cc3)c3ccc(-c6ccccc6)cc3B5c3cc(-c5ccccc5)ccc3N4c3ccc(-c4ccccc4)cc3)c3ccccc3-c3ccccc3)cc2)cc1 ; CC(C)(C)c1ccc2c(c1)c1cc(C(C)(C)C)ccc1n2-c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ccc(-n2c4ccc(C(C)(C)C)cc4c4cc(C(C)(C)C)ccc42)cc1N3c1ccc(-c2ccccc2)cc1]

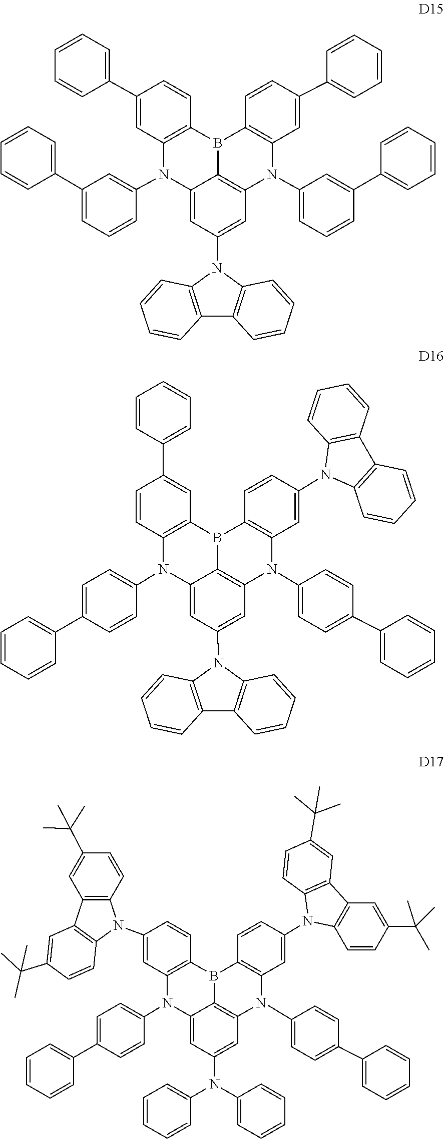

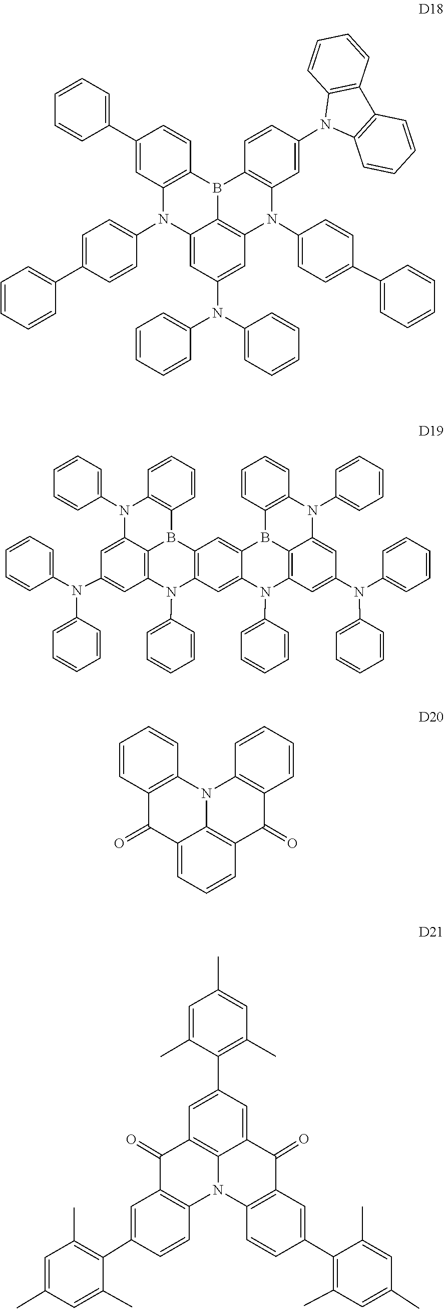

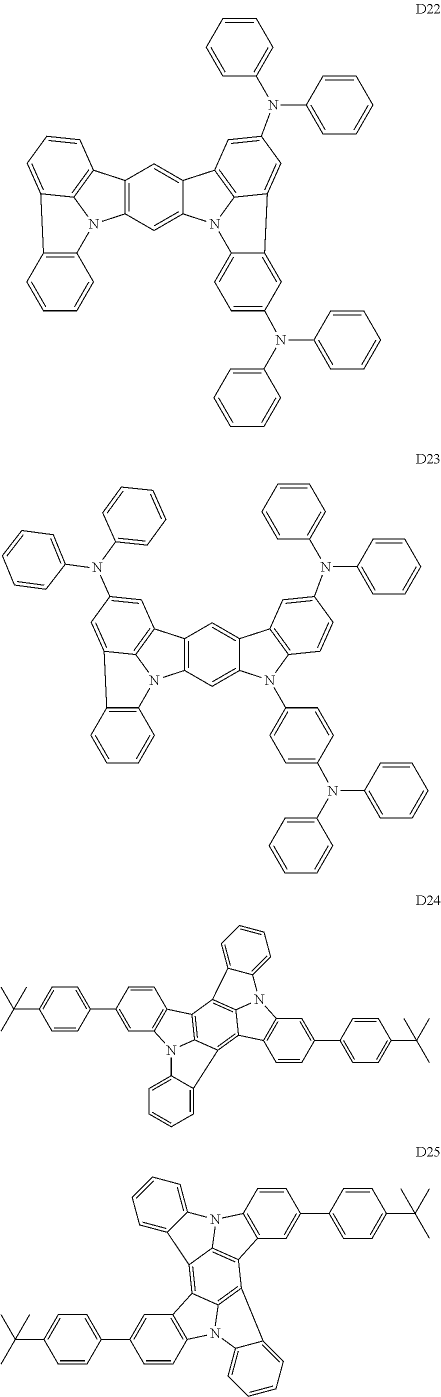

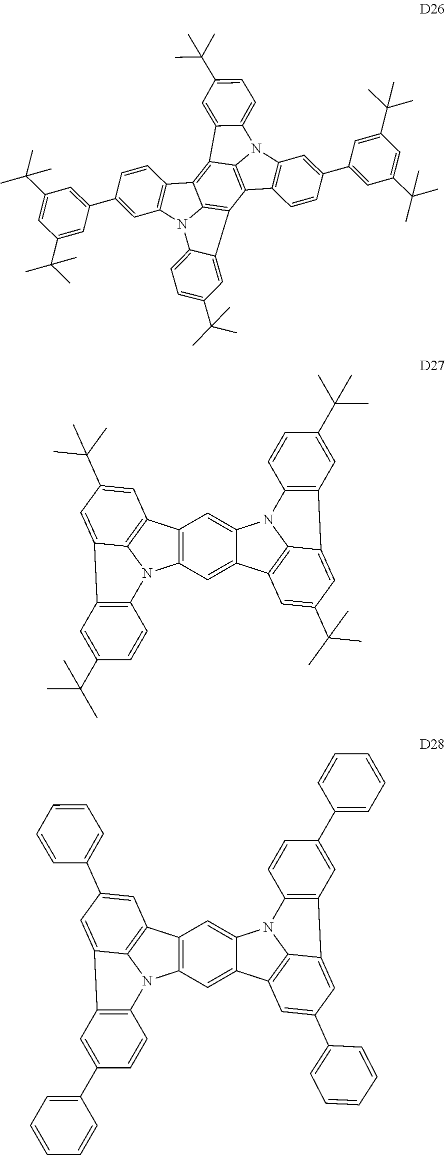

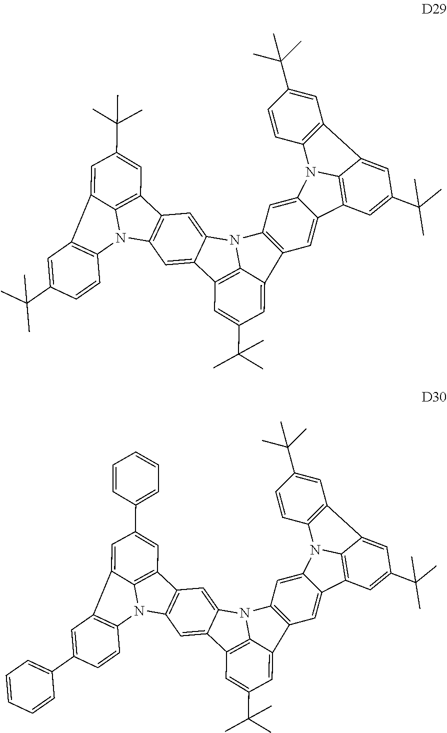 [SMILES: CC(C)(C)c1ccc2c(c1)c1cc(C(C)(C)C)cc3c4cc5c(cc4n2c13)c1cc(C(C)(C)C)cc2c3cc4c6cc(C(C)(C)C)cc7c8cc(C(C)(C)C)ccc8n(c4cc3n5c12)c76 ; CC(C)(C)c1ccc2c(c1)c1cc(C(C)(C)C)cc3c4cc5c6cc(C(C)(C)C)cc7c8cc9c(cc8n(c5cc4n2c13)c76)c1cc(-c2ccccc2)cc2c3cc(-c4ccccc4)ccc3n9c21]

### Layer

In an embodiment, a layer may include the composition described herein.

For example, when the composition further includes an emitter, the layer may be an emission layer.

### Light-Emitting Device

In one or more embodiments, a light-emitting device, for example, an organic light-emitting device may include: a first electrode; a second electrode; and an interlayer arranged between the first electrode and the second electrode and including an emission layer, wherein the interlayer may include the composition as described herein.

In an embodiment, the composition may be included in the emission layer of the light-emitting device. The emission layer may emit blue light. For example, the emission layer may emit blue light having a maximum emission wavelength range of about 400 nm to about 490 nm, about 410 nm to about 470 nm, or about 420 nm to about 450 nm.

Because the organic light-emitting device includes the composition as described above, the organic light-emitting device may have excellent luminescence efficiency and lifespan characteristics.

The first electrode may be an anode, which is a hole injection electrode, and the second electrode may be a cathode, which is an electron injection electrode; or the first electrode may be a cathode, which is an electron injection electrode, and the second electrode may be an anode, which is a hole injection electrode.

For example, in the organic light-emitting device, the first electrode may be an anode, the second electrode may be a cathode, and the interlayer may further include a hole transport region between the first electrode and the emission layer and an electron transport region between the emission layer and the second electrode, wherein the hole transport region may include a hole injection layer, a hole transport layer, an electron blocking layer, an auxiliary layer, or a combination thereof, and the electron transport region may include a buffer layer, a hole blocking layer, an electron transport layer, an electron injection layer, or a combination thereof.

The term “interlayer” as used herein refers to a single layer and/or a plurality of layers arranged between the first electrode and the second electrode of the organic light-emitting device. The “interlayer” may include not only organic compounds but also organometallic complexes including metals.

### DESCRIPTION OF THE FIGURE

The FIGURE is a schematic cross-sectional view of an organic light-emitting device 10 according to an embodiment. Hereinafter, the structure and manufacturing method of the organic light-emitting device 10 according to an embodiment of the disclosure will be described in connection with FIGURE.

In the FIGURE, an organic light-emitting device 10 includes a first electrode 11, a second electrode 19 facing the first electrode 11, and an interlayer 10A between the first electrode 11 and the second electrode 19.

In the FIGURE, the interlayer 10A includes an emission layer 15, a hole transport region 12 is between the first electrode 11 and an emission layer 15, and an electron transport region 17 is between the emission layer 15 and the second electrode 19.

A substrate may be additionally disposed under the first electrode 11 or on the second electrode 19. The substrate may be a conventional substrate used in organic light-emitting devices, e.g., a glass substrate or a transparent plastic substrate, each having excellent mechanical strength, thermal stability, transparency, surface smoothness, ease of handling, and water repellency.

First electrode 11

The first electrode 11 may be produced by depositing or sputtering, onto the substrate, a material for forming the first electrode 11. The first electrode 11 may be an anode. The material for forming the first electrode 11 may include materials with a high work function to facilitate hole injection.

The first electrode 11 may be a reflective electrode, a semi-transmissive electrode, or a transmissive electrode. When the first electrode 11 is a transmissive electrode, the material for forming the first electrode 11 may include indium tin oxide (ITO), indium zinc oxide (IZO), tin oxide (SnO₂), zinc oxide (ZnO), or a combination thereof. In some embodiments, when the first electrode 11 is a semi-transmissive electrode or a reflective electrode, a material for forming the first electrode 11 may include magnesium (Mg), silver (Ag), aluminum (Al), aluminum-lithium (Al—Li), calcium (Ca), magnesium-indium (Mg—In), magnesium-silver (Mg—Ag), or a combination thereof.

The first electrode 11 may have a single-layered structure or a multi-layered structure including a plurality of layers.

### Emission Layer 15

A thickness of the emission layer 15 may be in a range of about 100 angstroms (Å) to about 1,000 Å, for example, about 200 Å to about 600 Å. When the thickness of the emission layer is within these ranges, excellent light-emission characteristics may be obtained without a substantial increase in driving voltage.

In an embodiment, the emission layer 15 may include the composition as described herein.

### Hole Transport Region 12

The hole transport region 12 may be arranged between the first electrode 11 and the emission layer 15 of the organic light-emitting device 10.

The hole transport region 12 may have a single-layered structure or a multi-layered structure.

For example, the hole transport region 12 may have a hole injection layer, a hole transport layer, a hole injection layer/hole transport layer structure, a hole injection layer/first hole transport layer/second hole transport layer structure, a hole injection layer/first hole transport layer/second hole transport layer/electron blocking layer structure, a hole transport layer/organic layer structure, a hole injection layer/hole transport layer/organic layer structure, a hole transport layer/electron blocking layer structure, or a hole injection layer/hole transport layer/electron blocking layer structure.

The hole transport region 12 may include any compound having hole transporting properties.

For example, the hole transport region 12 may include an amine-based compound.

In an embodiment, the hole transport region 12 may include, for example, m-MTDATA, TDATA, 2-TNATA, NPB, β-NPB, TPD, Spiro-TPD, Spiro-NPB, methylated NPB, TAPC, HMTPD, 4,4′,4″-tris(N-carbazolyl)triphenylamine (“TCTA”), polyaniline/dodecylbenzenesulfonic acid (“PANI”/“DBSA”), poly(3,4-ethylenedioxythiophene)/poly(4-styrenesulfonate) (“PEDOT”/“PSS”), polyaniline/camphor sulfonic acid (“PANI”/“CSA”), polyaniline/poly(4-styrenesulfonate) (“PANI”/“PSS”), one of a compound represented by Formula 201 to a compound represented by Formula 205, or a combination thereof:

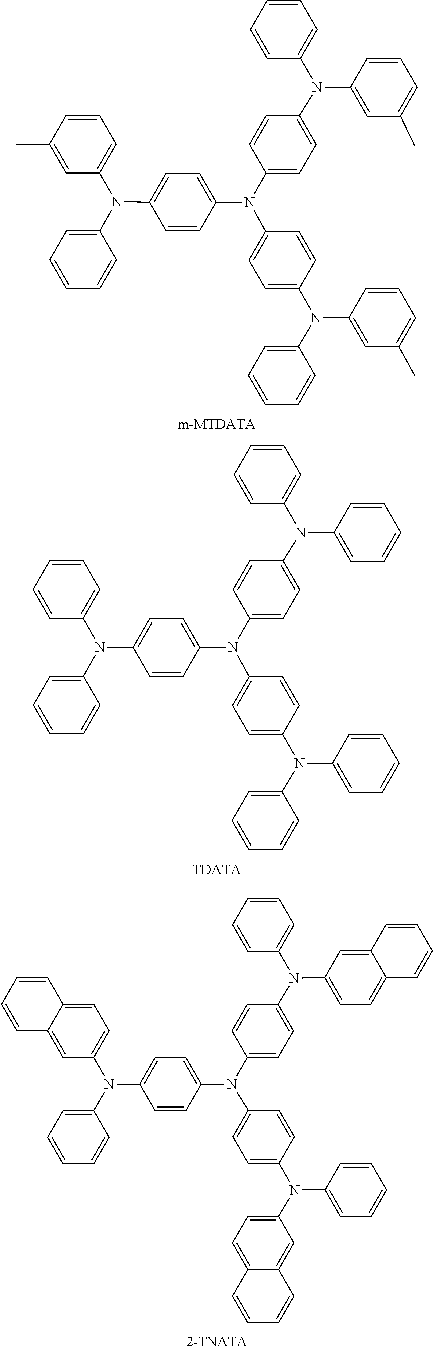

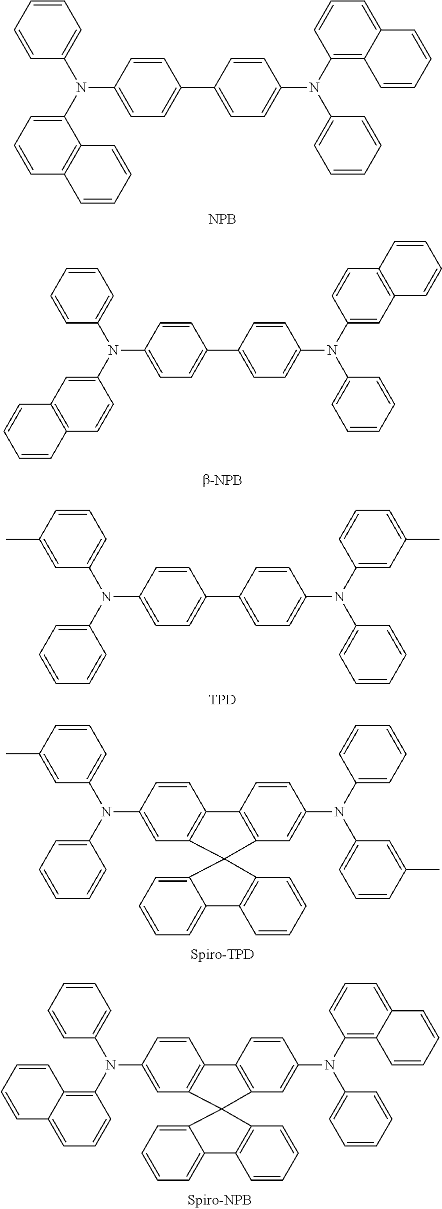

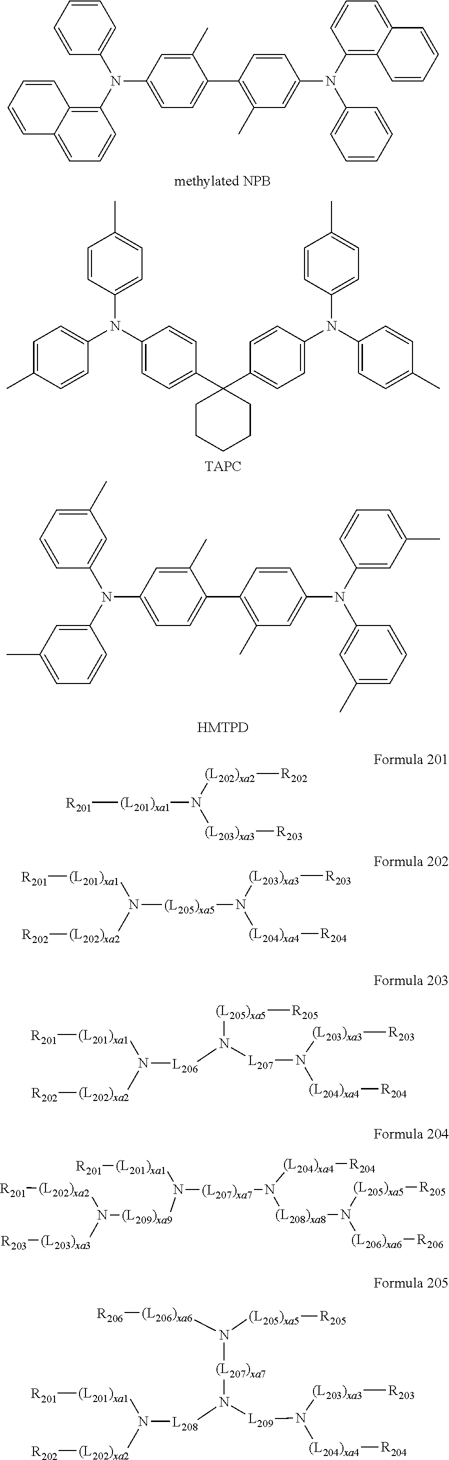

wherein, in Formulae 201 to 205,
L₂₀₁ to L₂₀₉ may each independently be *—O—*′, *—S—*′, a substituted or unsubstituted C₅-C₆₀ carbocyclic group, or a substituted or unsubstituted C₁-C₆₀ heterocyclic group,
xa1 to xa9 may each independently be an integer from 0 to 5,
R₂₀₁ to R₂₀₆ may each independently be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group, wherein neighboring two groups of R₂₀₁ to R₂₀₆ may optionally be linked to each other via a single bond, a dimethyl-methylene group, or a diphenyl-methylene group.

For example, L₂₀₁ to L₂₀₉ may be a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, a hexacene group, a pentacene group, a rubicene group, a coronene group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, or a triindolobenzene group, each unsubstituted or substituted with deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a quaterphenyl group, —Si(Q₁₁)(Q₁₂)(Q₁₃), or a combination thereof, xa1 to xa9 may each independently be 0, 1, or 2, and R₂₀₁ to R₂₀₆ may each independently be a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an indeno carbazolyl group, an indolocarbazolyl group, a benzofurocarbazolyl group, or a benzothienocarbazolyl group, each unsubstituted or substituted with deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, —Si(Q₃₁)(Q₃₂)(Q₃₃), —N(Q₃₁)(Q₃₂), or a combination thereof.

Q₁₁ to Q₁₃ and Q₃₁ to Q₃₃ may each independently be a C₁-C₁₀ alkyl group, a C₁-C₁₀ alkoxy group, a phenyl group, a biphenyl group, a terphenyl group, or a naphthyl group.

In one or more embodiments, the hole transport region 12 may include a carbazole-containing amine-based compound.

In an embodiment, the hole transport region 12 may include a carbazole-containing amine-based compound and a carbazole-free amine-based compound.

The carbazole-containing amine-based compound may include, for example, compounds represented by Formula 201 including a carbazole group and further including at least one of a dibenzofuran group, a dibenzothiophene group, a fluorene group, a spiro-bifluorene group, an indenocarbazole group, an indolocarbazole group, a benzofurocarbazole group, and a benzothienocarbazole group.

The carbazole-free amine-based compound may include, for example, compounds represented by Formula 201 not including a carbazole group and including at least one of a dibenzofuran group, a dibenzothiophene group, a fluorene group, a spiro-bifluorene group, an indenocarbazole group, an indolocarbazole group, a benzofurocarbazole group, and a benzothienocarbazole group.

In one or more embodiments, the hole transport region 12 may include a compound represented by Formula 201, a compound represented by Formula 202, or a combination thereof.

In an embodiment, the hole transport region 12 may include a compound represented by Formula 201-1, 202-1, or 201-2, or a combination thereof:

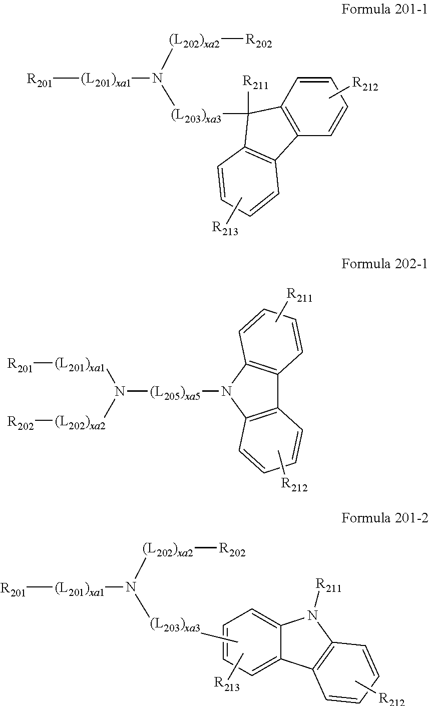

wherein, in Formulae 201-1, 202-1, and 201-2, L₂₀₁ to L₂₀₃, L₂₀₅, xa1 to xa3, xa5, R₂₀₁, and R₂₀₂ may respectively be as described for L₂₀₁ to L₂₀₃, L₂₀₅, xa1 to xa3, xa5, R₂₀₁, and R₂₀₂, and R₂₁₁ to R₂₁₃ may each independently be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a phenyl group substituted with a C₁-C₁₀ alkyl group, a phenyl group substituted with —F, a naphthyl group, a fluorenyl group, a spiro-bifluorenyl group, a dimethylfluorenyl group, a diphenylfluorenyl group, a triphenylenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, or a pyridinyl group.

For example, the hole transport region 12 may include one of Compounds HT1 to HT39 or a combination thereof:

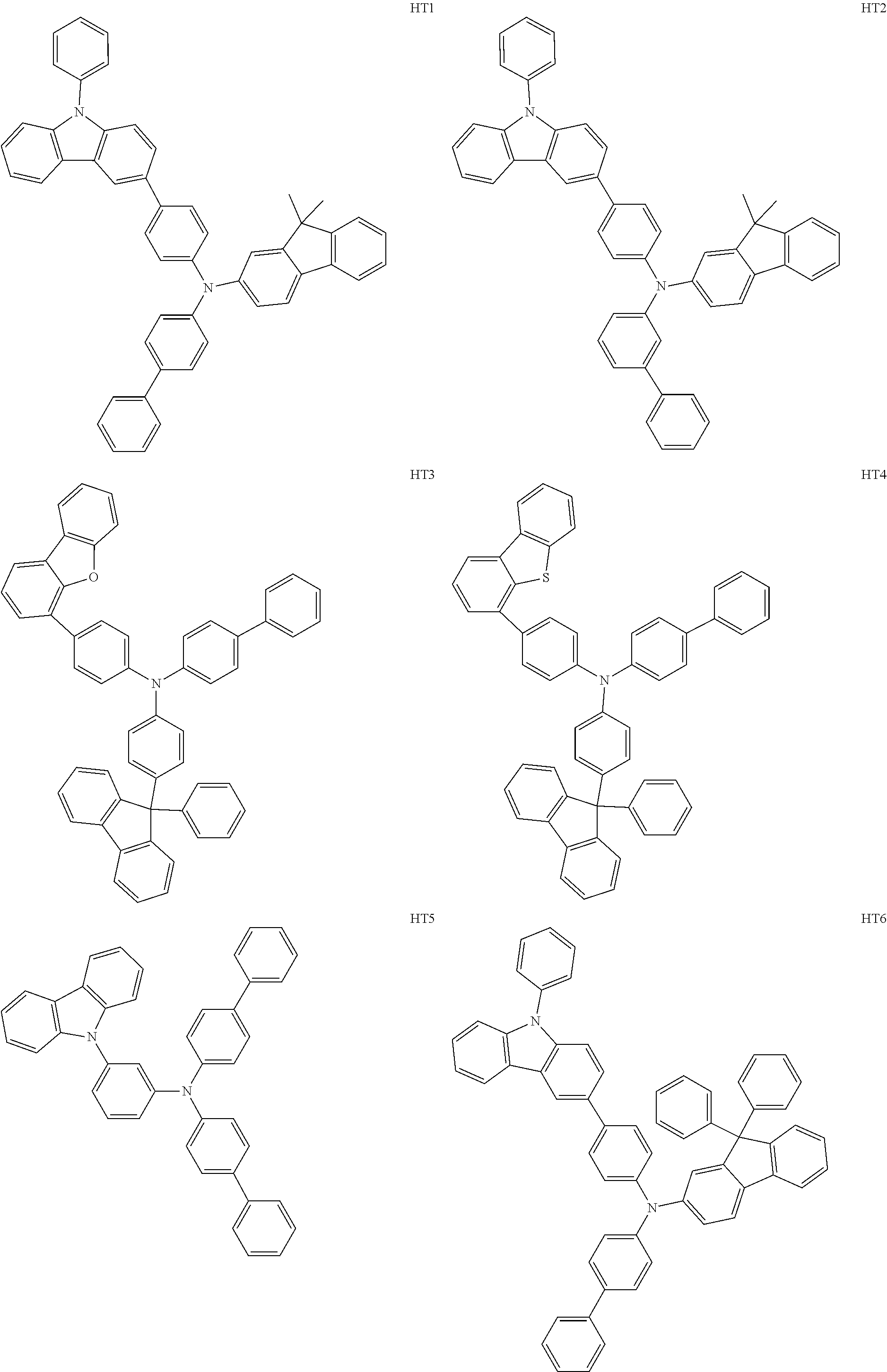

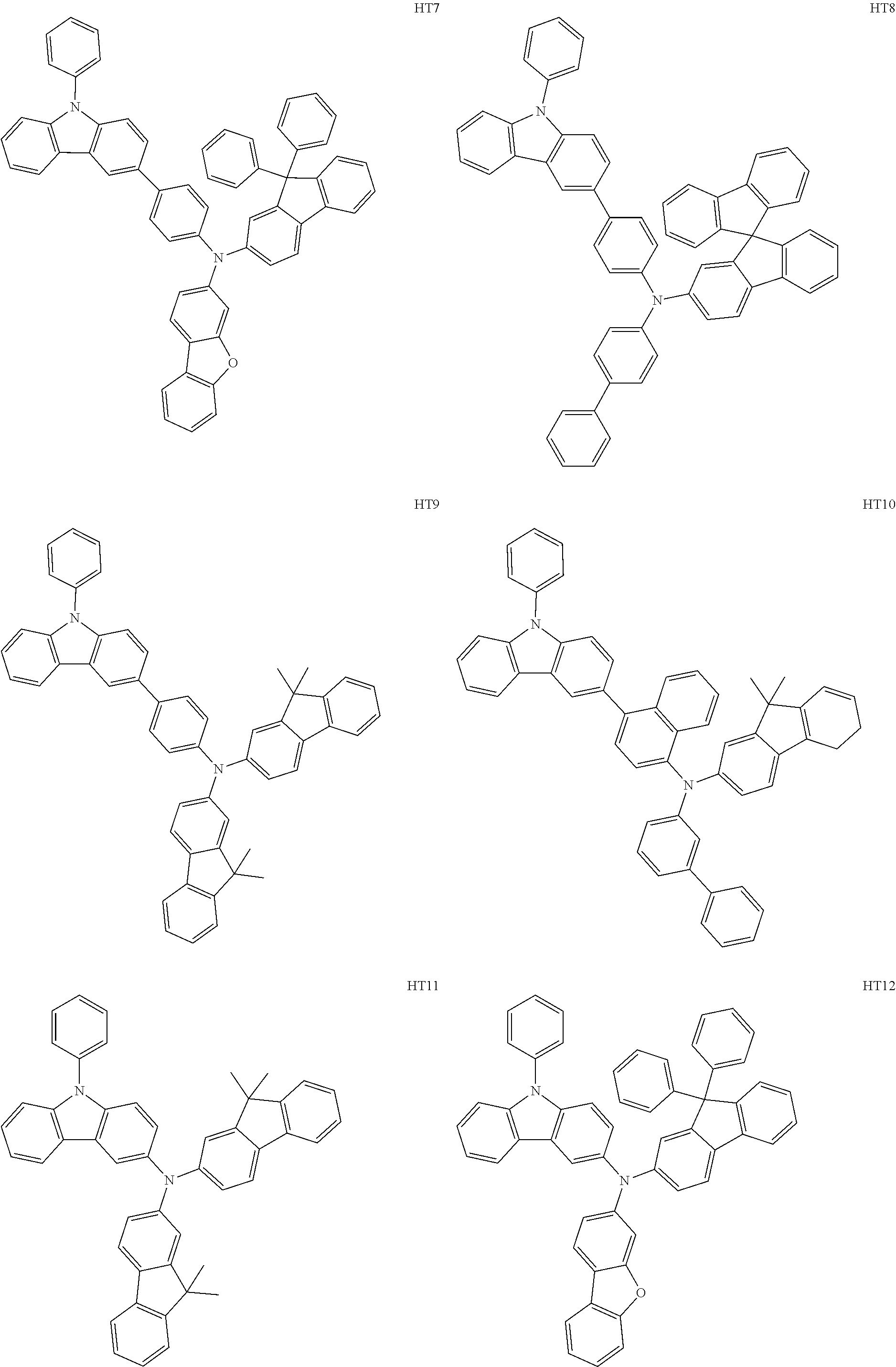

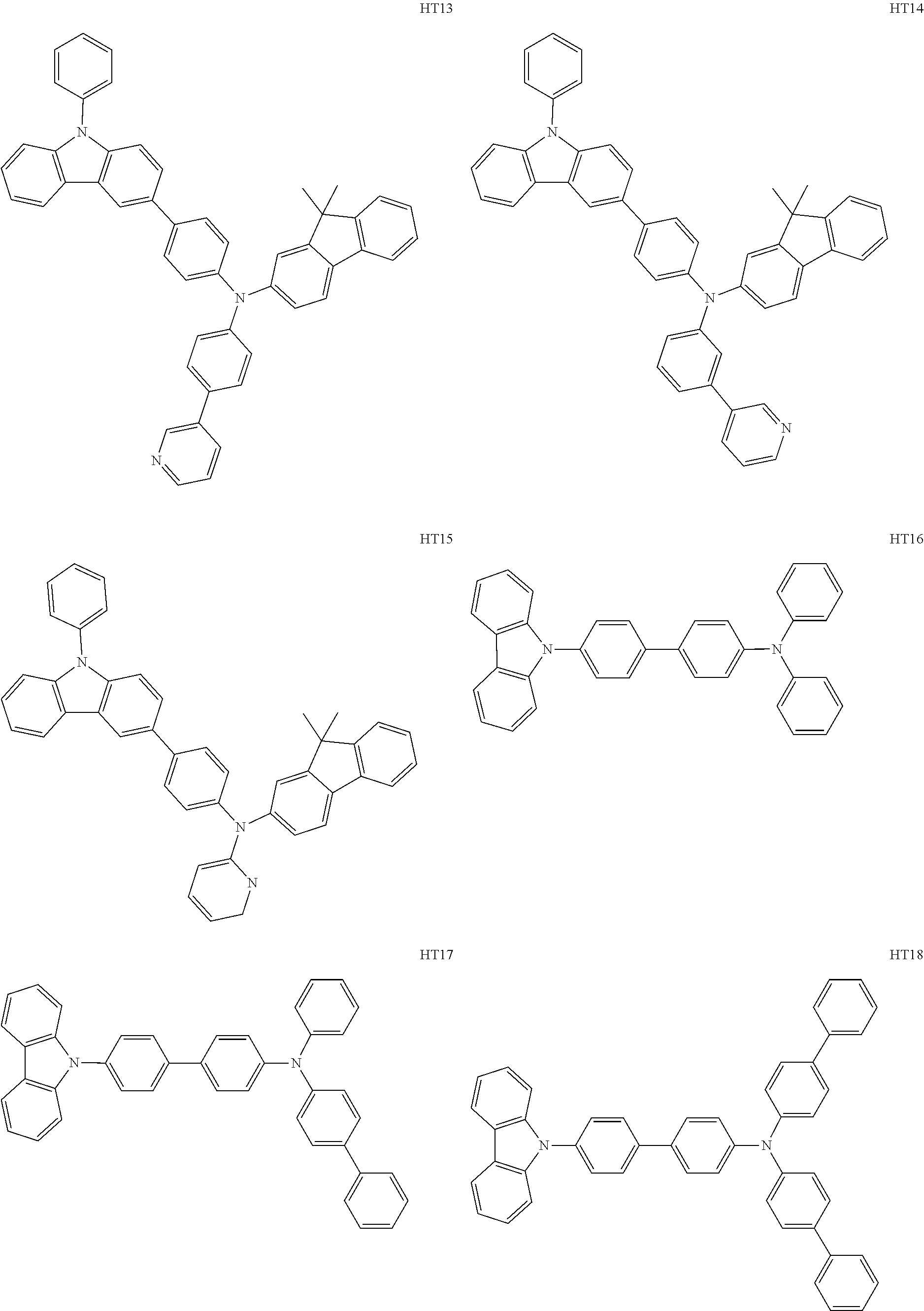

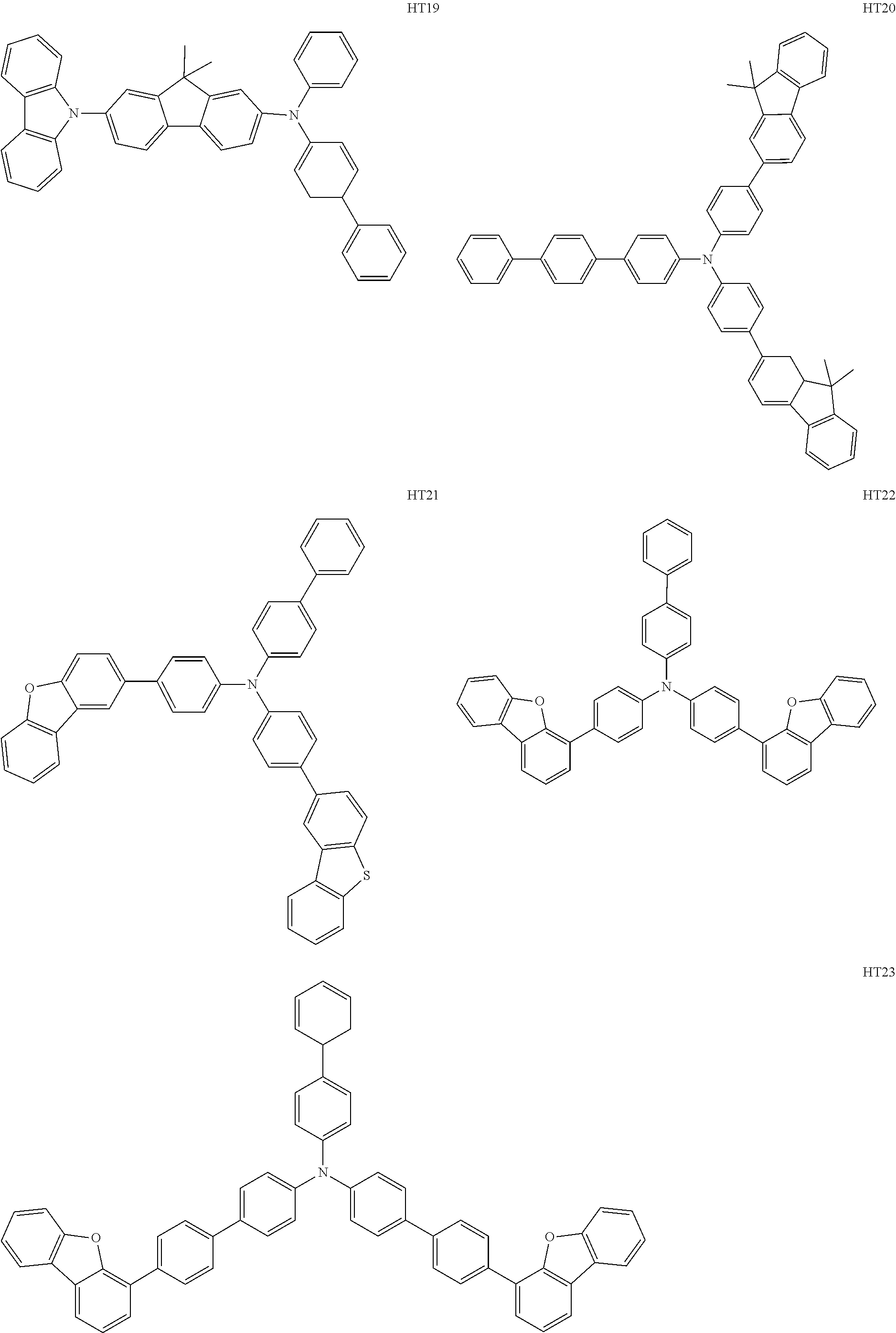

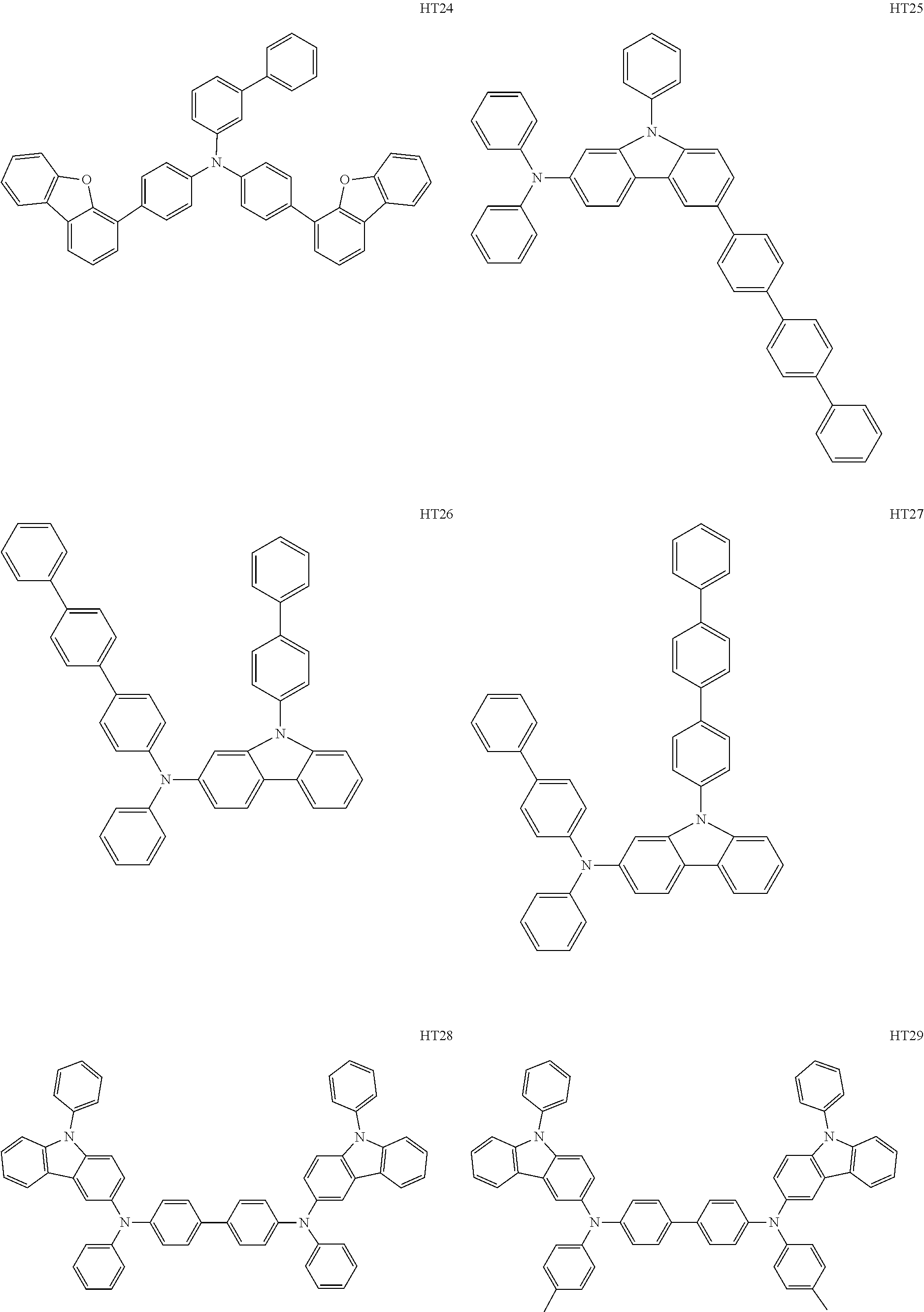

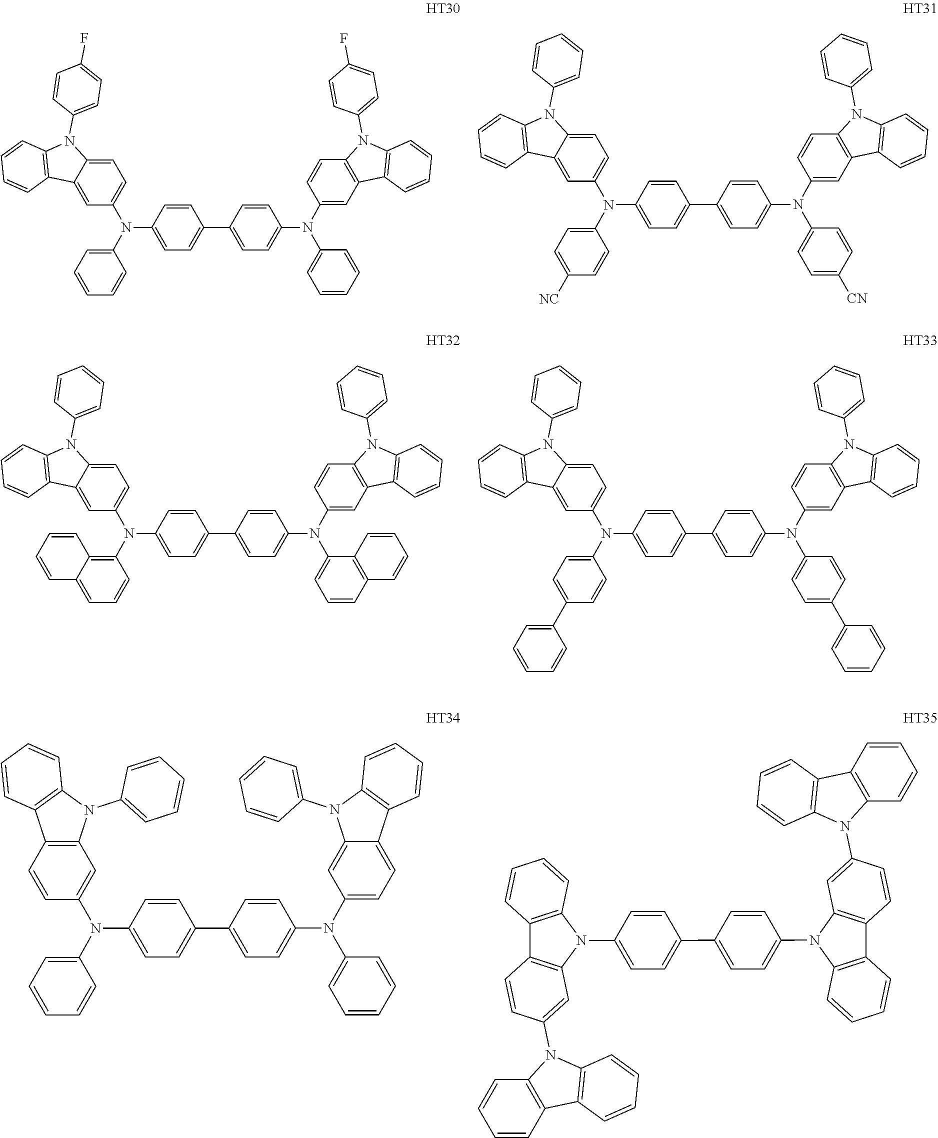

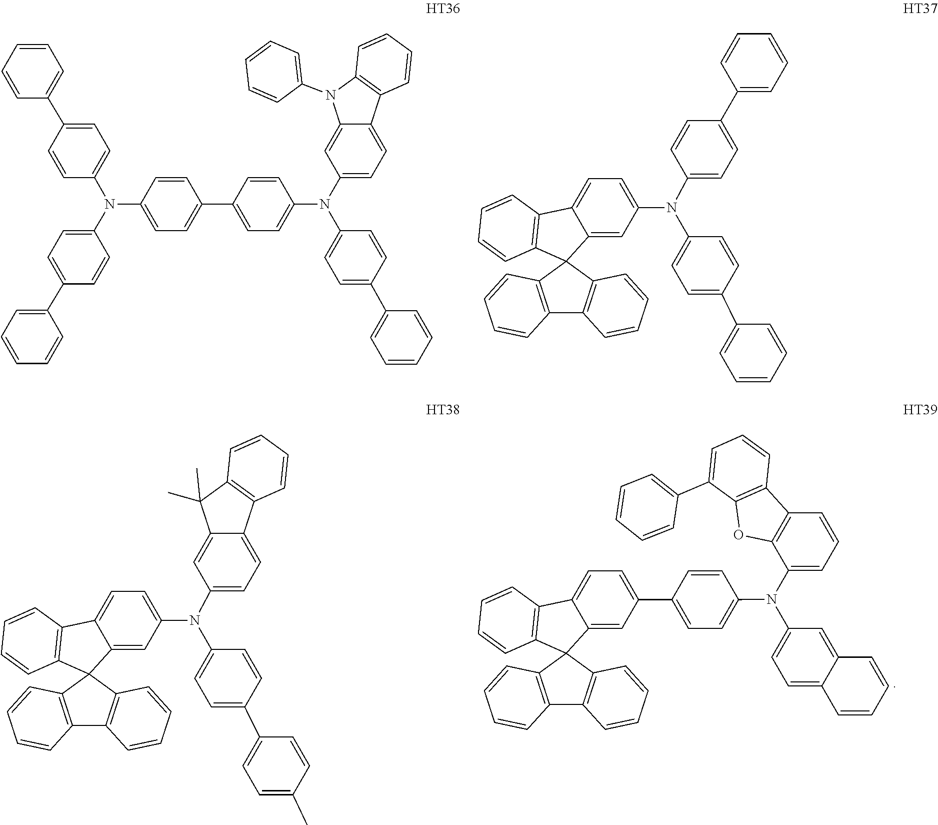 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

In one or more embodiments, hole transport region 12 of the organic light-emitting device 10 may further include a p-dopant. When the hole transport region 12 further includes a p-dopant, the hole transport region 12 may have a matrix (for example, at least one of compounds represented by Formulae 201 to 205) and a p-dopant included in the matrix. The p-dopant may be uniformly or non-uniformly doped in the hole transport region 12.

In an embodiment, the LUMO energy level of the p-dopant may be−3.5 eV or less.

The p-dopant may include a quinone derivative, a metal oxide, a cyano group-containing compound, or a combination thereof.

In an embodiment, the p-dopant may include at least one selected from: a quinone derivative, such as tetracyanoquinodimethane (“TCNQ”), 2,3,5,6-tetrafluoro-7,7,8,8-tetracyanoquinodimethane (F4-TCNQ), and F6-TCNNQ;
a metal oxide, such as tungsten oxide or molybdenum oxide;
1,4,5,8,9,12-hexaazatriphenylene-hexacarbonitrile (HAT-CN);
a compound represented by Formula 221; or
a combination thereof:

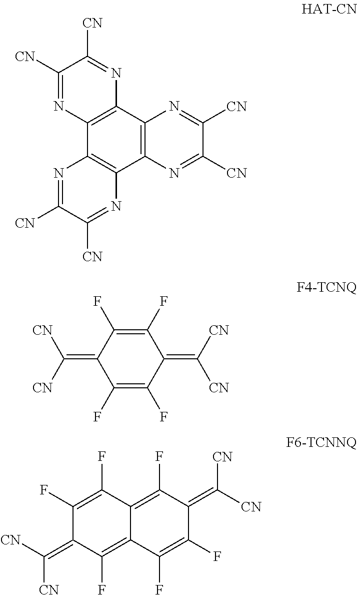

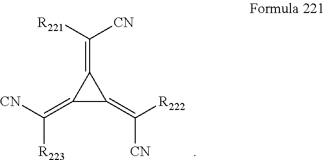

In Formula 221,
R₂₂₁ to R₂₂₃ may each independently be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group, wherein at least one substituent of R₂₂₁ to R₂₂₃ may be: a cyano group; —F; —Cl; —Br; —I; a C₁-C₂₀ alkyl group substituted with —F; a C₁-C₂₀ alkyl group substituted with —Cl; a C₁-C₂₀ alkyl group substituted with —Br; a C₁-C₂₀ alkyl group substituted with —I; or a combination thereof.

The compound represented by Formula 221 may include, for example, Compound HT-D2:

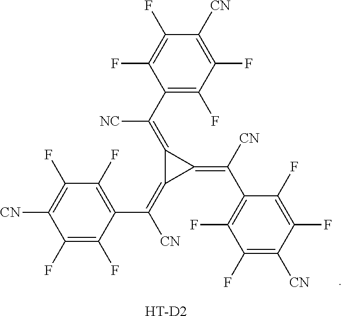

The hole transport region 12 may have a thickness of about 100 Å to about 10000 Å, for example, about 400 Å to about 2000 Å, and the emission layer 15 may have a thickness of about 100 Å to about 3000 Å, for example, about 300 Å to about 1000 Å. When the thickness of each of the hole transport region 12 and the emission layer 15 is within these ranges described above, satisfactory hole transportation characteristics and/or luminescent characteristics may be obtained without a substantial increase in driving voltage.

The hole transport region 12 may further include a buffer layer.

Also, the buffer layer may compensate for an optical resonance distance according to a wavelength of light emitted from the emission layer, and thus, efficiency of a formed organic light-emitting device may be improved.

The hole transport region 12 may further include an electron blocking layer. The electron blocking layer may include a known material, for example, mCP or DBFPO:

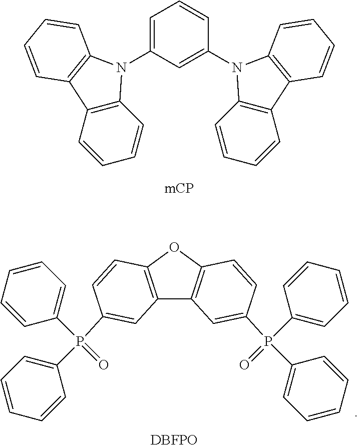

### Electron Transport Region 17

The electron transport region 17 is placed between the emission layer 15 and the second electrode 19 of the organic light-emitting device 10.

The electron transport region 17 may have a single-layered structure or a multi-layered structure.

For example, the electron transport region 17 may have an electron transport layer, an electron transport layer/electron injection layer structure, a buffer layer/electron transport layer structure, hole blocking layer/electron transport layer structure, a buffer layer/electron transport layer/electron injection layer structure, or a hole blocking layer/electron transport layer/electron injection layer structure. The electron transport region 17 may further include an electron control layer.

The electron transport region 17 may include known electron transport materials.

The electron transport region 17 (for example, a buffer layer, a hole blocking layer, an electron control layer, or an electron transport layer in the electron transport region) may include a metal-free compound containing at least one π electron-deficient nitrogen-containing C₁-C₆₀ cyclic group. The π electron-deficient nitrogen-containing C₁-C₆₀ cyclic group is as described above.

For example, the electron transport region 17 may include a compound represented by Formula 601:

[Ar₆₀₁]ₓₑ₁₁-[(L₆₀₁)ₓₑ₁-R₆₀₁]ₓₑ₂₁ Formula 601

wherein, in Formula 601,
Ar₆₀₁ and L₆₀₁ may each independently be a C₅-C₆₀ carbocyclic group unsubstituted or substituted with at least one R₆₀₁ₐ or a C₁-C₆₀ heterocyclic group unsubstituted or substituted with at least one R₆₀₁ₐ,
xe11 may be 1,2, or 3,
xe1 may be an integer from 0 to 5,
R₆₀₁ₐ and R₆₀₁ may each independently be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each independently be a C₁-C₁₀ alkyl group, a C₁-C₁₀ alkoxy group, a phenyl group, a biphenyl group, a terphenyl group, or a naphthyl group, and
xe21 may be an integer from 1 to 5.

In an embodiment, at least one of Ar₆₀₁(s) in the number of xe11 and R₆₀₁(s) in the number of xe21 may include the π electron-deficient nitrogen-containing C₁-C₆₀ cyclic group.

In an embodiment, Ar₆₀₁ and L₆₀₁ in Formula 601 may each independently be a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, a carbazole group, an imidazole group, a pyrazole group, a thiazole group, an isothiazole group, an oxazole group, an isoxazole group, a pyridine group, a pyrazine group, a pyrimidine group, a pyridazine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, or an azacarbazole group, each unsubstituted or substituted with deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, —Si(Q₃₁)(Q₃₂)(Q₃₃), —S(═O)₂(Q₃₁), —P(═O)(Q₃₁)(Q₃₂), or a combination thereof,
Q₃₁ to Q₃₃ may each independently be a C₁-C₁ alkyl group, a C₁-C₁ alkoxy group, a phenyl group, a biphenyl group, a terphenyl group, or a naphthyl group.

When xe11 in Formula 601 is 2 or more, two or more of Ar₆₀₁(s) may be linked to each other via a single bond.

In one or more embodiments, Ar₆₀₁ in Formula 601 may be an anthracene group.

In one or more embodiments, the compound represented by Formula 601 may be represented by Formula 601-1:

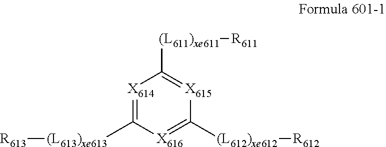

wherein, in Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,
L₆₁₁ to L₆₁₃ may each independently be as described for L₆₀₁,
xe611 to xe613 may each independently be as described for xe1,
R₆₁₁ to R₆₁₃ may each independently be as described for R₆₀₁, and
R₆₁₄ to R₆₁₆ may each independently be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, or a naphthyl group.

In one or more embodiments, xe1 and xe611 to xe613 in Formulae 601 and 601-1 may each independently be 0, 1, or 2.

In one or more embodiments, R₆₀₁ and R₆₁₁ to R₆₁₃ in Formulae 601 and 601-1 may each independently be a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, or an azacarbazolyl group, each unsubstituted or substituted with deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, an azacarbazolyl group, or a combination thereof; or
—S(═O)₂(Q₆₀₁), or —P(═O)(Q₆₀₁)(Q₆₀₂),
wherein Q₆₀₁ and Q₆₀₂ are each as described herein.

The electron transport region 17 may include one of Compounds ET1 to ET36 or a combination thereof:

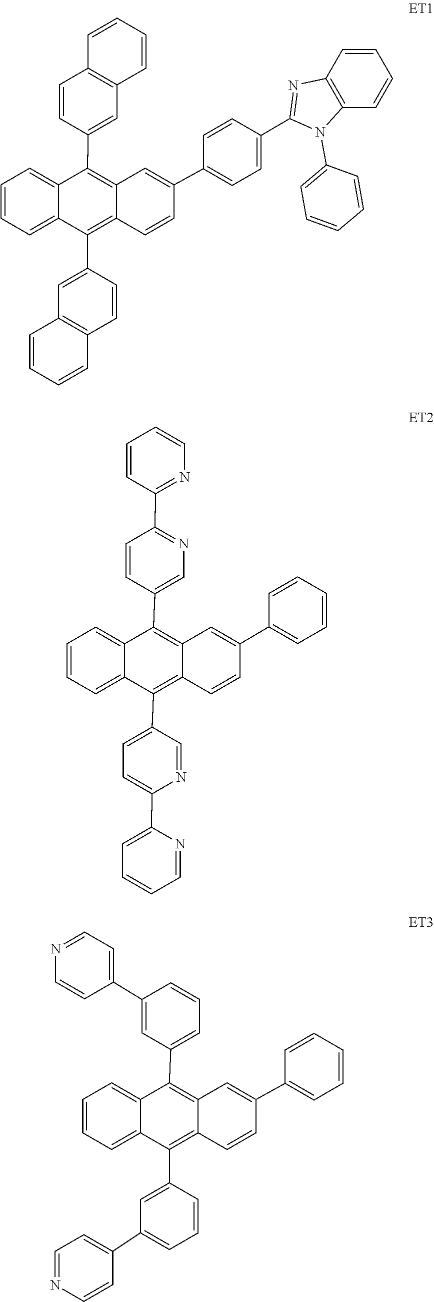 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

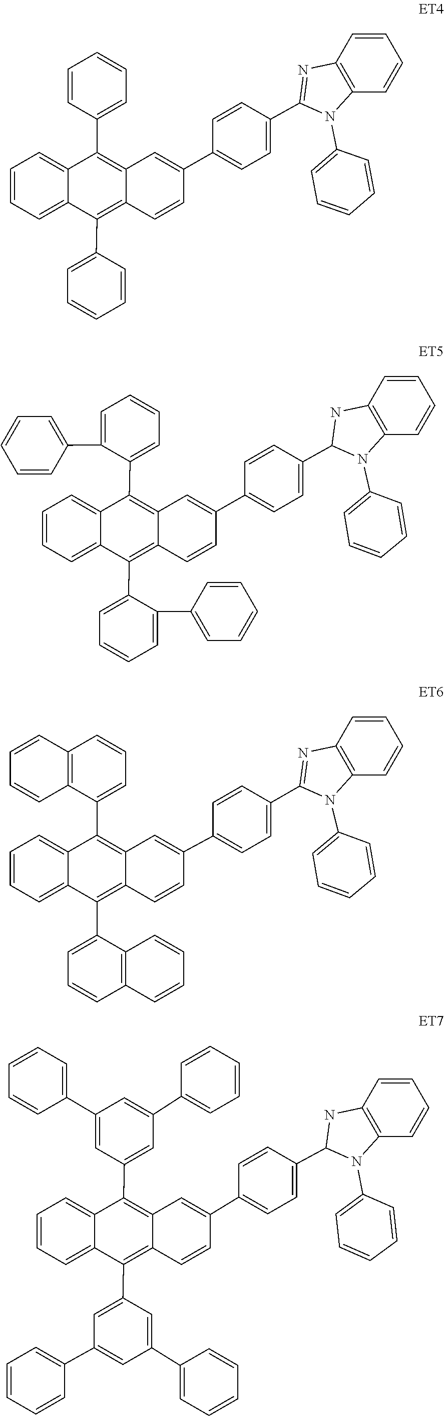

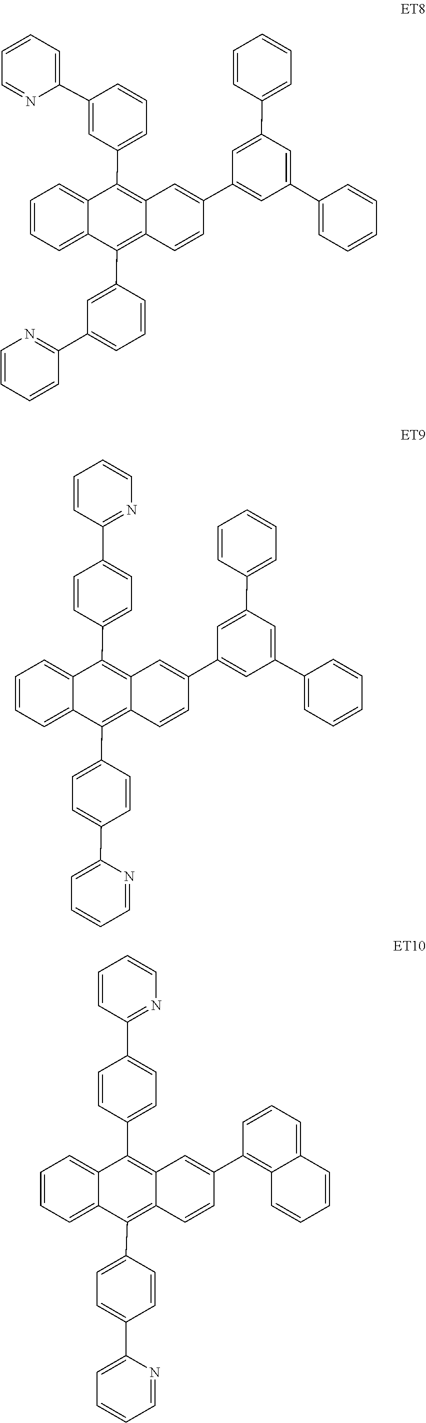

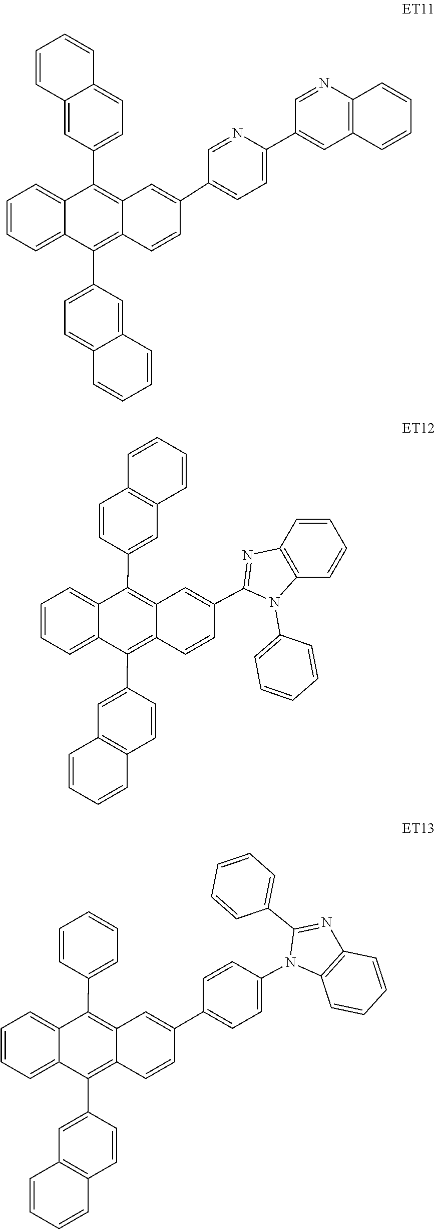 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

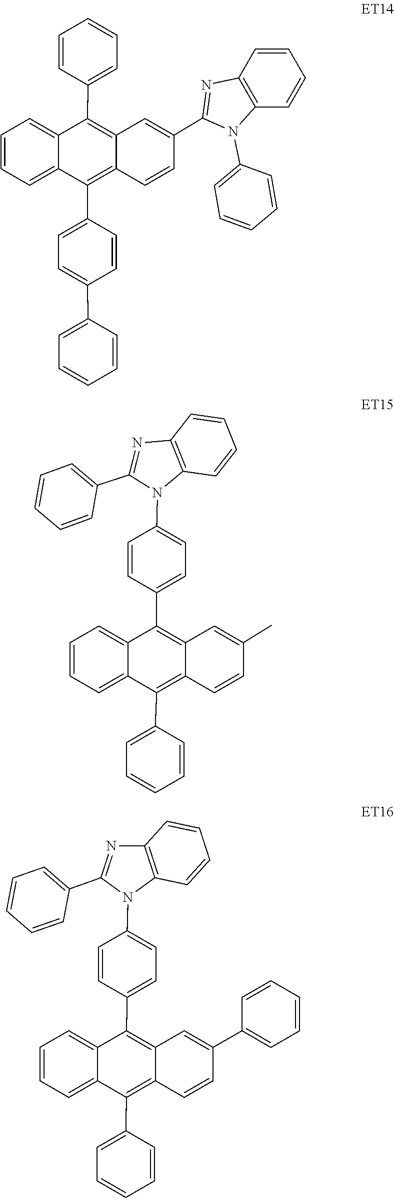

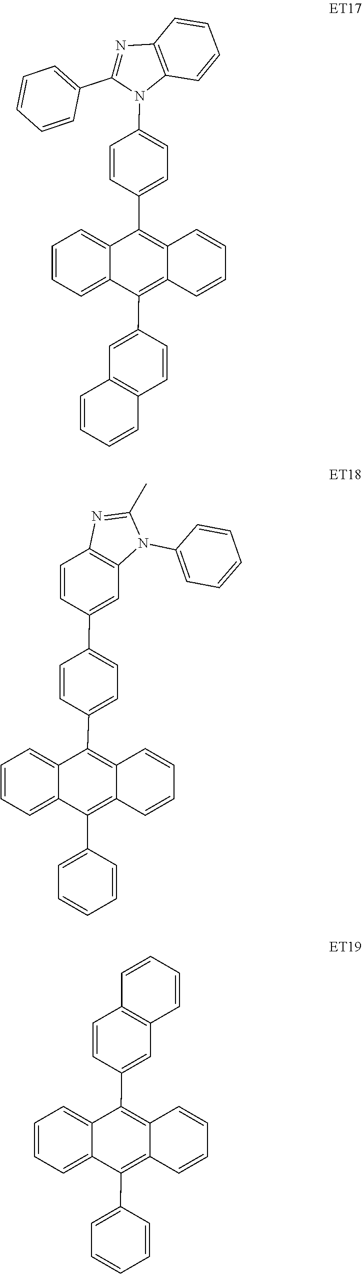

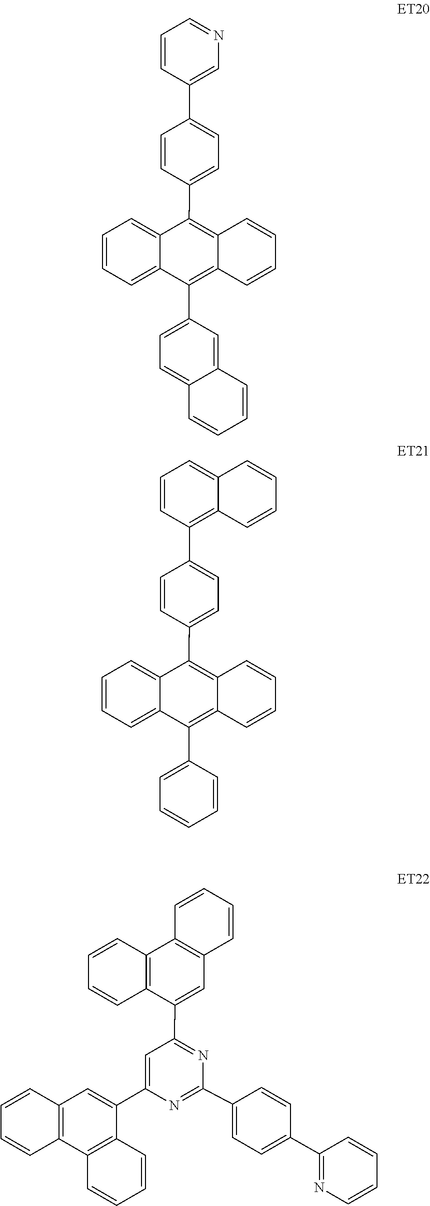

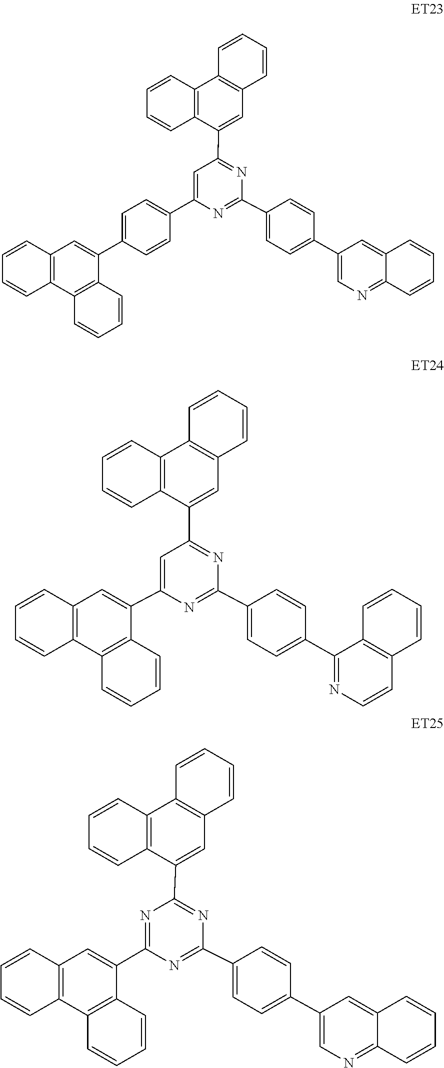 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

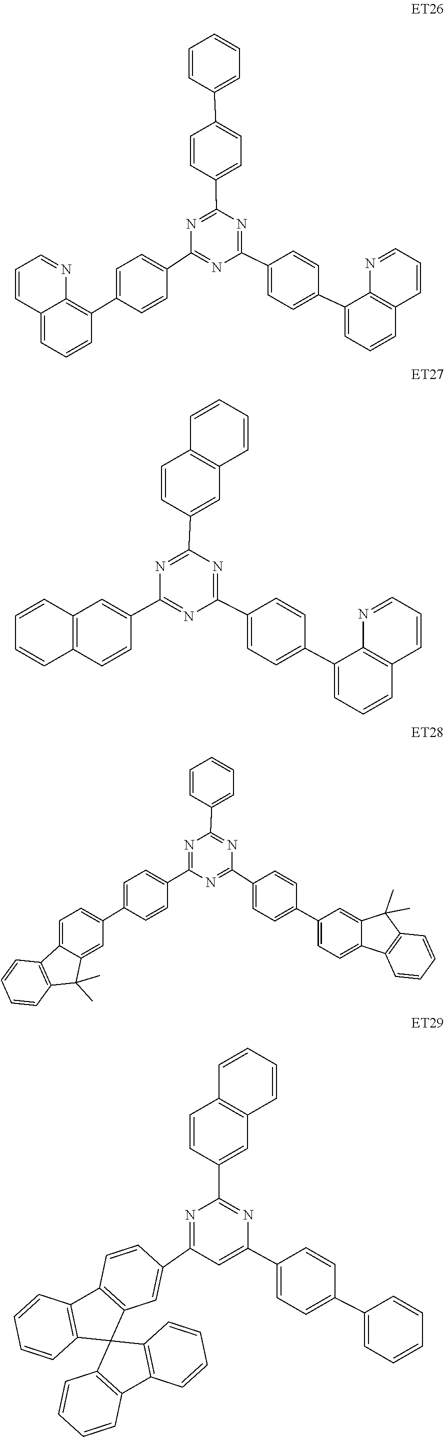 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

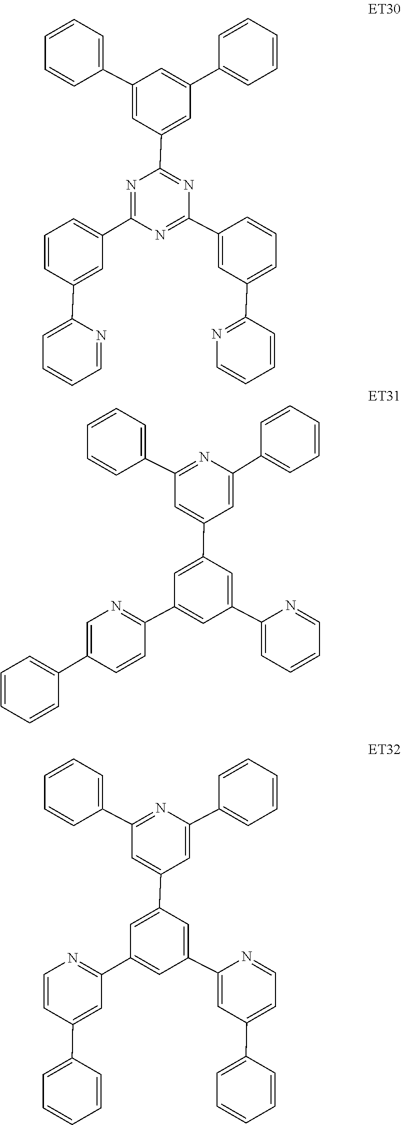

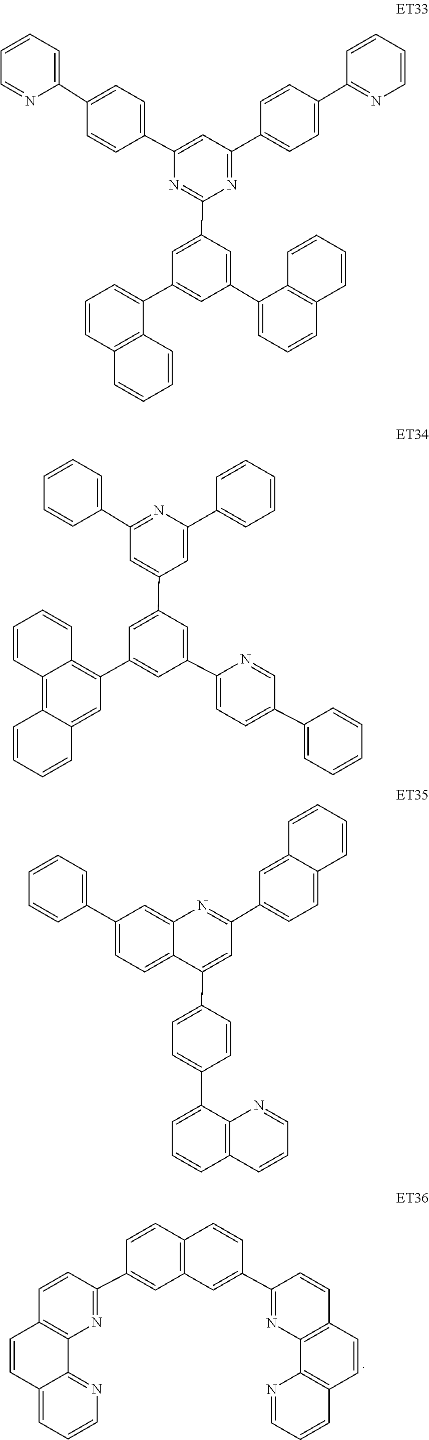

In one or more embodiments, the electron transport region 17 may include 2,9-dimethyl-4,7-diphenyl-1,10-phenanthroline (BCP), 4,7-diphenyl-1,10-phenanthroline (Bphen), Alq3, BAIq, 3-(biphenyl-4-yl)-5-(4-tert-butylphenyl)-4-phenyl-4H-1,2,4-triazole (“TAZ”), NTAZ, DBFPO, or a combination thereof. For example, when the electron transport region 17 includes a hole blocking layer, the hole blocking layer may include BCP or Bphen:

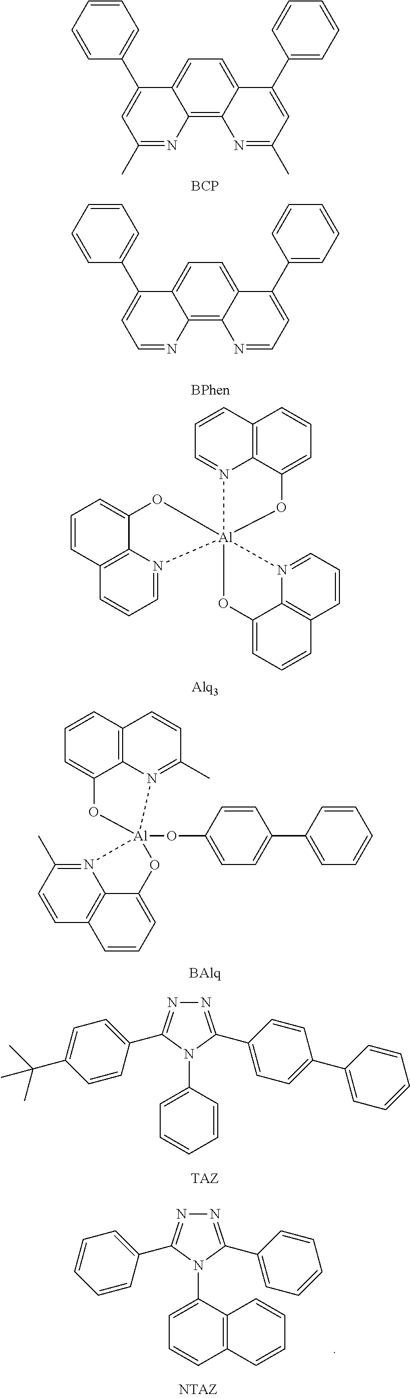

Thicknesses of the buffer layer, the hole blocking layer, and the electron control layer may each independently be in the range of about 20 Å to about 1,000 Å, for example, about 30 Å to about 300 Å. When the thicknesses of the buffer layer, the hole blocking layer, and the electron control layer are within these ranges, excellent hole blocking characteristics or excellent electron control characteristics may be obtained without a substantial increase in driving voltage.

A thickness of the electron transport layer may be in the range of about 100 Å to about 1,000 Å, for example, about 150 Å to about 500 Å. When the thickness of the electron transport layer is within the range described above, the electron transport layer may have satisfactory electron transporting characteristics without a substantial increase in driving voltage.

The electron transport region 17 (for example, the electron transport layer in the electron transport region 17) may further include, in addition to the materials described above, a metal-containing material.

The metal-containing material may include an alkali metal complex, an alkaline earth metal complex, or a combination thereof. A metal ion of the alkali metal complex may include a Li ion, a Na ion, a K ion, a Rb ion, a Cs ion, or a combination thereof, and a metal ion of the alkaline earth metal complex may include a Be ion, a Mg ion, a Ca ion, a Sr ion, a Ba ion, or a combination thereof. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxydiphenyloxadiazole, a hydroxydiphenylthiadiazole, a hydroxyphenylpyridine, a hydroxyphenylbenzimidazole, a hydroxyphenylbenzothiazole, a bipyridine, a phenanthroline, a cyclopentadiene, or a combination thereof.

In an embodiment, the metal-containing material may include a Li complex. The Li complex may include, for example, Compound ET-D1 (LiQ) or ET-D2:

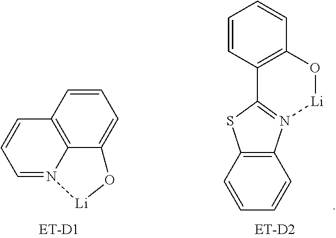

The electron transport region 17 may include an electron injection layer that facilitates the injection of electrons from the second electrode 19. The electron injection layer may directly contact the second electrode 19.

The electron injection layer may have i) a single-layered structure including a single layer including a single material, ii) a single-layered structure including a single layer including a plurality of different materials, or iii) a multi-layered structure having a plurality of layers including a plurality of different materials.

The electron injection layer may include an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or a combination thereof.

The alkali metal may include Li, Na, K, Rb, Cs, or a combination thereof. In an embodiment, the alkali metal may be Li, Na, or Cs. In one or more embodiments, the alkali metal may be Li or Cs.

The alkaline earth metal may include Mg, Ca, Sr, Ba, or a combination thereof.

The rare earth metal may include Sc, Y, Ce, Tb, Yb, Gd, or a combination thereof.

The alkali metal compound, the alkaline earth metal compound, and the rare earth metal compound may include oxides and halides (for example, fluorides, chlorides, bromides, or iodides) of the alkali metal, the alkaline earth metal, and the rare earth metal, or a combination thereof.

The alkali metal compound may include: one of alkali metal oxides such as Li₂O, Cs₂O, or K₂O; one of alkali metal halides such as LiF, NaF, CsF, KF, Lil, Nal, Csl, or KI; or a combination thereof. In an embodiment, the alkali metal compound may include LiF, Li₂O, NaF, Lil, Nal, Csl, KI, or a combination thereof.

The alkaline earth-metal compound may include one of alkaline earth-metal compounds, such as BaO, SrO, CaO, BaₓSr₁₋ₓO (wherein 0<x<1), or BaₓCa₁₋ₓO (wherein 0<x<1), or a combination thereof. In an embodiment, the alkaline earth metal compound may include BaO, SrO, CaO, or a combination thereof.

The rare earth metal compound may include YbF₃, ScF₃, Sc₂O₃, Y₂O₃, Ce₂O₃, GdF₃, TbF₃, or a combination thereof. In an embodiment, the rare earth metal compound may include YbF₃, ScF₃, TbF₃, YbI₃, ScI₃, TbI₃, or a combination thereof.

The alkali metal complex, the alkaline earth metal complex, and the rare earth metal complex may include an ion of an alkali metal, an alkaline earth metal, and a rare earth metal as described above, and a ligand coordinated with a metal ion of the alkali metal complex, the alkaline earth metal complex, or the rare earth metal complex may include hydroxy quinoline, hydroxy isoquinoline, hydroxy benzoquinoline, hydroxy acridine, hydroxy phenanthridine, hydroxy phenyloxazole, hydroxy phenylthiazole, hydroxy diphenyloxadiazole, hydroxy diphenylthiadiazole, hydroxy phenylpyridine, hydroxy phenylbenzimidazole, hydroxy phenylbenzothiazole, bipyridine, phenanthroline, cyclopentadiene, or a combination thereof.

The electron injection layer may consist of an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or a combination thereof, as described above. In one or more embodiments, the electron injection layer may further include an organic material. When the electron injection layer further includes an organic material, an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or a combination thereof may be homogeneously or non-homogeneously dispersed in a matrix including the organic material.

A thickness of the electron injection layer may be about 1 Å to about 100 Å, and, for example, about 3 Å to about 90 Å. When the thickness of the electron injection layer is within the ranges described above, satisfactory electron injection characteristics may be obtained without a substantial increase in driving voltage.

### Second Electrode 19

The second electrode 19 may be on the interlayer 10A. The second electrode 19 may be a cathode which is an electron injection electrode, and in this regard, a material for forming the second electrode 19 may be selected from a metal, an alloy, an electrically conductive compound, and a combination thereof, which have a relatively low work function.

The second electrode 19 may include lithium (Li), silver (Ag), magnesium (Mg), aluminum (AI), aluminum-lithium (Al—Li), calcium (Ca), magnesium-indium (Mg—In), magnesium-silver (Mg—Ag), ITO, IZO, or a combination thereof. The second electrode 19 may be a transmissive electrode, a semi-transmissive electrode, or a reflective electrode.

The second electrode 19 may have a single-layered structure having a single layer or a multi-layered structure including two or more layers.

### Explanation of Terms

The term “C₁-C₆₀ alkyl group” as used herein refers to a linear or branched saturated aliphatic hydrocarbons monovalent group having 1 to 60 carbon atoms, and the term “C₁-C₆₀ alkylene group, as used here refers to a divalent group having the same structure as the C₁-C₆₀ alkyl group.

Examples of the C₁-C₆₀ alkyl group, the C₁-C₂₀ alkyl group, and/or the C₁-C₁₀ alkyl group are a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, or a tert-decyl group, each unsubstituted or substituted with a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, or a combination thereof.

The term “C₁-C₆₀ alkoxy group” used herein refers to a monovalent group represented by —OA₁₀₁ (wherein A₁₀₁ is the C₁-C₆₀ alkyl group), and examples thereof are a methoxy group, an ethoxy group, a propoxy group, a butoxy group, and a pentoxy group.

The term “C₂-C₆₀ alkenyl group” as used herein refers to a hydrocarbon group formed by substituting at least one carbon-carbon double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group formed by substituting at least one carbon-carbon triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon cyclic group having 3 to 10 carbon atoms, and the term “C₃-C₁₀ cycloalkylene group” as used herein refers to a divalent group having the same structure as the C₃-C₁₀ cycloalkyl group.

Examples of the C₃-C₁₀ cycloalkyl group may include a cyclopropyl group, a cyclobutyl group, a cyclopentyl, cyclohexyl group, a cycloheptyl group, a cyclooctyl group, an adamantanyl group, a norbornanyl group (a bicyclo[2.2.1]heptyl group), a bicyclo[1.1.1]pentyl group, a bicyclo[2.1.1]hexyl group, and a bicyclo[2.2.2]octyl group.

The term “C₁-C₁₀ heterocycloalkyl group” as used herein refers to a monovalent monocyclic group that includes at least one heteroatom selected from N, O, P, Si, S, Se, Ge, and B as a ring-forming atom and 1 to 10 carbon atoms, and the term “the C₁-C₁₀ heterocycloalkylene group” as used herein refers to a divalent group having the same structure as the C₁-C₁ heterocycloalkyl group.

Examples of the C₁-C₁ heterocycloalkyl group are a silolanyl group, a silinanyl group, tetrahydrofuranyl group, a tetrahydro-2H-pyranyl group, and a tetrahydrothiophenyl group.

The term “C₃-C₁₀ cycloalkenyl group” as used herein refers to a monovalent cyclic group that includes 3 to 10 carbon atoms and at least one carbon-carbon double bond in the ring thereof and has no aromaticity, and examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₂-C₁₀ heterocycloalkenyl group” as used herein refers to a monovalent monocyclic group that has at least one heteroatom selected from N, O, P, Si, S, Se, Ge, and B as a ring-forming atom, 2 to 10 carbon atoms, and at least one double bond in its ring. Examples of the C₂-C₁₀ heterocycloalkenyl group are a 2,3-dihydrofuranyl group, and a 2,3-dihydrothiophenyl group. The term “C₂-C₁₀ heterocycloalkenylene group” as used herein refers to a divalent group having the same structure as the C₂-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₁-C₆₀ heteroaryl group” as used herein refers to a monovalent group having a heterocyclic aromatic system having 1 to 60 carbon atoms and including at least one heteroatom selected from N, O, P, Si, S, Se, Ge, and B as a ring-forming atom, and the term “C₁-C₆₀ heteroarylene group” as used herein refers to a divalent group having a heterocyclic aromatic system having 1 to 60 carbon atoms and including at least one heteroatom selected from N, O, P, Si, S, Se, Ge, and B as a ring-forming atom. Examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each include two or more rings, the rings may be fused to each other.

The term “C₆-C₆₀ aryloxy group” as used herein indicates —OA₁₀₂ (wherein A₁₀₂ is the C₆-C₆₀ aryl group), and the term “C₆-C₆₀ arylthio group” as used herein indicates —SA₁₀₃ (wherein A₁₀₃ is the C₆-C₆₀ aryl group).

The term “monovalent non-aromatic condensed polycyclic group” used herein refers to a monovalent group in which two or more rings are condensed with each other, only carbon is used as a ring-forming atom (for example, the number of carbon atoms may be 8 to 60) and the whole molecule is a non-aromaticity group. Examples of the monovalent non-aromatic condensed polycyclic group include a fluorenyl group. The term “divalent non-aromatic condensed polycyclic group” as used herein refers to a divalent group having the same structure as the monovalent non-aromatic condensed polycyclic group described above.

The term “monovalent non-aromatic condensed heteropolycyclic group” as used herein refers to a monovalent group having two or more rings condensed with each other, a heteroatom selected from N, O, P, Si, S, Se, Ge, and B, other than carbon atoms (for example, having 1 to 60 carbon atoms), as a ring-forming atom, and no aromaticity in its entire molecular structure. Examples of the monovalent non-aromatic condensed heteropolycyclic group include a carbazolyl group. The term “divalent non-aromatic condensed heteropolycyclic group” as used herein refers to a divalent group having the same structure as the monovalent non-aromatic condensed heteropolycyclic group described above.

The term “π electron-depleted nitrogen-containing C₁-C₆₀ cyclic group” as used herein refers to a cyclic group having 1 to 60 carbon atoms and including at least one *—N═*′ (wherein * and *′ each indicate a binding site to a neighboring atom) as a ring-forming moiety. For example, the π electron-depleted nitrogen-containing C₁-C₆₀ cyclic group may be a) a first ring, b) a condensed ring in which at least two first rings are condensed, or c) a condensed ring in which at least one first ring and at least one second ring are condensed.

The term “π electron-rich C₃-C₆₀ cyclic group” as used herein refers to a cyclic group having 3 to 60 carbon atoms and not including at least one *—N═*′ (wherein * and *′ each indicate a binding site to a neighboring atom) as a ring-forming moiety. For example, the π electron-rich C₃-C₆₀ cyclic group may be a) a second ring or b) a condensed ring in which at least two second rings are condensed.

The term “C₅-C₆₀ cyclic group” as used herein refers to a monocyclic or polycyclic group having 5 to 60 carbon atoms, and may be, for example, a) a third ring or b) a condensed ring in which two or more third rings are condensed with each other.

The term “C₁-C₆₀ heterocyclic group” as used herein refers to a monocyclic or polycyclic group that has 1 to 60 carbon atoms and includes at least one heteroatom, and may be, for example, a) a fourth ring, b) a condensed ring in which two or more fourth rings are condensed with each other, or c) a condensed ring in which at least one third ring is condensed with at least one fourth ring.

The “first ring” as used herein may be an imidazole group, a pyrazole group, a thiazole group, an isothiazole group, an oxazole group, an isoxazole group, a pyridine group, a pyrazine group, a pyridazine group, a pyrimidine group, a triazole group, a tetrazole group, an oxadiazole group, a triazine group, or a thiadiazole group.

The “second ring” as used herein may be a benzene group, a cyclopentadiene group, a pyrrole group, a furan group, a thiophene group, or a silole group.

The “third ring” as used herein may be a cyclopentane group, a cyclopentadiene group, an indene group, an adamantane group, a norbornene group, a bicyclo[1.1.1]pentane group, a bicyclo[2.1.1]hexane group, a bicyclo[2.2.1]heptane group (a norbornane group), a bicyclo[2.2.2]octane group, a cyclohexane group, a cyclohexene group, or a benzene group.

The “fourth ring” as used herein may be a furan group, a thiophene group, a pyrrole group, a silole group, an oxazole group, an isoxazole group, an oxadiazole group, an isoxadiazole group, an oxatriazole group, an isoxatriazole group, a thiazole group, an isothiazole group, a thiadiazole group, an isothiadiazole group, a thiatriazole group, an isothiatriazole group, a pyrazole group, an imidazole group, a triazole group, a tetrazole group, an azasilole group, a diazasilole group, a triazasilole group, a pyridine group, a pyrimidine group, a pyrazine group, a pyridazine group, or a triazine group.

In some embodiments, the π electron-depleted nitrogen-containing C₁-C₆₀ cyclic group may be an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a benzoisoquinoline group, a phthalazine group, a naphthyridine group, a quinoxaline group, a benzoquinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, an azacarbazole group, an azadibenzofuran group, an azadibenzothiophene group, an azadibenzosilole group, an acridine group, or a pyridopyrazine group.

In some embodiments, the π electron-rich C₃-C₆₀ cyclic group may be a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, a furan group, a thiophene group, an isoindole group, an indole group, a benzofuran group, a benzothiophene group, a benzosilole group, a naphthopyrrole group, a naphthofuran group, a naphthothiophene group, a naphthosilole group, a benzocarbazole group, a dibenzocarbazole group, a dibenzofuran group, a dibenzothiophene group, a carbazole group, a dibenzosilole group, an indenocarbazole group, an indolocarbazole group, a benzofurocarbazole group, a benzothienocarbazole group, a benzosilolocarbazole group, a triindolobenzene group, a pyrrolophenanthrene group, a furanophenanthrene group, a thienophenanthrene group, a benzonaphthofuran group, a benzonapthothiophene group, an (indolo)phenanthrene group, a (benzofurano)phenanthrene group, or a (benzothieno)phenanthrene group.

For example, the C₅-C₆₀ carbocyclic group may be a cyclopentane group, a cyclohexane group, a cyclohexene group, a benzene group, a naphthalene group, an anthracene group, a phenanthrene group, a triphenylene group, a pyrene group, a chrysene group, a 1,2,3,4-tetrahydronaphthalene group, a cyclopentadiene group, an indene group, a fluorene group, an adamantane group, a norbornane group, or a norbornene group.

For example, the C₁-C₆₀ heterocyclic group may be a thiophene group, a furan group, a pyrrole group, a cyclopentadiene group, a silole group, a borole group, a phosphole group, a selenophene group, a germole group, a benzothiophene group, a benzofuran group, an indole group, a benzosilole group, a benzoborole group, a benzophosphole group, a benzoselenophene group, a benzogermole group, a dibenzothiophene group, a dibenzofuran group, a carbazole group, a dibenzosilole group, a dibenzoborole group, a dibenzophosphole group, a dibenzoselenophene group, a dibenzogermole group, a dibenzothiophene 5-oxide group, a 9H-fluorene-9-one group, a dibenzothiophene 5,5-dioxide group, an azabenzothiophene group, an azabenzofuran group, an azaindole group, an azaindene group, an azabenzosilole group, an azabenzoborole group, an azabenzophosphole group, an azabenzoselenophene group, an azabenzogermole group, an azadibenzothiophene group, an azadibenzofuran group, an azacarbazole group, an azafluorene group, an azadibenzosilole group, an azadibenzoborole group, an azadibenzophosphole group, an azadibenzoselenophene group, an azadibenzogermole group, an azadibenzothiophene 5-oxide group, an aza-9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, or a benzothiadiazole group.

The term “a π electron-deficient nitrogen-containing C₁-C₆₀ cyclic group, a rr electron-rich C₃-C₆₀ cyclic group, a C₅-C₆₀ cyclic group, and a C₁-C₆₀ heterocyclic group” may be part of a condensed cycle or may be a monovalent, a divalent, a trivalent, a tetravalent, a pentavalent, or a hexavalent group, depending on the formula structure.

As used herein, the number of carbons in each group that is substituted (e.g., C₁-C₆₀) excludes the number of carbons in the substituent. For example, a C₁-C₆₀ alkyl group can be substituted with a C₁-C₆₀ alkyl group. The total number of carbons included in the C₁-C₆₀ alkyl group substituted with the C₁-C₆₀ alkyl group is not limited to 60 carbons. In addition, more than one C₁-C₆₀ alkyl substituent may be present on the C₁-C₆₀ alkyl group. This definition is not limited to the C₁-C₆₀ alkyl group and applies to all substituted groups that recite a carbon range.

Substituents of the substituted π electron-deficient nitrogen-containing C₁-C₆₀ cyclic group, the substituted π electron-rich C₃-C₆₀ cyclic group, the substituted C₅-C₆₀ cyclic group, the substituted C₁-C₆₀ heterocyclic group, the substituted C₁-C₆₀ alkylene group, the substituted C₂-C₆₀ alkenylene group, the substituted C₂-C₆₀ alkynylene group, the substituted C₃-C₁₀ cycloalkylene group, the substituted C₁-C₁₀ heterocycloalkylene group, the substituted C₃-C₁₀ cycloalkenylene group, the substituted C₁-C₁₀ heterocycloalkenylene group, the substituted C₆-C₆₀ arylene group, the substituted C₁-C₆₀ heteroarylene group, the substituted divalent non-aromatic condensed polycyclic group, the substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may each independently be:
deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ alkylheteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or a combination thereof;
a C₃-C₁₀ cycloalkyl group, a C₁-C₁ heterocycloalkyl group, a C₃-C₁₀ cycloalkenyl group, a C₁-C₁ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ alkylheteroaryl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁ heterocycloalkyl group, a C₃-C₁₀ cycloalkenyl group, a C₁-C₁ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ alkylheteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉), or a combination thereof;
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉); or
a combination thereof, Q₁ to Q₉, Q₁₁ to Q₁₉, Q₂₁ to Q₂₉, and Q₃₁ to Q₃₉ used herein may each independently be: hydrogen; deuterium; —F; —Cl; —Br; —I; a hydroxyl group; a cyano group; a nitro group; an amidino group; a hydrazine group; a hydrazone group; a carboxylic acid group or a salt thereof; a sulfonic acid group or a salt thereof; a phosphoric acid group or a salt thereof; or a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁ heterocycloalkyl group, a C₃-C₁₀ cycloalkenyl group, a C₁-C₁ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group or a monovalent non-aromatic condensed heteropolycyclic group, unsubstituted or substituted with deuterium, a C₁-C₆₀ alkyl group, a C₆-C₆₀ aryl group, or a combination thereof.

For example, Q₁ to Q₉, Q₁₁ to Q₁₉, Q₂₁ to Q₂₉ and Q₃₁ to Q₃₉ described herein may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, a C₁-C₁ alkyl group, a phenyl group, or a combination thereof.

The term “room temperature” used herein refers to a temperature of about 25° C.

The terms “a biphenyl group, a terphenyl group, and a tetraphenyl group (quaterphenyl)” used herein respectively refer to monovalent groups in which two, three, or four phenyl groups which are linked together via a single bond.

Hereinafter, a compound and an organic light-emitting device according to embodiments are described in detail with reference to Synthesis Examples and Examples.

However, the organic light-emitting device is not limited thereto. The wording “‘B’ was used instead of ‘ A″’ used in describing Synthesis Examples means that an amount of ‘A’ used was identical to an amount of ‘B’ used, in terms of a molar equivalent.

### EXAMPLES

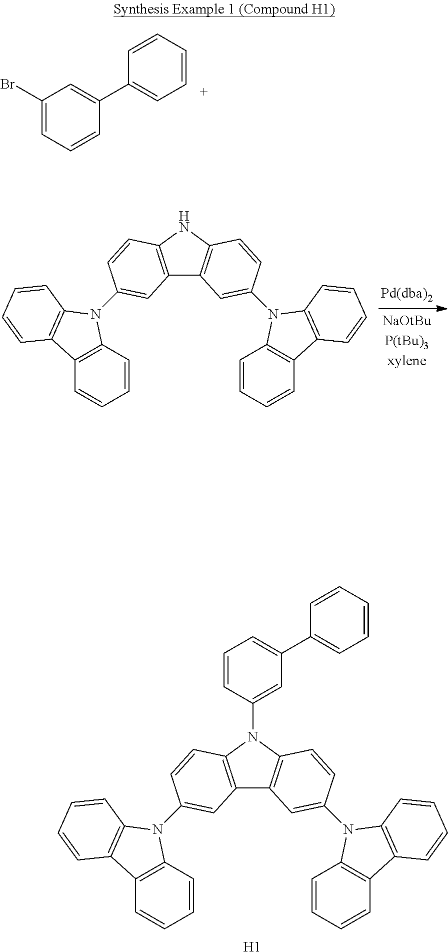 [SMILES: Brc1cccc(-c2ccccc2)c1 ; c1ccc2c(c1)c1ccccc1n2-c1ccc2[nH]c3ccc(-n4c5ccccc5c5ccccc54)cc3c2c1 ; c1ccc(-c2cccc(-n3c4ccc(-n5c6ccccc6c6ccccc65)cc4c4cc(-n5c6ccccc6c6ccccc65)ccc43)c2)cc1 ; "H1"]

3-bromo-1,1′-biphenyl (5.3 g, 23.1 mmol), 9H-9,3′:6′,9″-tercarbazole (12.6 grams (g), 25.4 millimoles (mmol)), Pd(dba)₂ (bis(dibenzylideneacetone)palladium (0)) (1.3 g, 2.3 mmol), sodium tert-butoxide (NaO^{t}Bu) (4.4 g, 46.2 mmol), and tri-tert-butylphosphine (P(tBu)₃) (50 weight percent (wt %) toluene solution, 1.8 g, 4.6 mmol) were mixed with 58 milliliters (ml) of xylene, heated to 115° C., and then stirred for 3 hours. After completion of the reaction, the reaction mixture was allowed to cool to room temperature, the reaction product was added to 800 ml of methanol, stirred for 1 hour, and then filtered under reduced pressure. A solid obtained therefrom was dried and then purified by silica gel column chromatography to provide 2.7 g (yield of 18%) of Compound H1.

LC-Mass (calculated: 649.25 g/mol, found: M⁺¹=650 g/mol).

### Synthesis Example 2 (Compound H4)

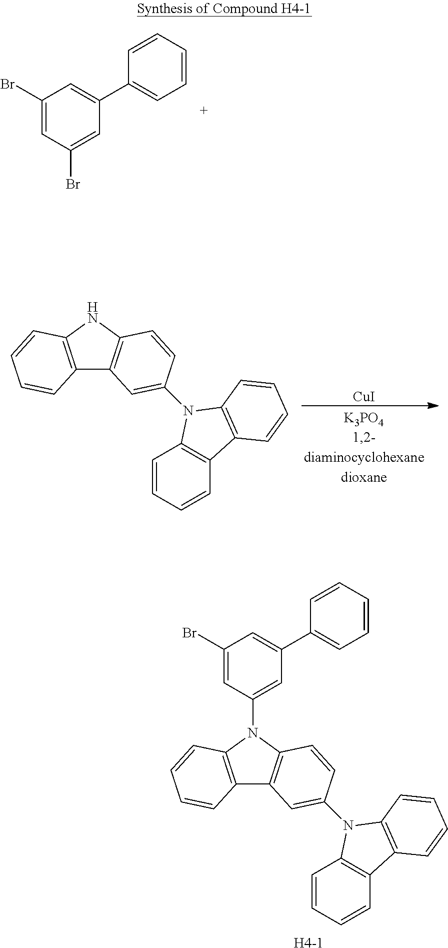

9H-3,9′-bicarbazole (11.5 g, 34.7 mmol), 3,5-dibromo-1,1′-biphenyl (12.99 g, 41.6 mmol), Cul (3.3 g, 17.3 mmol), K₃PO₄ (14.7 g, 69.4 mmol), and 1,2-diaminocyclohexane (4.0 g, 34.7 mmol) were mixed with dioxane (173 ml), heated to 110° C., and then stirred while refluxing for 4 hours. After completion of the reaction, the reaction mixture was allowed to cool to room temperature and an insoluble substance was filtered with a celite pad, subjected to evaporative concentration, and then filtered by silica gel column chromatography to provide 12.4 g (yield of 62%) of Compound H4-1.

LC-Mass (calculated: 562.10 g/mol, found: M⁺¹=563 g/mol).

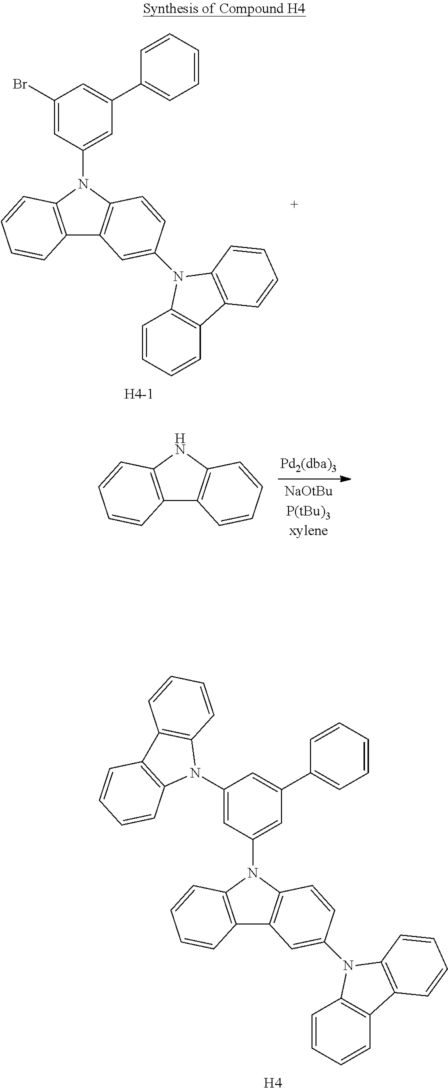

Compound H4-1 (12.4 g, 21.6 mmol), 9H-carbazole (3.8 g, 22.7 mmol), Pd₂(dba)₃ (1.0 g, 1.1 mmol), NaO^{t}Bu (3.1 g, 32.4 mmol), and P(^{t}Bu)₃ (50 wt % toluene solution, 1.5 ml, 3.2 mmol) were mixed with 108 ml of xylene, heated to 130° C., and then stirred for 5 hours. After completion of the reaction, without cooling, the reaction product was filtered with a silica pad, subjected to evaporative concentration, and then purified by silica gel column chromatography to provide 9.7 g (yield of 69%) of Compound H4.

LC-Mass (calculated: 649.25 g/mol, found: M⁺¹=650 g/mol).

### Synthesis Example 3 (Compound H16)

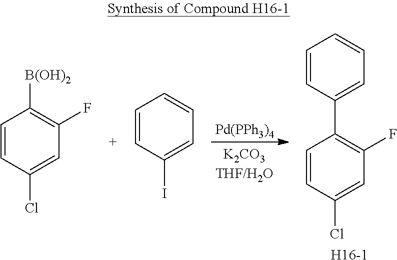

(4-chloro-2-fluorophenyl)boronic acid (10.0 g, 57.4 mmol), iodobenzene (14.0 g, 68.8 mmol), tetrakis(triphenylphosphino)palladium (0) (3.3 g, 2.9 mmol), and K₂CO₃ (23.8 g, 172.1 mmol) were mixed with tetrahydrofuran (THF) (200 ml) and H₂O (90 ml), heated to 80° C., and then stirred while refluxing for 3 hours. After completion of the reaction, the reaction mixture was allowed to cool to room temperature, and an organic layer was extracted with ethyl acetate. A filtrate obtained by filtering the organic layer under reduced pressure after removing water therefrom by using MgSO₄ was subjected to evaporative concentration and then purified with a silica gel column chromatography to provide 11.5 g (yield of 97%) of Compound H16-1.

LC-Mass (calculated: 206.03 g/mol, found M⁺¹=207 g/mol).

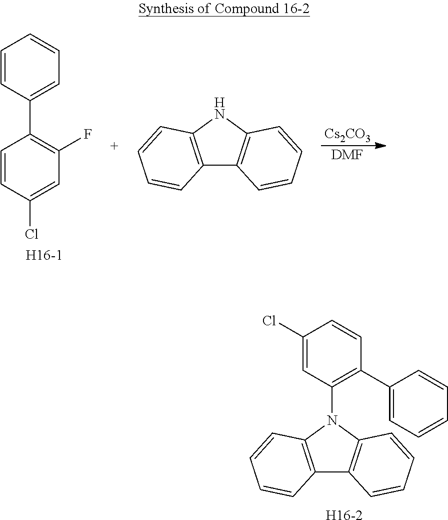

Compound H16-1 (11.0 g, 53.2 mmol), 9H-carbazole (10.7 g, 63.9 mmol), and Cs₂CO₃ (26.0 g, 79.9 mmol) were mixed with 135 ml of N,N-dimethylformamide (DMF), heated to 165° C., and then stirred for 24 hours. After completion of the reaction, the reaction mixture was allowed to cool to room temperature and the reaction product was added to 300 ml of methanol and 400 ml of H₂O, stirred for 1 hour, and then filtered under reduced pressure. A solid obtained therefrom was dried and purified by silica gel column chromatography to provide 15.1 g (yield of 79.9%) of Compound H16-2.

LC-Mass (calculated: 353.10 g/mol, found: M⁺¹=354 g/mol).

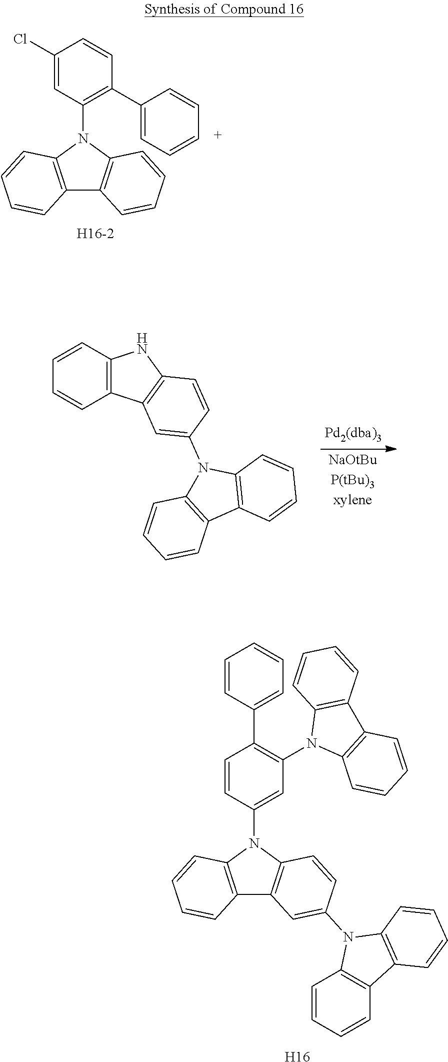

Compound H16-2 (8.0 g, 22.6 mmol), 9H-3,9′-bicarbazole (7.5 g, 22.6 mmol), Pd₂(dba)₃ (2.1 g, 2.3 mmol), NaOtBu (4.3 g, 45.2 mmol), and P(^{t}Bu)₃ (50 wt % toluene solution, 3.7 g, 9.0 mmol) were mixed with 57 ml of xylene, heated to 160° C., and then stirred for 24 hours. After completion of the reaction, without cooling, the reaction product was filtered through a silica pad, subjected to evaporative concentration, and then purified by silica gel column chromatography to provide 9.0 g (yield of 61%) of Compound H16.

LC-Mass (calculated: 649.25 g/mol, found: M⁺¹=650 g/mol).

### Synthesis Example 4 (Compound H27)

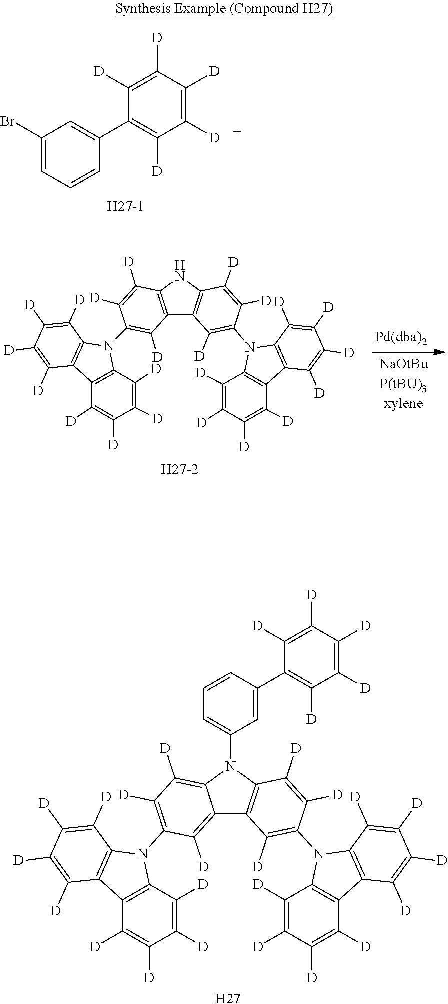

3.11 g (yield of 21%) of Compound H27 was synthesized in the same manner as in Synthesis Example 1, except that Compound H27-1 (3-bromo-1,1′-biphenyl-2′, 3′, 4′, 5′, 6′-d₅) was used instead of 3-bromo-1,1′-biphenyl and Compound H27-2(9H-9,3′: 6′, 9″-tercarbazole-1,1′, 1″, 2,2′, 2″, 3,3″, 4,4′, 4″, 5,5′, 5″, 6,6″, 7,7′, 7″, 8,8′, 8″-d₂₂) was used instead of 9H-9,3′: 6′, 9″-tercarbazole.

LC-Mass (calculated: 676.42 g/mol, found: M⁺¹=677 g/mol).

### Synthesis Example 5 (Compound H36)

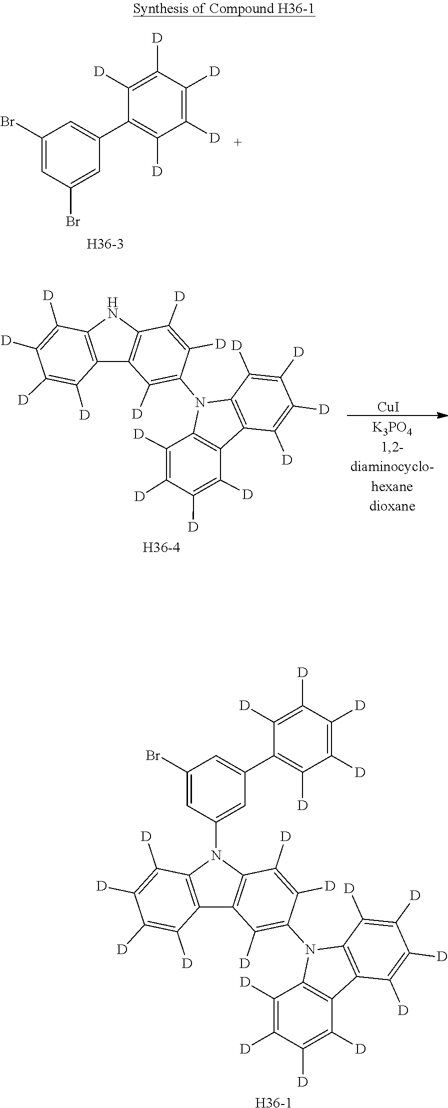

13.20 g (yield of 66%) of Compound H36-1 was synthesized in the same manner as used to synthesize Compound H4-1 of Synthesis Example 2, except that Compound H36-3(3,5-dibromo-1,1′-biphenyl-2′, 3′, 4′, 5′, 6′-d₅) was used instead of 3,5-dibromo-1,1′-biphenyl, and Compound H36-4(9H-3,9′-bicarbazole-1,1′, 2,2′, 3,3′, 4,4′, 5,5′, 6,6′, 7,7′, 8,8′-d₁₅) was used instead of 9H-3,9′-bicarbazole.

LC-Mass (calculated: 582.23 g/mol, found: M⁺¹=583 g/mol).

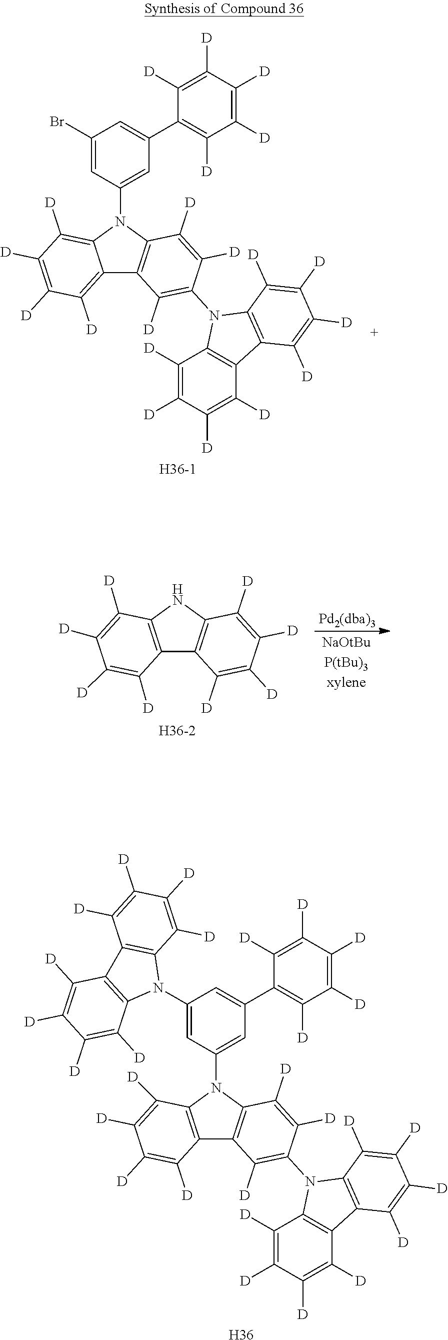

9.32 g (yield of 62%) of Compound H36 was synthesized in the same manner as used to synthesize Compound H4 of Synthesis Example 2, except that Compounds H36-1 and H36-2(9H-carbazole-1,2,3,4,5,6,7,8-d₈) were used instead of Compound H4-1 and 9H-carbazole, respectively.

LC-Mass (calculated: 677.43 g/mol, found: M⁺¹=678 g/mol).

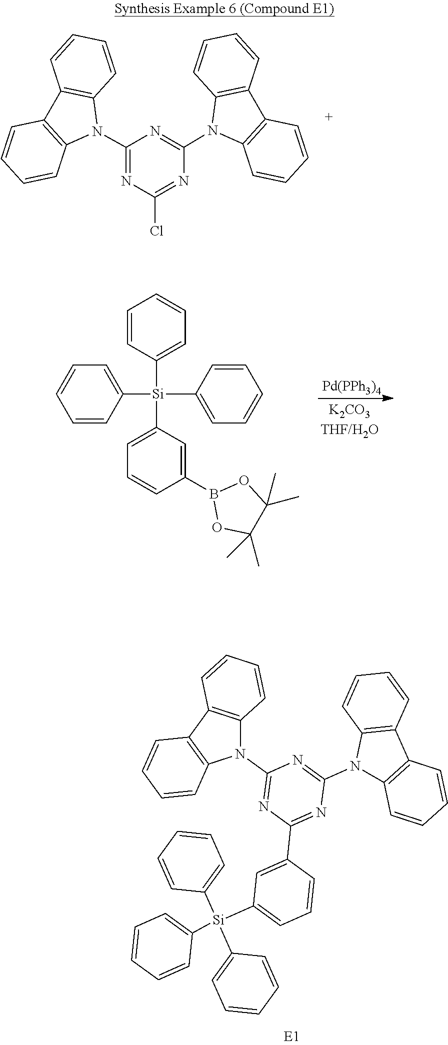 [SMILES: Clc1nc(-n2c3ccccc3c3ccccc32)nc(-n2c3ccccc3c3ccccc32)n1 ; CC1(C)OB(c2cccc([Si](c3ccccc3)(c3ccccc3)c3ccccc3)c2)OC1(C)C ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; "Pd(PPh3)4"]

9,9′-(6-chloro-1,3,5-triazine-2,4-diyl)bis(9H-carbazole) (9.56 g, 21.5 mmol), triphenyl(3-(4,4,5,5-tetramethyl-1,3,2-dioxaborolan-2-yl)phenyl)silane (10.91 g, 23.6 mmol), Pd(PPh₃)₄ (2.48 g, 2.1 mmol), and K₂CO₃ (5.93 g, 42.9 mmol) were mixed with 54 ml of tetrahydrofuran and 22 ml of H₂O, heated to 85° C., and stirred while refluxing for 12 hours. After completion of the reaction, the reaction mixture was allowed to cool to room temperature and then 1,000 ml of methanol was added to the reaction mixture to obtain a solid. Then, the reaction product was purified by silica gel column chromatography to provide 8.5 g (yield of 53%) of Compound E1.

LC-Mass (calculated: 745.27 g/mol, found: M⁺¹=746 g/mol).

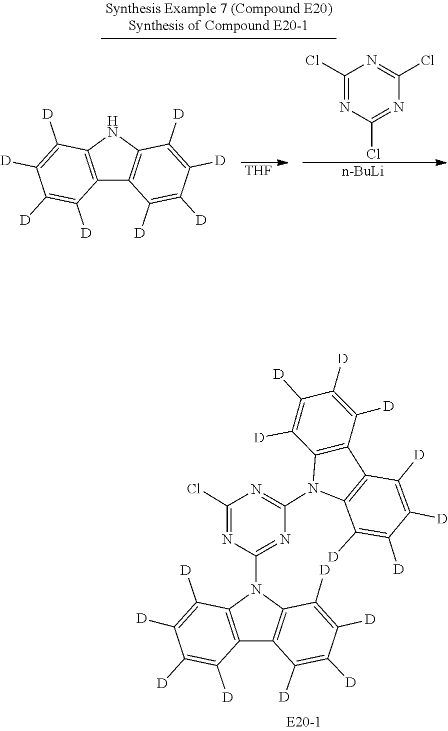

9H-carbazole-1,2,3,4,5,6,7,8-d₈ (8.21 g, 46.7 mmol) and 80 ml of tetrahydrofuran were mixed and then cooled to −78° C. in a nitrogen atmosphere. Then, n-BuLi (2.5M, 24.4 ml) was slowly added dropwise to the mixture at −78° C. and stirred for 1 hour, and then 2,4,6-trichloro-1,3,5-triazine (4.50 g, 24.4 mmol) was further added thereto, and after the reaction mixture was allowed to warm to room temperature, the mixture was stirred for 4 hours. After completion of the reaction, distilled water was slowly added dropwise thereto to terminate the reaction. Then, an organic layer was extracted with ethyl acetate, dried with MgSO₄ and concentrated, and then purified by silica gel column chromatography to provide 8.23 g (yield of 38%) of Compound E20-1.

LC-Mass (calculated: 461.21 g/mol, found: M⁺¹=462 g/mol).

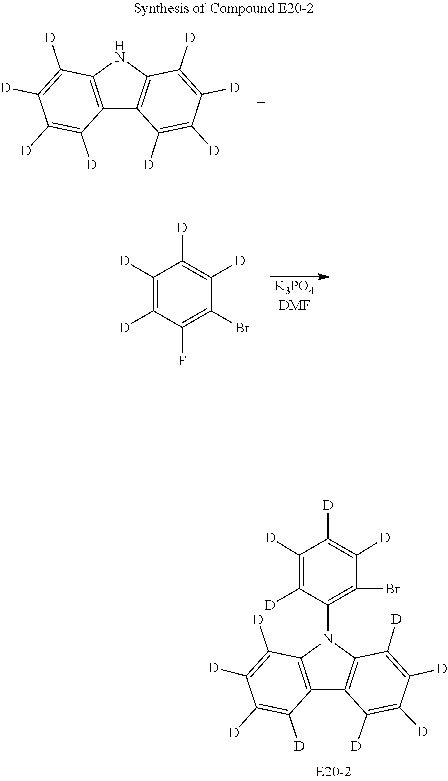

9H-carbazole-1,2,3,4,5,6,7,8-d₈ (12.48 g, 71.2 mmol), 1-bromo-2-fluorobenzene-3,4,5,6-d₄ (10.20 g, 57.0 mmol), and K₃PO₄ (30.53 g, 143.8 mmol) were mixed with 280 ml of N,N-dimethylformamide and then stirred for 20 hours at 165° C. After completion of the reaction, the temperature was cooled to room temperature, and an organic layer was extracted with ethyl acetate, dried with MgSO₄, and concentrated, and then purified by silica gel column chromatography to provide 7.20 g (yield of 30%) of Compound E20-2.

LC-Mass (calculated: 333.09 g/mol, found: M⁺¹=334 g/mol).

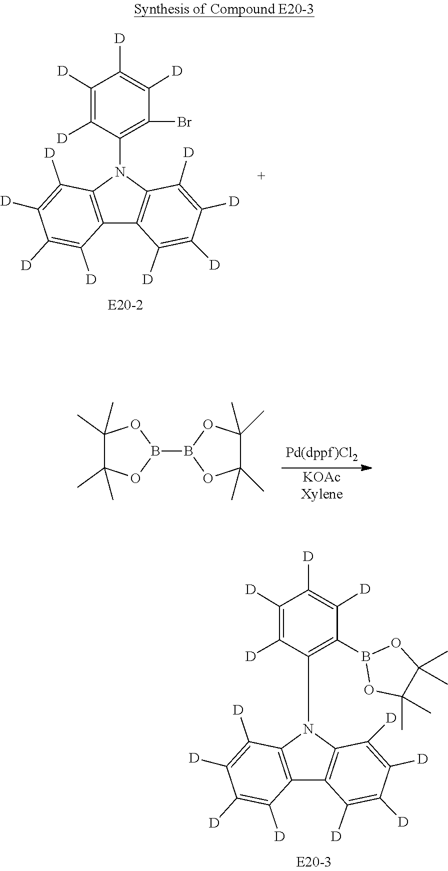

Compound E-2 (13.52 g, 40.4 mmol), bis(pinacolato)diboron (13.91 g, 54.6 mmol) [1,1′-Bis(diphenylphosphino)ferrocene]dichloropalladium (II) (Pd(dppf)Cl₂) (1.71 g, 2.4 mmol), and potassium acetate (KOAc) (10.3 g, 105.1 mmol) were mixed with 160 ml of xylene and then refluxed while heating for 16 hours. Then, after the reaction mixture was allowed to cool to room temperature, the mixture was purified by silica gel column chromatography to provide 6.85 g (yield of 44%) of Compound E20-3.

LC-Mass (calculated: 381.27 g/mol, found: M⁺¹=382 g/mol).

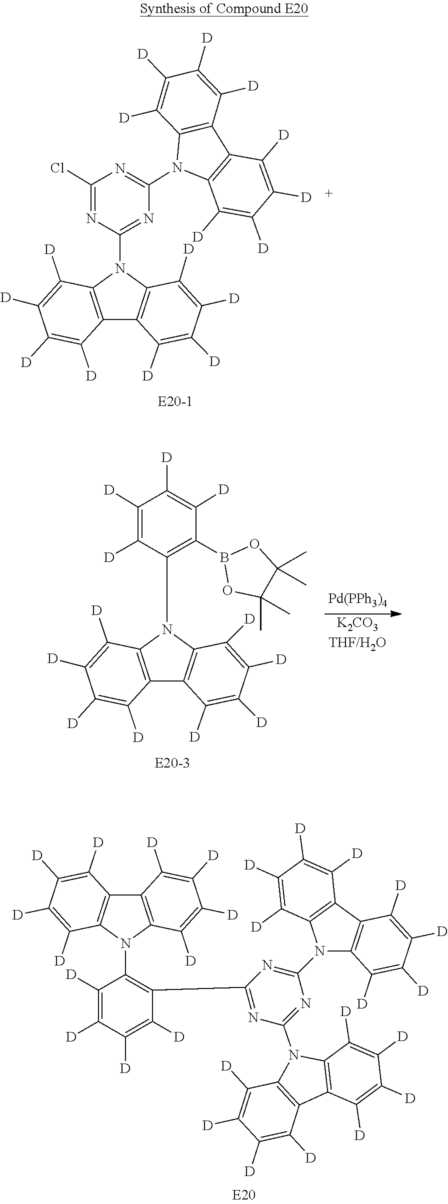

Compound E20-1 (7.12 g, 15.42 mmol), Compound E20-3 (10.14 g, 26.5 mmol), Pd(PPh₃)₄ (1.16 g, 1.0 mmol), and K₂CO₃ (6.55 g, 47.5 mmol) were mixed with 60 ml of tetrahydrofuran and 30 ml of H₂O and then refluxed while heating in a nitrogen atmosphere. The reaction mixture was cooled to room temperature after the mixture was reacted for 18 hours, and an organic layer extracted therefrom with ethyl acetate. The organic layer was dried with MgSO₄ and concentrated under vacuum to provide the crude product which was purified by silica gel column chromatography to provide Compound E20 (9.21 g, yield of 88%).

LC-Mass (calculated: 680.41 g/mol, found: M⁺¹=681 g/mol).

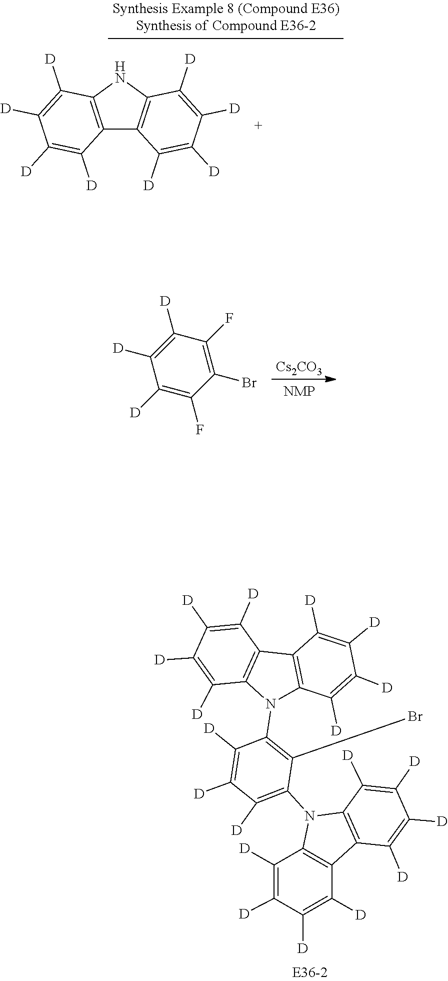

1-bromo-2,6-difluorobenzene-3,4,5-d₃ (5.03 g, 25.7 mmol), 9H-carbazole-1,2,3,4,5,6,7,8-d₈ (9.90 g, 56.5 mmol), and Cs₂CO₃ (20.91 g, 64.2 mmol) were combined with 85 ml of N-methyl-2-pyrrolidone, heated to 180° C., and then stirred for 24 hours. After completion of the reaction, the reaction mixture was cooled to room temperature, and distilled water was added to the reaction mixture to form a precipitate and then the precipitate was filtered. The crude product was purified by silica gel column chromatography to provide 9.9 g (yield of 76%) of Compound E36-2.

LC-Mass (calculated: 505.19 g/mol, found: M⁺¹=502 g/mol).

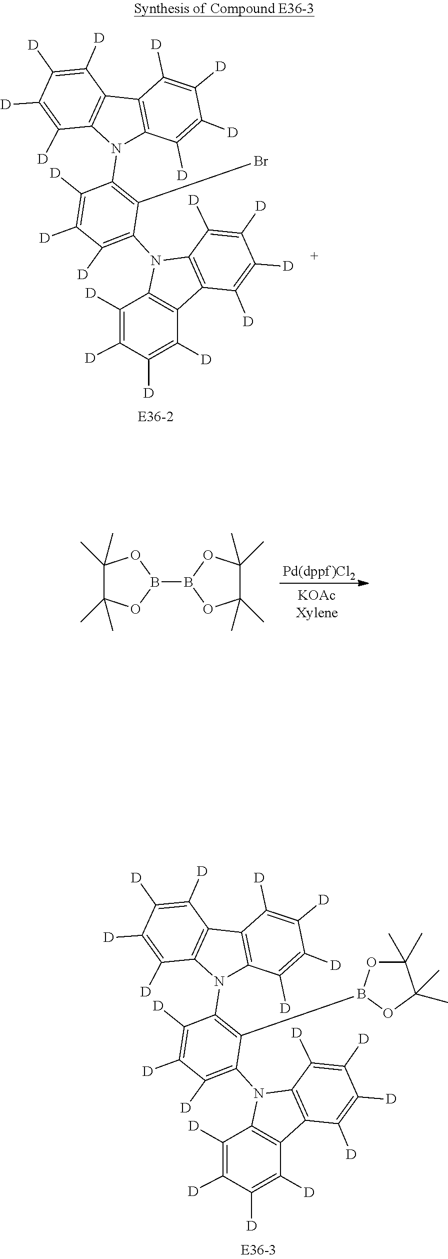

9.5 g of Compound E36-3 (yield of 42%) was provided in the same manner as in synthesizing Compound 20-3 of Synthesis Example 7 except that Compound E36-2 was used instead of Compound E20-2.

LC-Mass (calculated: 553.37 g/mol, found: M⁺¹=554 g/mol).

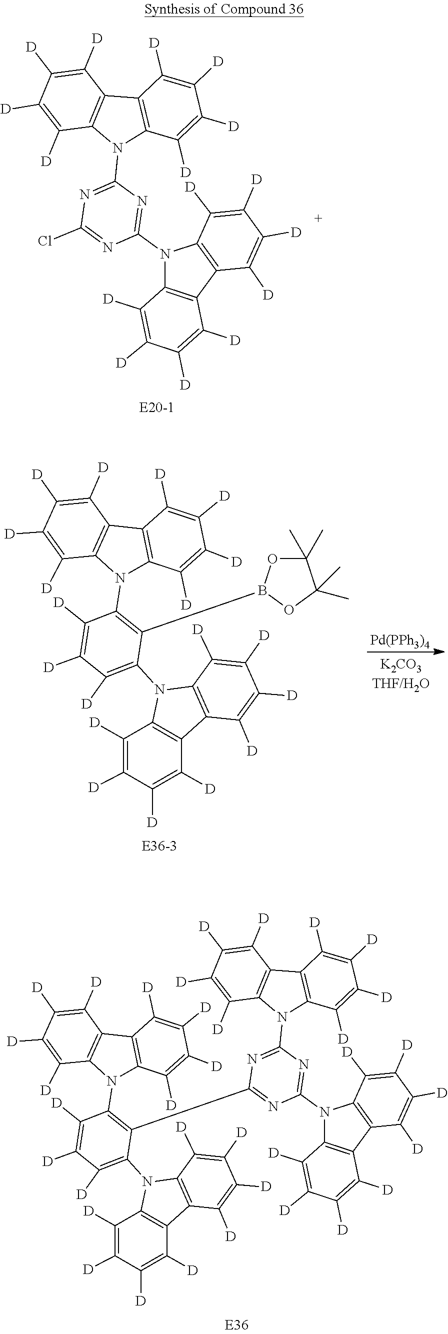

8.7 g of Compound E36 (yield of 79%) was provided in the same manner as in synthesizing Compound E20 of Synthesis Example 7 except that Compound E36-3 was used instead of Compound E20-3.

LC-Mass (calculated: 852.52 g/mol, found: M⁺¹=853 g/mol).

### Evaluation Example 1

T₁ energy level of the compounds of Table 1 were evaluated using the DFT method (T1 adiabatic) of the Gaussian program, which is structure-optimized at the B3LYP/6-31G(d,p) level, and results thereof are shown in Table 1.

TABLE 1

| Compound No. | T₁ energy level (eV) |
|---|---|
| H1 | 2.955 |
| H4 | 3.021 |
| H16 | 2.802 |
| H27 | 2.955 |
| H36 | 3.021 |
| Z1 | 3.023 |
| HA | 2.991 |
| HB | 2.881 |
| HC | 2.951 |
| E1 | 2.994 |
| E20 | 2.619 |
| E36 | 2.701 |
| Z2 | 2.813 |

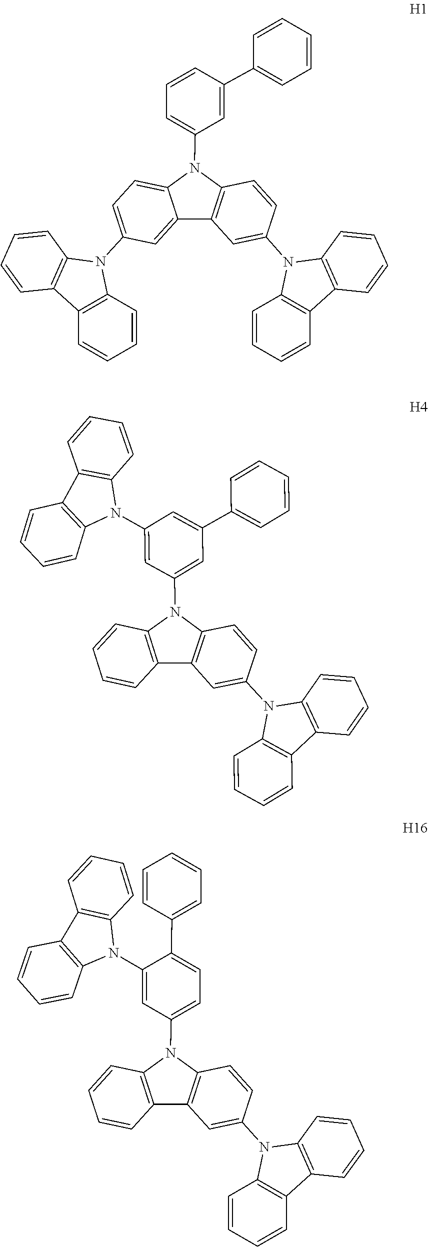 [SMILES: c1ccc(-c2cccc(-n3c4ccc(-n5c6ccccc6c6ccccc65)cc4c4cc(-n5c6ccccc6c6ccccc65)ccc43)c2)cc1 ; c1ccc(-c2cc(-n3c4ccccc4c4ccccc43)cc(-n3c4ccccc4c4cc(-n5c6ccccc6c6ccccc65)ccc43)c2)cc1 ; c1ccc(-c2ccc(-n3c4ccccc4c4cc(-n5c6ccccc6c6ccccc65)ccc43)cc2-n2c3ccccc3c3ccccc32)cc1]

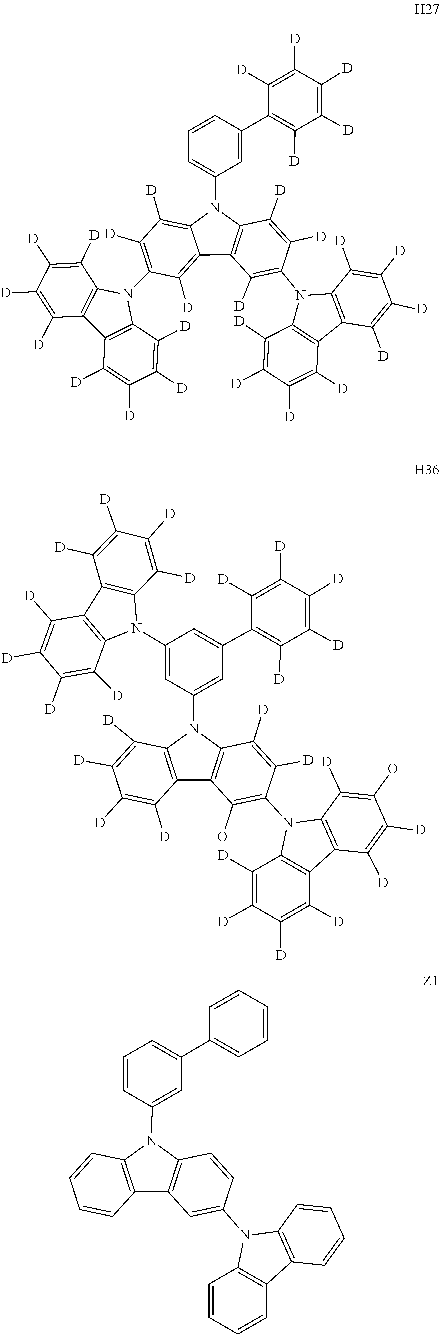

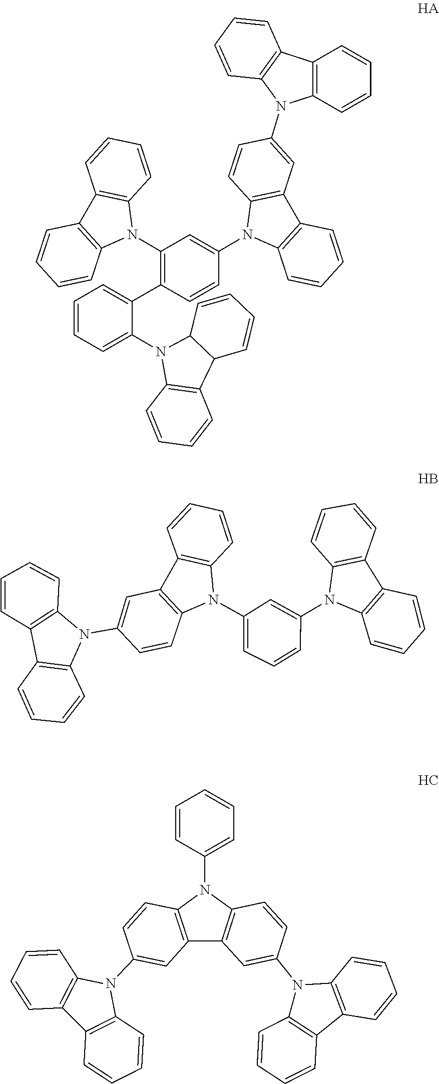 [SMILES: C1=CC2c3ccccc3N(c3ccccc3-c3ccc(-n4c5ccccc5c5cc(-n6c7ccccc7c7ccccc76)ccc54)cc3-n3c4ccccc4c4ccccc43)C2C=C1 ; c1cc(-n2c3ccccc3c3ccccc32)cc(-n2c3ccccc3c3cc(-n4c5ccccc5c5ccccc54)ccc32)c1 ; c1ccc(-n2c3ccc(-n4c5ccccc5c5ccccc54)cc3c3cc(-n4c5ccccc5c5ccccc54)ccc32)cc1]

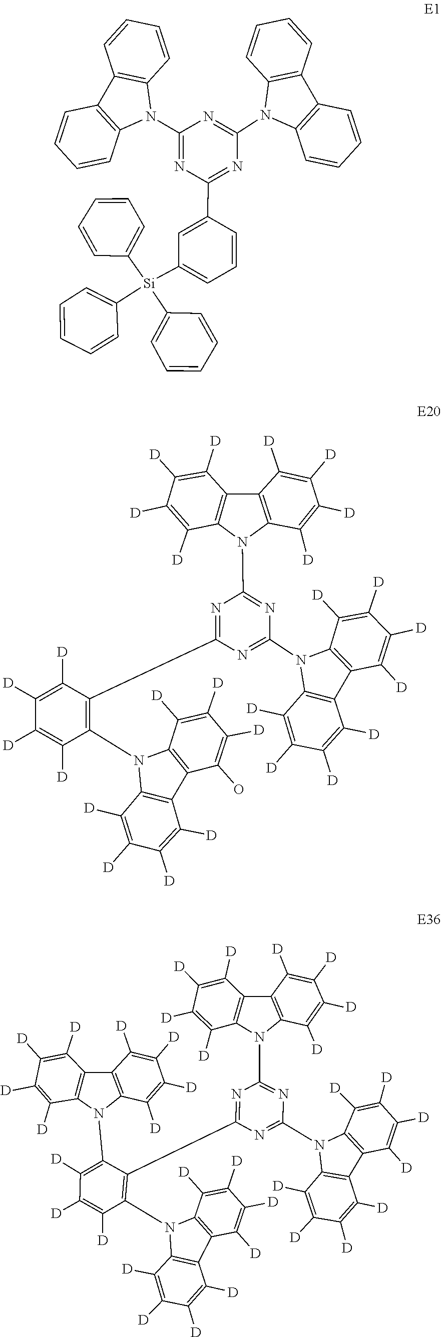

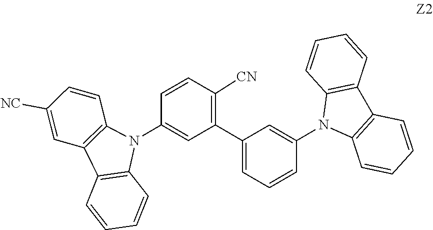 [SMILES: N#Cc1ccc2c(c1)c1ccccc1n2-c1ccc(C#N)c(-c2cccc(-n3c4ccccc4c4ccccc43)c2)c1]

### Example 1-1

A glass substrate with a 1,500 Å-thick indium tin oxide (ITO) electrode (first electrode, anode) formed thereon was cleaned by distilled water ultrasonication. After the distilled water ultrasonication, ultrasonic cleaning was performed with a solvent such as isopropyl alcohol, acetone, and methanol, and the glass substrate was dried and transferred to a plasma cleaner. The glass substrate was cleaned by using oxygen plasma for 5 minutes, and then transferred to a vacuum laminator.

Compounds HT1 and HT-D2 were co-deposited on the ITO electrode of the glass substrate to form a hole injection layer having a thickness of 100 Å. Subsequently, Compound HT1 was deposited on the hole injection layer to form a hole transport layer having a thickness of 1,300 Å. mCP was next deposited on the hole transport layer to form an electron blocking layer having a thickness of 100 Å, thereby forming a hole transport region.

The first compound, the second compound, and the emitter (the weight ratio of the first compound, the second compound, and the emitter is 57:30:13) were co-deposited on the hole transport region to form an emission layer having a thickness of 400 Å.

BCP was vacuum-deposited on the emission layer to form a hole blocking layer having a thickness of 100 Å. Compound ET27 and 8-hydroxyquinolatolithium (“Liq”) were then co-deposited on the hole blocking layer to form an electron transport layer having a thickness of 300 Å. Next, Liq was deposited on the electron transport layer to form an electron injection layer having a thickness of 10 Å, and then, an Al second electrode (a cathode) having a thickness of 1,200 Å was formed on the electron injection layer, thereby completing the manufacture of an organic light-emitting device.

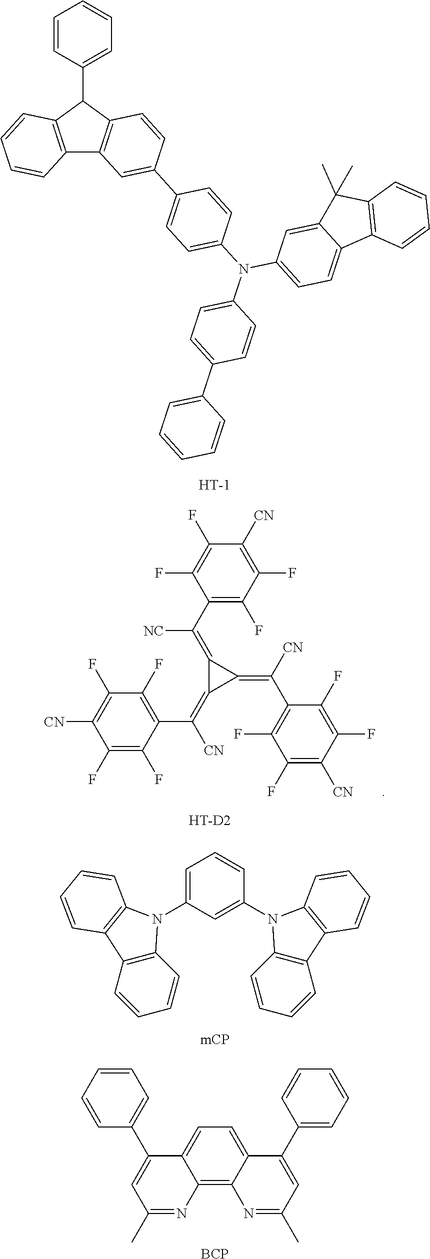

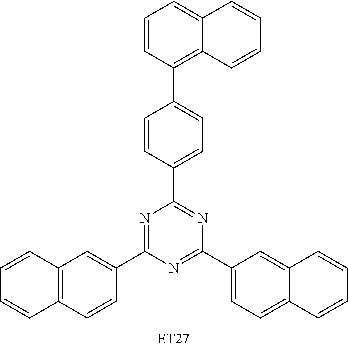 [SMILES: c1ccc2cc(-c3nc(-c4ccc(-c5cccc6ccccc56)cc4)nc(-c4ccc5ccccc5c4)n3)ccc2c1]

### Examples 1-2 to 1-10 and Comparative Examples 1-1 to 1-9

An organic light-emitting device was manufactured in the same manner as in Example 1-1, except that the compounds in Tables 2 to 4 were used when forming the emission layer.

### Example 2-1

An organic light-emitting device was manufactured in the same manner as in Example 1-1 except that the first compound, the second compound, the sensitizer, and the emitter (the weight ratio of the first compound, the second compound, the sensitizer, and the emitter is 56:30.2:13:0.8) of Table 5 were co-deposited on the hole transport region to form an emission layer having a thickness of 400 Å instead of forming the emission layer of Example 1-1.

### Examples 2-2 to 2-10 and Comparative Examples 2-1 to 2-9

An organic light-emitting device was manufactured in the same manner as in Example 2-1, except that the compounds in Tables 5 to 7 were used when forming the emission layer.

### Evaluation Example 2

The lifespan (LT95) of the light-emitting device according to the examples and comparative examples were measured, and the results thereof are shown in Tables 2 to 7. A current-voltage meter (Keithley 2400) and a luminance meter (Minolta Cs-1000A) were used as apparatuses for evaluation, and the lifespan (LT₉₅) (at 1,000 nit) was evaluated by measuring the amount of time (hr) taken for luminance to be reduced to 95% of the initial luminance of 100%. The lifespan of the light-emitting devices of Examples 1-1 to 1-10 and Comparative Examples 1-2 to 1-9 were represented as relative values (%) relative to the lifespan of Comparative Example 1-1, and the lifespan of the light-emitting devices of Examples 2-1 to 2-10 and Comparative Examples 2-2 to 2-9 were represented as relative values (%) relative to the lifespan of Comparative Example 2-1.

TABLE 2

| | Emission layer | | | |
|---|---|---|---|---|
| | Host | | | |
| | First | Second | | LT₉₅ (%) |
| | compound | compound | Emitter | (relative values) |
| Example 1-1 | H1 | E1 | P31 | 203 |
| Example 1-2 | H4 | E1 | P31 | 317 |
| Example 1-3 | H16 | E1 | P31 | 196 |
| Example 1-4 | H27 | E1 | P31 | 300 |
| Example 1-5 | H36 | E1 | P31 | 501 |
| Comparative | Z1 | Z2 | P31 | 100 |
| Example 1-1 | | | | |
| Comparative | HA | E1 | P31 | 166 |
| Example 1-2 | | | | |
| Comparative | HB | E1 | P31 | 173 |
| Example 1-3 | | | | |
| Comparative | HC | E1 | P31 | 124 |
| Example 1-4 | | | | |
| Comparative | Z1 | E1 | P31 | 183 |
| Example 1-5 | | | | |

TABLE 3

| | Emission layer | | | |
|---|---|---|---|---|
| | Host | | | |
| | First | Second | | LT₉₅ (%) |
| | compound | compound | Emitter | (relative values) |
| Example 1-6 | H1 | E20 | P31 | 466 |
| Example 1-7 | H4 | E20 | P31 | 386 |
| Example 1-8 | H16 | E20 | P31 | 333 |
| Comparative | Z1 | Z2 | P31 | 100 |
| Example 1-1 | | | | |
| Comparative | HA | E20 | P31 | 280 |
| Example 1-6 | | | | |
| Comparative | HB | E20 | P31 | 285 |
| Example 1-7 | | | | |
| Comparative | HC | E20 | P31 | 270 |
| Example 1-8 | | | | |
| Comparative | Z1 | E20 | P31 | 320 |
| Example 1-9 | | | | |

TABLE 4

| | Emission layer | | | |
|---|---|---|---|---|
| | Host | | | |
| | First | Second | | LT₉₅ (%) |
| | compound | compound | Emitter | (relative values) |
| Example 1-9 | H1 | E36 | P31 | 226 |
| Example 1-10 | H4 | E36 | P31 | 225 |
| Comparative | Z1 | Z2 | P31 | 100 |
| Example 1-1 | | | | |

TABLE 5

| | Emission layer | | | | |
|---|---|---|---|---|---|
| | Host | | | | LT₉₅ (%) |
| | First | Second | | | (relative |
| | compound | compound | Sensitizer | Emitter | values) |
| Example 2-1 | H1 | E1 | P31 | D3 | 170 |
| Example 2-2 | H4 | E1 | P31 | D3 | 238 |
| Example 2-3 | H16 | E1 | P31 | D3 | 245 |
| Example 2-4 | H27 | E1 | P31 | D3 | 235 |
| Example 2-5 | H36 | E1 | P31 | D3 | 179 |
| Comparative | Z1 | Z2 | P31 | D3 | 100 |
| Example 2-1 | | | | | |
| Comparative | HA | E1 | P31 | D3 | 76 |
| Example 2-2 | | | | | |
| Comparative | HB | E1 | P31 | D3 | 116 |
| Example 2-3 | | | | | |
| Comparative | HC | E1 | P31 | D3 | 94 |
| Example 2-4 | | | | | |
| Comparative | Z1 | E1 | P31 | D3 | 138 |
| Example 2-5 | | | | | |

TABLE 6

| | Emission layer | | | | |
|---|---|---|---|---|---|
| | Host | | | | LT₉₅ (%) |
| | First | Second | | | (relative |
| | compound | compound | Sensitizer | Emitter | values) |
| Example 2-6 | H1 | E20 | P31 | D3 | 338 |
| Example 2-7 | H4 | E20 | P31 | D3 | 380 |
| Example 2-8 | H16 | E20 | P31 | D3 | 367 |
| Comparative | Z1 | Z2 | P31 | D3 | 100 |
| Example 2-1 | | | | | |
| Comparative | HA | E20 | P31 | D3 | 140 |
| Example 2-6 | | | | | |
| Comparative | HB | E20 | P31 | D3 | 202 |
| Example 2-7 | | | | | |
| Comparative | HC | E20 | P31 | D3 | 182 |
| Example 2-8 | | | | | |
| Comparative | Z1 | E20 | P31 | D3 | 248 |
| Example 2-9 | | | | | |

TABLE 7

| | Emission layer | | | | |
|---|---|---|---|---|---|
| | Host | | | | LT₉₅ (%) |
| | First | Second | | | (relative |
| | compound | compound | Sensitizer | Emitter | values) |
| Example 2-9 | H1 | E36 | P31 | D3 | 202 |
| Example 2-10 | H4 | E36 | P31 | D3 | 204 |
| Comparative | Z1 | Z2 | P31 | D3 | 100 |
| Example 2-1 | | | | | |

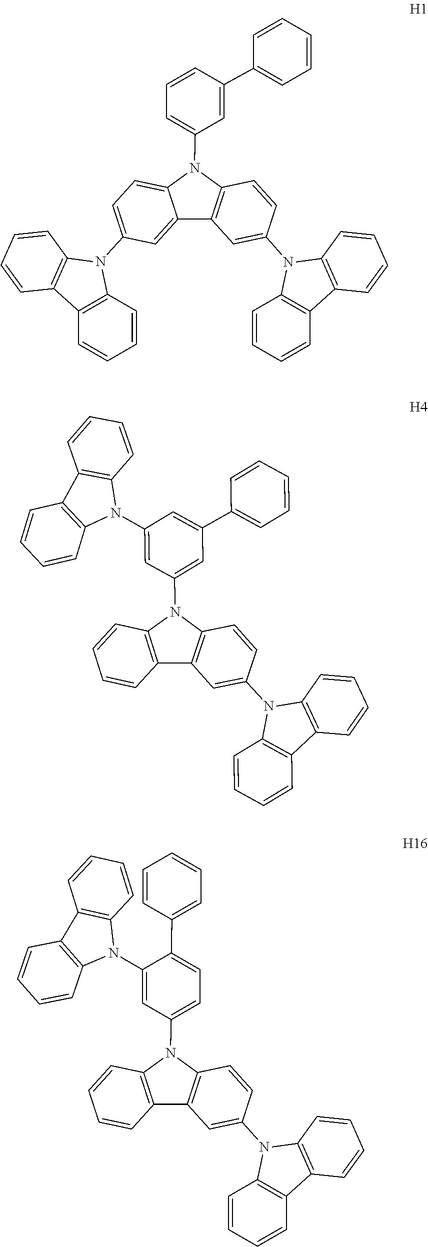 [SMILES: c1ccc(-c2cccc(-n3c4ccc(-n5c6ccccc6c6ccccc65)cc4c4cc(-n5c6ccccc6c6ccccc65)ccc43)c2)cc1 ; c1ccc(-c2cc(-n3c4ccccc4c4ccccc43)cc(-n3c4ccccc4c4cc(-n5c6ccccc6c6ccccc65)ccc43)c2)cc1 ; c1ccc(-c2ccc(-n3c4ccccc4c4cc(-n5c6ccccc6c6ccccc65)ccc43)cc2-n2c3ccccc3c3ccccc32)cc1]

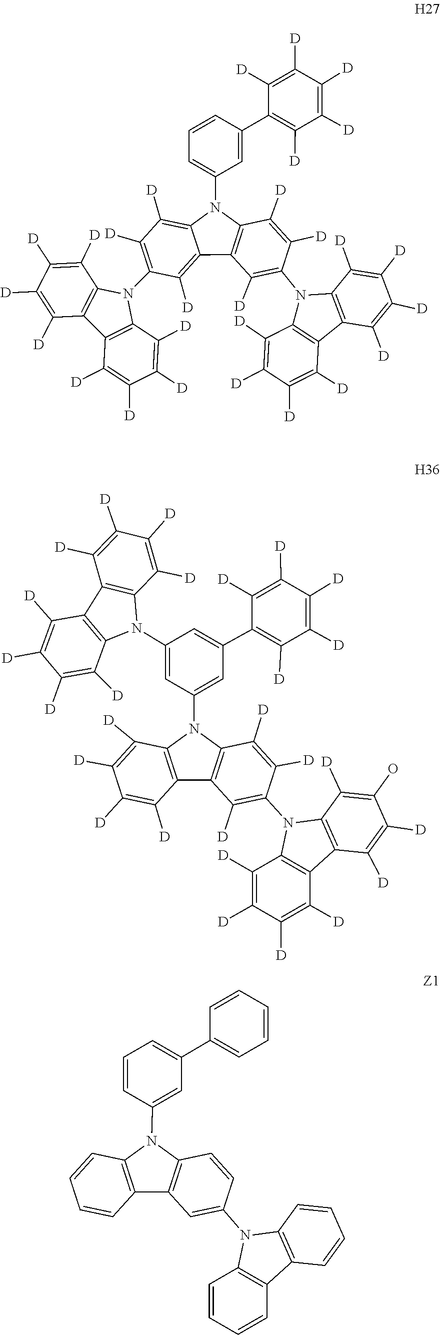

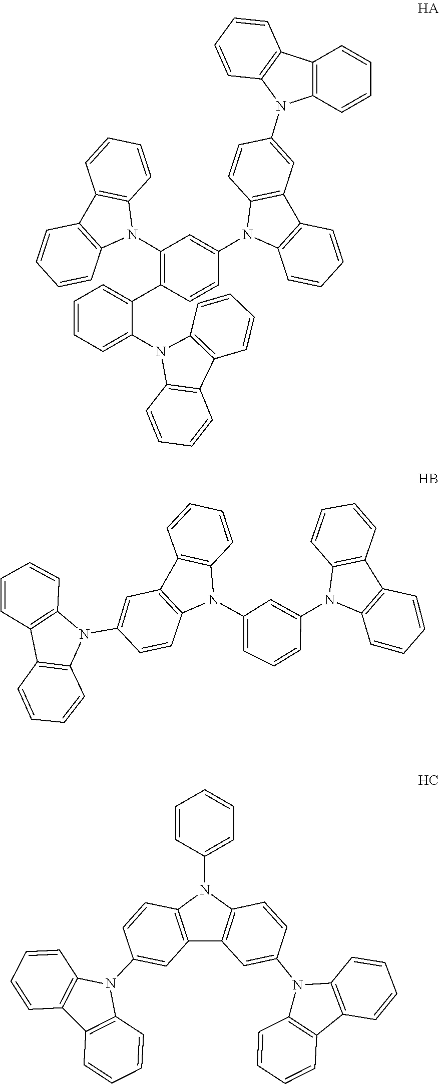 [SMILES: c1ccc(-n2c3ccccc3c3ccccc32)c(-c2ccc(-n3c4ccccc4c4cc(-n5c6ccccc6c6ccccc65)ccc43)cc2-n2c3ccccc3c3ccccc32)c1 ; c1cc(-n2c3ccccc3c3ccccc32)cc(-n2c3ccccc3c3cc(-n4c5ccccc5c5ccccc54)ccc32)c1 ; c1ccc(-n2c3ccc(-n4c5ccccc5c5ccccc54)cc3c3cc(-n4c5ccccc5c5ccccc54)ccc32)cc1]

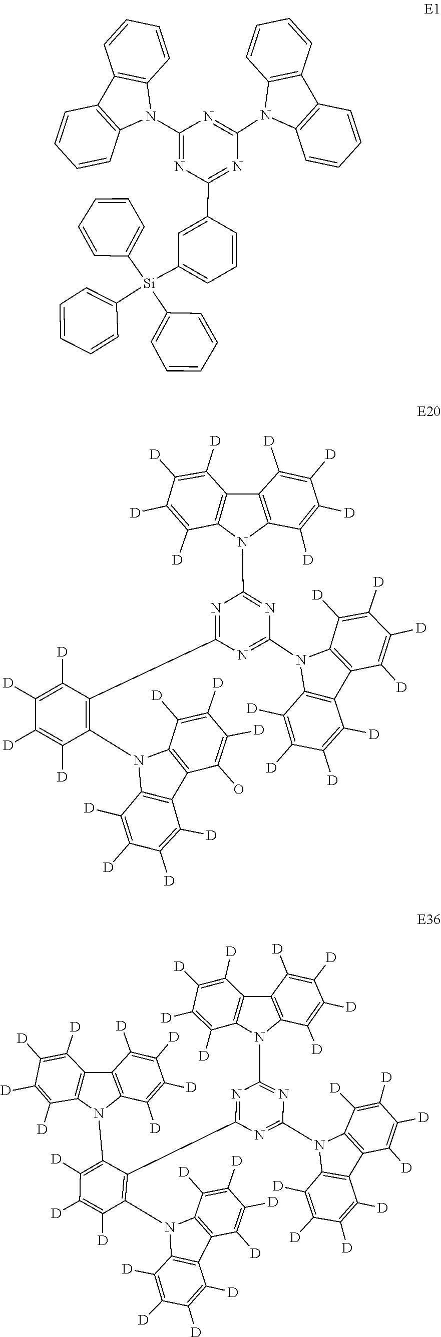

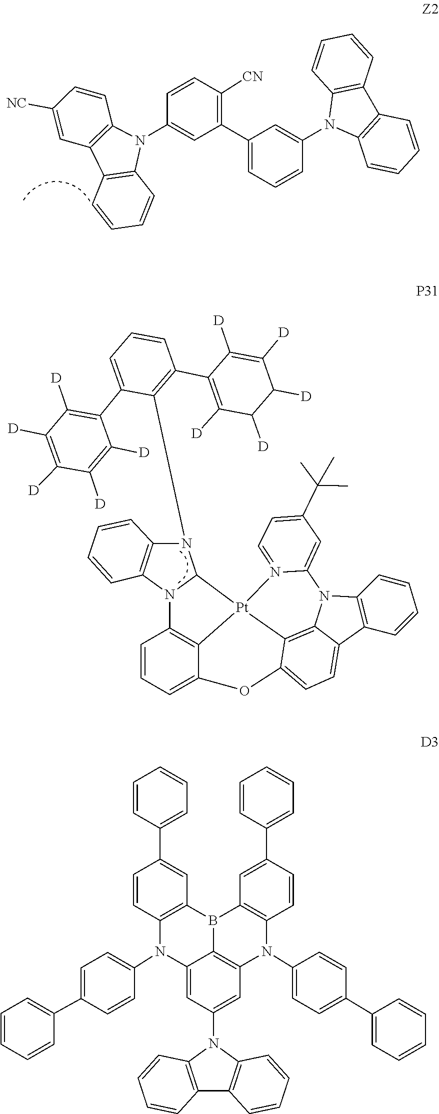

1) From Table 2, the organic light-emitting devices of Examples 1-1 to 1-5 were found to have excellent lifespan characteristics as compared with those of Comparative Examples 1-1 to 1-5,
2) from Table 3, the organic light-emitting devices of Examples 1-6 to 1-8 were found to have excellent lifespan characteristics as compared with those of Comparative Examples 1-1 and 1-6 to 1-9,
3) From Table 4, the organic light-emitting devices of Examples 1-9 and 1-10 were found to have excellent lifespan characteristics as compared with that of Comparative Example 1-1,
4) From Table 5, the organic light-emitting devices of Examples 2-1 to 2-5 were found to have excellent lifespan characteristics as compared with those of Comparative Examples 2-1 to 2-5,
5) from Table 6, the organic light-emitting devices of Examples 2-6 to 2-8 were found to have excellent lifespan characteristics as compared with those of Comparative Examples 2-1 and 2-6 to 2-9, and
6) from Table 7, the organic light-emitting devices of Examples 2-9 and 2-10 were found to have excellent lifespan characteristics as compared with that of Comparative Example 2-1.

According to the one or more embodiments, the light-emitting device including the composition has excellent lifespan characteristics, and thus, a high-quality electronic apparatus using the light-emitting device may be manufactured.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A composition comprising:
a first compound represented by Formula 1; and
a second compound comprising an azine group comprising at least one nitrogen: 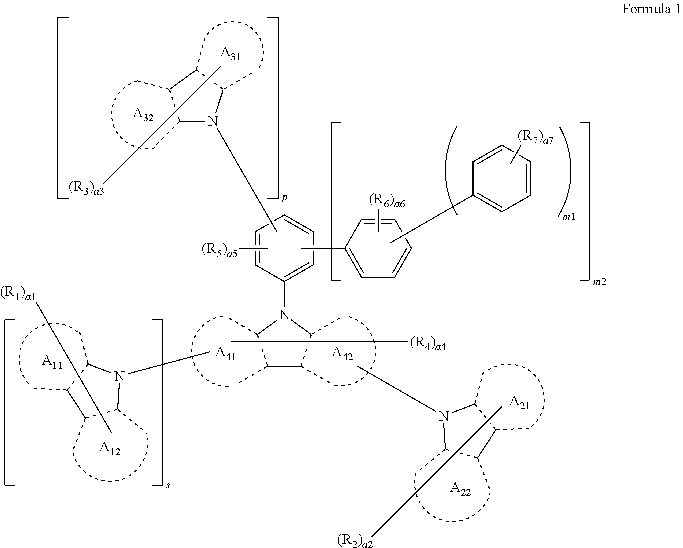
wherein, in Formula 1,
rings A₁₁, A₁₂, A₂₁, A₂₂, A₃₁, A₃₂, A₄₁, and A₄₂ are each a π electron-rich C₅-C₆₀ cyclic group,
R₁ to R₃ are each independently:
hydrogen, deuterium, a C₁-C₆₀ alkyl group, or a deuterated C₁-C₆₀ alkyl group;
a π electron-rich C₅-C₆₀ cyclic group unsubstituted or substituted with deuterium, a C₁-C₆₀ alkyl group, a π electron-rich C₅-C₆₀ cyclic group, or a combination thereof; or
*—Si(B′₁)(B′₂)(B′₃),
wherein B′₁, B′₂, and B′₃ are each a π electron-rich C₅-C₆₀ cyclic group unsubstituted or substituted with at least one W′₁,
R₄ to R₇ and W′₁ are each independently:
hydrogen, deuterium, a C₁-C₆₀ alkyl group, or a deuterated C₁-C₆₀ alkyl group; or
a phenyl group or a biphenyl group, each unsubstituted or substituted with deuterium, a C₁-C₆₀ alkyl group, a phenyl group, a biphenyl group, or a combination thereof,
wherein a1 to a4 are each independently an integer from 0 to 20,
a5 is an integer from 0 to 4,
a6 and a7 are each independently an integer from 0 to 5,
p and s are each independently 0 or 1, wherein p+s is 1 or 2,
m1 is 0, 1, 2, or 3, and
m2 is 1 or 2.

2. The composition of claim 1, wherein R₁ to R₃ in Formula 1 are each independently:
hydrogen, deuterium, a C₁-C₂₀ alkyl group, or a deuterated C₁-C₂₀ alkyl group; or
a phenyl group or an N-carbazolyl group, each unsubstituted or substituted with deuterium, a C₁-C₂₀ alkyl group, a phenyl group, an N-carbazolyl group, or a combination thereof.

3. The composition of claim 1, wherein
p in Formula 1 is 0, and
a group represented by 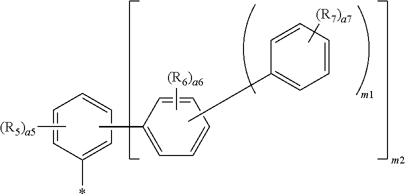
in Formula 1 is:
a group represented by one of Formulae ph-1 to ph-18; or
a group represented by one of Formulae ph-1 to ph-18, in which at least one hydrogen is substituted with deuterium: 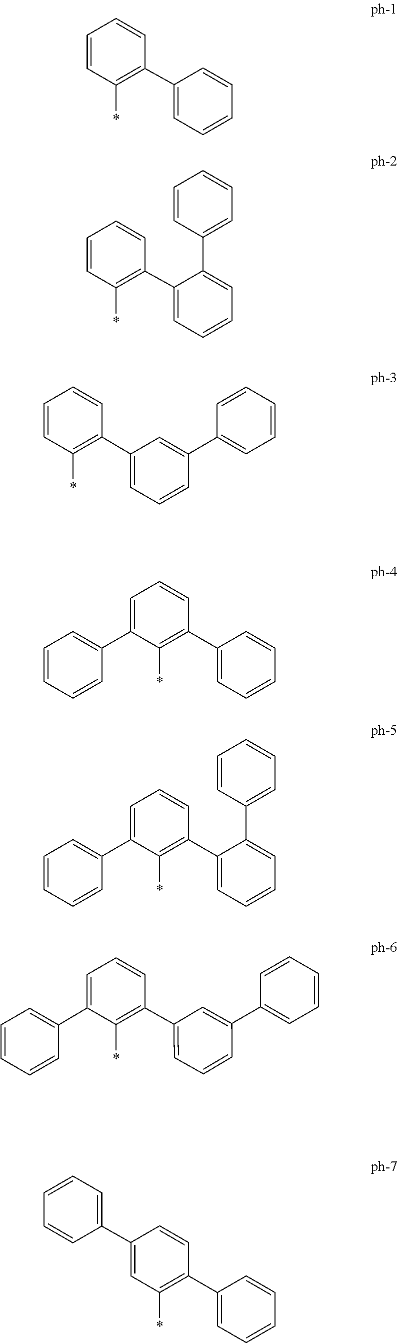 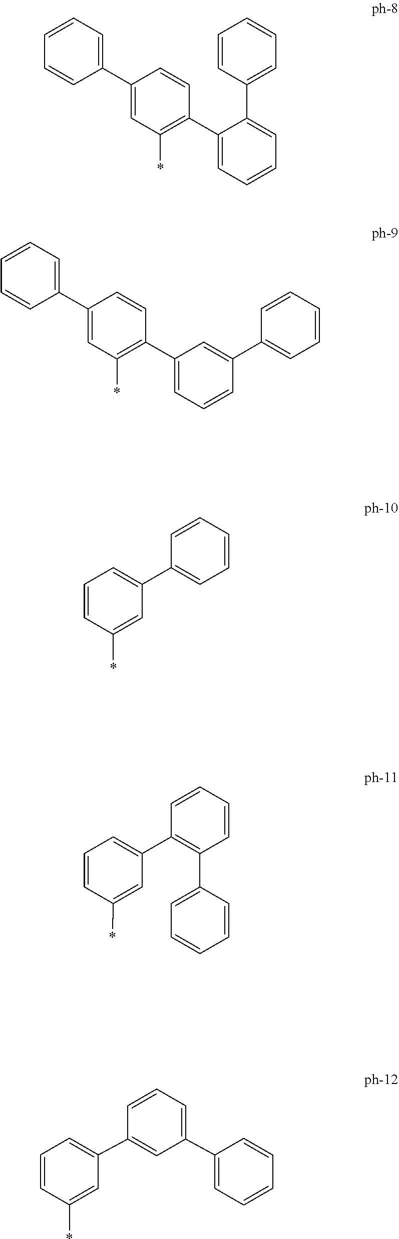 [SMILES: *c1cc(-c2ccccc2)ccc1-c1ccccc1-c1ccccc1 ; *c1cc(-c2ccccc2)ccc1-c1cccc(-c2ccccc2)c1 ; *c1cccc(-c2ccccc2)c1 ; *c1cccc(-c2ccccc2-c2ccccc2)c1 ; *c1cccc(-c2cccc(-c3ccccc3)c2)c1] 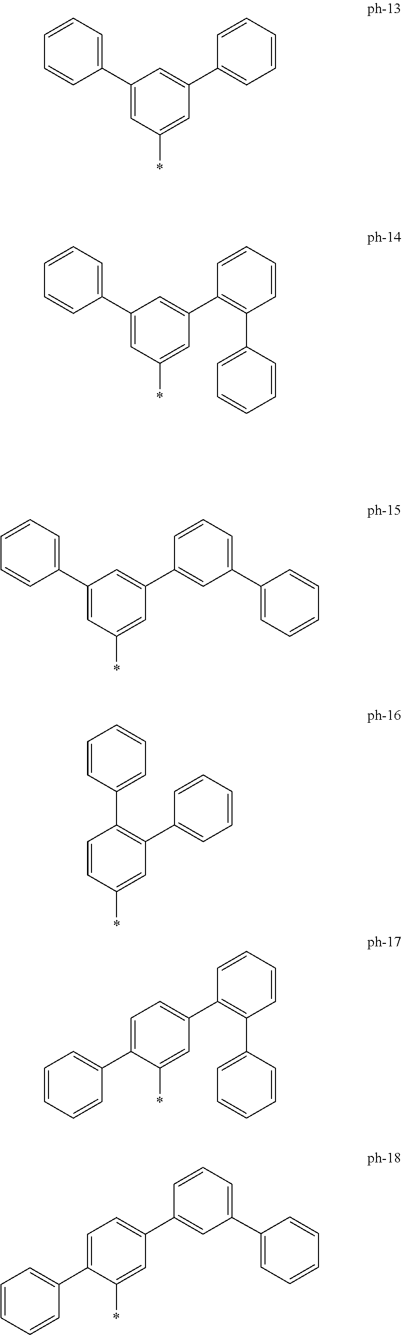 [SMILES: *c1cc(-c2ccccc2)cc(-c2ccccc2)c1 ; *c1cc(-c2ccccc2)cc(-c2ccccc2-c2ccccc2)c1 ; *c1cc(-c2ccccc2)cc(-c2cccc(-c3ccccc3)c2)c1 ; *c1ccc(-c2ccccc2)c(-c2ccccc2)c1 ; *c1cc(-c2ccccc2-c2ccccc2)ccc1-c1ccccc1 ; *c1cc(-c2cccc(-c3ccccc3)c2)ccc1-c1ccccc1]
wherein * in Formulae ph-1 to ph-18 indicates a binding site to a neighboring atom.

4. The composition of claim 1, wherein
p in Formula 1 is 1,
a group represented by 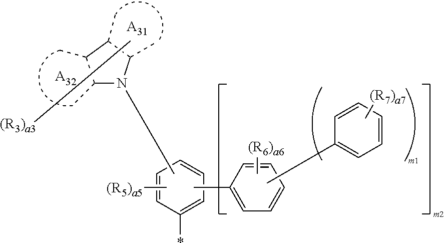
in Formula 1 is:
a group represented by one of Formulae ph-51 to ph-58; or
a group one of Formulae ph-51 to ph-58, in which at least one hydrogen is substituted with deuterium: 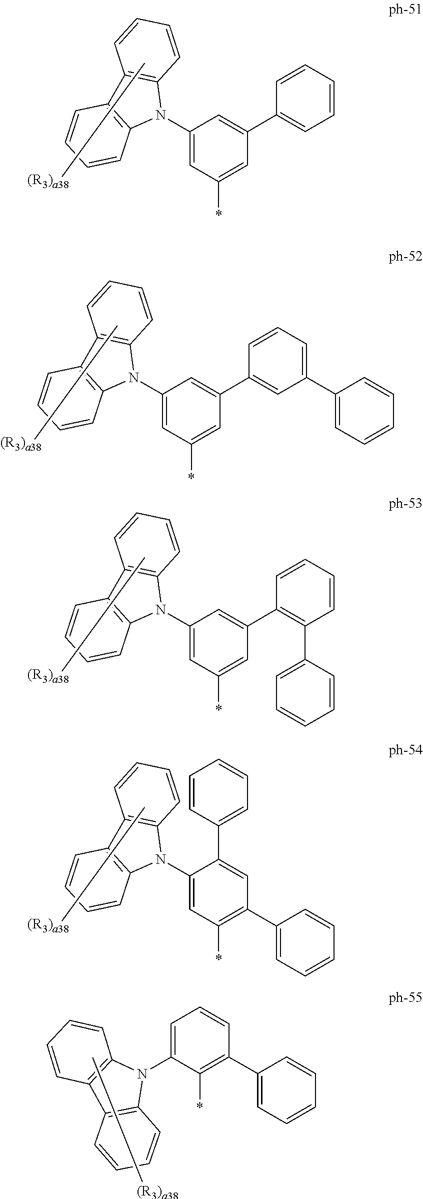 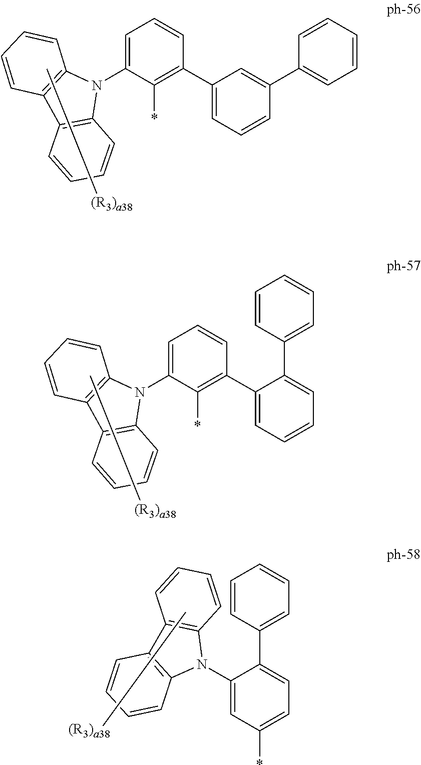
wherein, in Formulae ph-51 to ph-58,
R₃ is as defined in claim 1,
a38 is an integer from 0 to 8, and
* indicates a binding site to a neighboring atom.

5. The composition of claim 1, wherein the first compound satisfies at least one of Conditions 1 to 4:
Condition 1
the number of carbazole groups in the first compound is 3 or more
Condition 2
the number of benzene groups linked to each other only via a carbon-carbon single bond in the first compound is 2 or more
Condition 3
the first compound comprises at least one deuterium
Condition 4
the first compound comprises at least one tert-butyl group.

6. The composition of claim 1, wherein the second compound comprises:
a triazine group; and
at least one carbazole group, at least one silicon-containing group, or a combination thereof.

7. The composition of claim 1, wherein the second compound is represented by Formula 2: 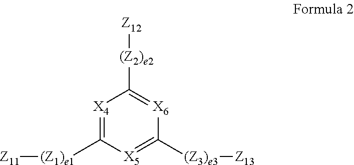
wherein, in Formula 2,
X₄ is N, C(H), or C(Z₁₄), X₅ is N, C(H), or C(Z₁₅), X₆ is N, C(H), or C(Z₁₆), wherein at least one of X₄ to X₆ is N,
Z₁ to Z₃ are each independently *—Si(Ar₁)(Ar₂)—*′, a C₅-C₃₀ carbocyclic group unsubstituted or substituted with at least one R₀, or a C₂-C₃₀ heterocyclic group unsubstituted or substituted with at least one R₀,
e1 to e3 are each independently 0, 1, 2, or 3, i) when e1 is 0, a group represented by *—(Z₁)ₑ₁—*′ is a single bond, ii) when e2 is 0, a group represented by *—(Z₂)ₑ₂-*′ is a single bond, and i) when e3 is 0, a group represented by *—(Z₃)ₑ₃-*′ is a single bond,
Z₁₁ to Z₁₃ are each independently *—Si(Ar₁₁)(Ar₁₂)(Ar₁₃), a C₅-C₃₀ carbocyclic group unsubstituted or substituted with at least one R₀, or a C₂-C₃₀ heterocyclic group unsubstituted or substituted with at least one R₀,
Ar₁, Ar₂, and Ar₁₁ to Ar₁₃ are each independently a C₅-C₃₀ carbocyclic group unsubstituted or substituted with at least one R₀ or a C₂-C₃₀ heterocyclic group unsubstituted or substituted with at least one R₀, and
R₀ and Z₁₄ to Z₁₆ are each independently:
deuterium, a C₁-C₆₀ alkyl group, or a deuterated C₁-C₆₀ alkyl group; or
a C₅-C₃₀ carbocyclic group or a C₂-C₃₀ heterocyclic group, each unsubstituted or substituted with deuterium, a C₁-C₆₀ alkyl group, a deuterated C₁-C₆₀ alkyl group, a C₅-C₃₀ carbocyclic group, a C₂-C₃₀ heterocyclic group, or a combination thereof.

8. The composition of claim 7, wherein the second compound is represented by Formula 2-1 or 2-2: 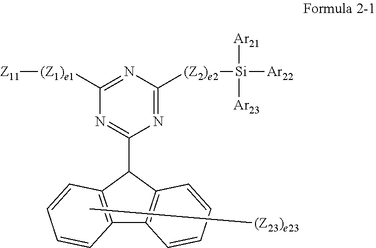 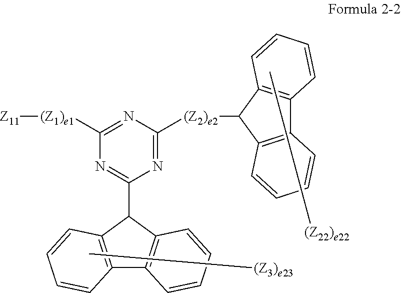
wherein, in Formulae 2-1 and 2-2,
Z₁, Z₂, e1, e2, and Z₁₁ are each as defined in claim 7,
Z₂₂ and Z₂₃ are each defined as for R₀ in claim 7,
e23 and e22 are each independently an integer from 0 to 8, and
Ar₂₁, Ar₂₂, and Ar₂₃ are defined as for Ar₁₁, Ar₁₂, and Ar₁₃ in claim 7, respectively.

9. The composition of claim 1, wherein a triplet (T₁) energy level of the first compound is about 2.8 eV or greater.

10. The composition of claim 1, further comprising an emitter.

11. The composition of claim 10, wherein the emitter emits blue light.

12. The composition of claim 10, wherein
the emitter is an organometallic compound,
the organometallic compound comprises a transition metal and n ligands linked to the transition metal, and
n is an integer from 1 to 4.

13. The composition of claim 12, wherein
the transition metal is platinum (Pt) or palladium (Pd),
n is 1, and
the ligand is a tetradentate ligand.

14. The composition of claim 13, wherein the tetradentate ligand comprises a carbene moiety linked to the transition metal.

15. The composition of claim 10, wherein
the emitter is a multiple resonance thermally activated delayed fluorescence material,
the multiple resonance thermally activated delayed fluorescence material is a polycyclic compound i) not comprising a transition metal and ii) comprising a core in which two or more C₃-C₆₀ cyclic groups are condensed to each other, and
at least two C₃-C₆₀ cyclic groups of the core are condensed to each other while sharing boron (B) or nitrogen (N).

16. The composition of claim 10, further comprising a sensitizer.

17. The composition of claim 16, wherein
the sensitizer is an organometallic compound comprising a transition metal and a tetradentate ligand linked to the transition metal,
the transition metal is platinum (Pt) or palladium (Pd), and the tetradentate ligand comprises a carbene moiety linked to the transition metal.

18. A light-emitting device comprising: a first electrode;
a second electrode; and
an interlayer between the first electrode and the second electrode and comprising an emission layer, wherein
the interlayer comprises the composition of claim 1.

19. The light-emitting device of claim 18, wherein
the composition is comprised in the emission layer, and
the emission layer emits blue light.

20. An electronic apparatus comprising the light-emitting device of claim 18.
